(19) United States
(12) Patent Application Publication
(10) Pub. No.: US 2024/0182863 A1
(43) Pub. Date: Jun. 6, 2024

(54) DECELLULARIZED LIVER FIBERS AND METHODS OF MAKING AND USING THE SAME

(71) Applicants: Colorado State University Research Foundation, Fort Collins, CO (US); University of Illinois at Chicago, Chicago, IL (US)

(72) Inventors: Matthew J. Kipper, Fort Collins, CO (US); Salman R. Khetani, Chicago, IL (US); Jennifer Liu, Chicago, IL (US); Liszt Coutinho Madruga, Fort Collins, CO (US)

(73) Assignees: Colorado State University Research Foundation, Fort Collins, CO (US); University of Illinois at Chicago, Chicago, IL (US)

(21) Appl. No.: 18/531,019

(22) Filed: Dec. 6, 2023

Related U.S. Application Data
(60) Provisional application No. 63/430,600, filed on Dec. 6, 2022.

(51) Int. Cl. C12N 5/071

(52) U.S. Cl. CPC C12N 5/067; C12N 2502/1323; C12N 2533/54; C12N 2533/90; C12N 2537/10

(57) ABSTRACT

The present disclosure relates to fibers prepared using decellularized liver ECM. The disclosure further provides in vitro cultures of human hepatocytes on decellularized liver ECM fibers, and the use of the cultures in developing and screening drugs.

### CROSS REFERENCE TO RELATED APPLICATIONS

The present application claims the benefit of U.S. Provisional Patent Application No. 63/430,600, entitled, “DECELLULARIZED LIVER NANOFIBERS ENHANCE” filed Dec. 6, 2022. The content of the aforementioned application is hereby incorporated by reference in its entirety.

### ACKNOWLEDGEMENT OF GOVERNMENT SUPPORT

This invention was made with support under Grant No. 1933552 awarded by the National Science Foundation. The government has certain rights in the invention.

### BACKGROUND

### 1. Field

The present disclosure relates to micro- or nano-fibers prepared using extracellular matrix isolated from liver, and in particular scaffolds and systems fabricated with the micro- or nano-fibers and use of the scaffolds and systems for culturing liver cells.

### 2. Background

Owing to significant differences across species in liver pathways, in vitro human liver models are utilized for screening the metabolism and toxicity of pharmaceuticals and industrial chemicals, mimicking the key aspects of liver diseases for the discovery of novel molecular targets, and building tissue surrogates for implantation into patients suffering from end-stage liver failure. Primary human hepatocytes (PHHs) are often used to fabricate such models given their ability to perform most liver functions such as protein synthesis, bile production, glucose and fatty acid metabolism, and the detoxification of endogenous and exogenous substances. However, PHHs rapidly lose phenotypic functions when cultured on their own on extracellular matrix (ECM) proteins (e.g., collagen I) adsorbed onto nonphysiological stiff substrates, such as glass and polystyrene. While the PHH phenotype can be stabilized in vitro for several weeks upon cocultivation with specific nonparenchymal cell (NPC) types, such as stromal fibroblasts, functions still remain below those in freshly isolated PHHs, partly due to the use of nonphysiologic substrates. Thus, there remains a critical need to develop more physiologic substrates for PHH and PHH/NPC cocultures alike.

The liver's ECM is composed of diverse proteins such as collagens, glycoproteins, and proteoglycans. A few attempts have been made at culturing PHHs within ECM that better mimic in vivo-like architecture and/or composition than collagen I adsorbed onto stiff substrates. For instance, sandwiching hepatocytes within two layers of gelled collagen I can induce the reformation of bile canaliculi between adjacent hepatocytes, but other functions, such as drug metabolism enzyme activities, still show a rapid decline. Similarly, culturing hepatocytes with tumor-derived murine Matrigel® can induce some functions for approximately 1 week, but extrapolating the results using Matrigel® to the effects of native liver ECM on hepatic functions is challenging. In contrast, culturing PHHs on decellularized human liver ECM can also transiently improve phenotypic functions; however, such ECM is typically variable in quality due to the unpredictable conditions of the transplant-rejected human livers. It was previously found that ECM protein composition can significantly modulate PHH functions on polyacrylamide substrates of liver-like stiffnesses in unexpected ways, but drug metabolism capacity of the cells still declined over time, partly because the substrates did not mimic the full protein composition nor the topography of liver ECM and did not provide heterotypic interactions with NPCs. Thus, more in vivo-like ECM substrates are needed to induce high and stable PHH functions for several weeks in vitro upon coculture with NPCs.

In contrast to adsorbed 2-dimensional (2D) ECM, porous 3-dimensional (3D) nanofibrous scaffolds more accurately recapitulate many features of the ECM in vivo. Electrospun nanofibrous scaffolds from natural and synthetic materials can improve the phenotype of different cell types, including primary rat hepatocytes. The large surface area of nanofiber matrices can also be used to present a high density of receptor ligands that improve hepatocyte function, including small molecules (e.g., galactose) and proteins (e.g., collagen). Others have cultured transformed human hepatocyte cell lines on electrospun nanofibers made using blends of polycaprolactone (PCL), collagen, and silk or poly (lactic-co-glycolic acid) (PLGA)-based fibers coated with collagen-I or fibronectin and shown improvements in hepatic phenotype relative to nonfibrous substrates. While the above studies demonstrate the utility of nanofibrous scaffolds for liver culture, they are either limited to synthetic fibers that do not adequately mimic the biochemistry of natural liver ECM and/or cell sources such as transformed cell lines and animal-derived hepatocytes that are known to deviate considerably from human liver functions. Thus, there remains a need to develop nanofibrous scaffolds from natural liver ECM that can support the long-term functions of PHHs.

### SUMMARY

In one aspect, the present disclosure encompasses a fiber comprising an electrospun decellularized liver extracellular matrix (ECM). In some aspects, the liver ECM is a porcine liver ECM or human liver ECM. In some aspects, the fiber is a microfiber with an average diameter of about 1000 nm. In some aspects, the fiber further comprises collagen type I. In some aspects, the fiber is a nanofiber with an average diameter of about 200 nm. In further aspects, the polymer chains within a fiber is cross-linked or the fiber is further cross-linked to another fiber.

The disclosure further encompasses a scaffold comprising the fibers described herein. A device comprising the disclosed fiber of its surface is further provided. In some aspects, the device is selected from the group consisting of a glass slide, a culture plate, a plate well, a microwell, and a microfluidic chamber device.

The disclosure further encompasses an in vitro liver cell culture comprising the fiber described herein, and liver cells. In some aspects, the liver cells are primary hepatocytes. In some aspects, the in vitro liver cell culture further comprises supportive cells, optionally wherein the supportive cells are fibroblasts. Further provided herein is a liver model system for drug or chemical screening system, the system comprising the in vitro liver cell culture described herein.

In some aspects, further provided is a method of making a liver ECM fiber. In some aspects the method can comprise a. obtaining or having obtained a decellularized porcine liver ECM; b. dissolving the decellularized liver ECM in a solvent; and c. electrospinning the solution to form a liver ECM fiber. In further aspects, the step b. further comprises dissolving collagen type I in the solvent to obtain a solution comprising a blend of decellularized liver ECM and collagen type I. In some aspects of the method, the solvent is 1,1,1,3,3,3-hexafluoro-2-propanol (HFIP). In some aspects, the decellularized liver ECM is dissolved in the solvent at about 2 (w/v) % or about 5 (w/v) %. In some aspects, the electrospinning is conducted at about 15 to about 20 KV. In some aspects, the electrospinning is conducted on the surface of a device selected from the group consisting of a glass slide, a culture plate, a plate well, a microwell, and a microfluidic chamber device. In some aspects of the method, the step a. comprises:
i. obtaining a liver tissue comprising a whole liver, a liver lobe or a portion thereof;
ii. placing the liver tissue in deionized water at about 4° C. and replacing the deionized water every hour for about 12 hours;
iii. placing the liver tissue in a sodium dodecyl sulfate (SDS) solution at about 0.1% (w/v) at about 4° C., for about 8 hours to obtain decellularized liver ECM;
iv. removing the SDS solution, and placing the decellularized liver ECM in deionized water, and replacing the deionized water every hour for about 4 hours;
v. placing the decellularized liver ECM in 75% ethanol for about 8 hours at about 4° C.;
vi. lyophilizing the decellularized liver ECM;
vii. digesting lyophilized decellularized liver ECM by dissolving in 0.1 M HCl containing 100 mg of >2500 mg/units pepsin for about 8 hours;
viii. obtaining a supernatant from the HCl solution by centrifugation at 8000 g for 10 minutes;
ix. performing dialysis on the supernatant at 7KDa MWCO; and
x. lyophilizing the supernatant to obtain a decellularized liver ECM.

In some aspects, the method can further comprise cross-linking the fiber using 1-ethyl-3-(3-dimethyl aminopropyl)carbodiimide, N-hydroxysuccinimide (NHS), or any combination thereof. In some aspects, further provided is an in vitro liver cell culture comprising the fibers produced by the method described herein, and liver cells, wherein the liver cells are primary hepatocytes. In some aspects, the in vitro liver cell culture can further comprise supportive cells, optionally wherein the supportive cells are fibroblasts.

Other technical features may be readily apparent to one skilled in the art from the following figures, descriptions, and claims.

### BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1 shows the scheme of PLECM extraction.

FIGS. 2A-2B show the preparation of in vitro liver models. FIG. 2A shows collagen extracted from rat tail and/or PLECM extracted from porcine liver were dissolved in HFIP and electrospun over 6-8 h varying the flow rate, voltage, and distance from the needle to the collector. The fibers were collected on oxidized glass. FIG. 2B shows crosslinked nanofibers were seeded with hepatocytes and/or supportive cells and cultured over several weeks.

FIGS. 3A-3B show EM images of nanofibers. FIG. 3A shows uncrosslinked nanofibers. FIG. 3B shows chemically crosslinked nanofibers.

FIGS. 4A-4B show SEM images of fibers. FIG. 4A shows uncrosslinked and FIG. 4B shows chemically crosslinked, including PLECM microfibers (left) and chitosan nanofibers (right).

FIGS. 5A-5E show physicochemical characterization of nanofibers including PLECM microfibers and chitosan nanofibers. FIG. 5A shows FTIR spectra of nanofibers, including chitosan. FIG. 5B shows FTIR of nanofibers showing specific C—N peak of PLECM. FIG. 5C shows survey XPS spectra of nanofibers. FIG. 5D shows survey XPS spectra of macromolecules. FIG. 5E shows C1s high-resolution XPS spectra of chitosan nanofibers.

FIGS. 6A-6D show physicochemical characterization of nanofibers. FIG. 6A shows C1s high-resolution XPS spectra of macromolecules. FIG. 6B shows C1s high-resolution XPS spectra of nanofibers. FIG. 6C shows FTIR of individual macromolecules. FIG. 6D shows FTIR spectra of nanofibers.

FIGS. 7A-7B show SEM and confocal microscopy images of PHH monocultures on ECM nanofibers and adsorbed collagen I control. FIG. 7A shows SEM images after 23 days of culture. FIG. 7B shows confocal microscopy images after 23 days of culture. For immunostaining, cultures were fixed and stained for antibodies against human albumin (hepatocyte marker, green), CK8 (hepatocyte marker, red), and DAPI (nucleus, blue). Scale bar for FIG. 7B is 100 μm.

FIG. 8 shows SEM images of PHH monocultures on nanofibers after 23 days including PLECM microfibers (left) and chitosan nanofibers (right).

FIGS. 9A-9B show confocal microscopy images of PHH monocultures on ECM nanofibers and adsorbed collagen control. Confocal images after 23 days of culture. Cultures were fixed and stained for antibodies against human ALB (hepatocyte marker, green, FIG. 9A), CK8 (hepatocyte marker, red, FIG. 9B) and DAPI (nucleus, blue). Scale bars=100 μm.

FIG. 10 shows confocal microscopy images of PHH monocultures on ECM nanofibers and adsorbed collagen control. Confocal images after 23 days of culture. Cultures were fixed and stained for antibodies against human ASGR1 (hepatocyte marker, red) and DAPI (nucleus, blue). Scale bar is 100 μm.

FIGS. 11A-11G show PHH monoculture viability and functions on nanofibers and adsorbed ECM controls. FIG. 11A shows Albumin secretion of PHH monocultures on the nanofibers and adsorbed ECM controls. FIG. 11B shows urea synthesis of PHH monocultures on the nanofibers and adsorbed ECM controls. FIG. 11C shows cell viability (PestoBlue) of PHH monocultures on the nanofibers and adsorbed ECM controls. FIG. 11D shows CYP1A2 enzyme activity of PHH monocultures on the nanofibers and adsorbed ECM controls. FIG. 11E shows CYP2A6 enzyme activity of PHH monocultures on the nanofibers and adsorbed ECM controls. FIG. 11F shows CYP2C9 enzyme activity of PHH monocultures on the nanofibers and adsorbed ECM controls. FIG. 11G shows CYP3A4 enzymes activity of PHH monocultures on the nanofibers and adsorbed ECM controls. Asterisks (*) indicate statistical significance relative to the adsorbed collagen control and hash (#) indicates significance relative to the adsorbed PLECM control. * or #p<0.05, ** or ##p<0.01, *** or ###p<0.001, **** or ####p<0.0001. FIG. 11A and FIG. 11B show statistical comparisons for collagen-only nanofibers, while statistical comparisons for other nanofibers are shown in Table 5.

FIGS. 12A-12G show PHH monoculture viability and functions on chitosan nanofibers, PLECM microfibers, and adsorbed ECM controls. FIG. 12A shows Albumin secretion of PHH monocultures on the nanofibers and adsorbed ECM controls. FIG. 12B shows urea synthesis of PHH monocultures on the nanofibers and adsorbed ECM controls. FIG. 12C shows cell viability (PestoBlue) of PHH monocultures on the nanofibers and adsorbed ECM controls. FIG. 12D shows CYP1A2 activity of PHH monocultures on the nanofibers and adsorbed ECM controls. FIG. 12E shows CYP2A6 activity of PHH monocultures on the nanofibers and adsorbed ECM controls. FIG. 12F shows CYP2C9 activity of PHH monocultures on the nanofibers and adsorbed ECM controls. FIG. 12G shows CYP3A4 enzymes activity of PHH monocultures on the nanofibers and adsorbed ECM controls. Asterisks (*) indicate statistical significance of PLECM microfiber relative to adsorbed collagen control and hash (#) indicates significance of PLECM microfiber relative to adsorbed PLECM control. * or #p<0.05, ** or ##p<0.01, *** or ###p<0.001, **** or ####p<0.0001.

FIGS. 13A-13B show SEM and confocal microscopy images of PHH/3T3-J2 cocultures on ECM nanofibers and adsorbed ECM control. FIG. 13A shows SEM and FIG. 13B shows confocal microscopy images after 23 days of culture. For immunostaining, the cultures were fixed and stained for antibodies against human albumin (hepatocyte marker, green), CK8 (hepatocyte marker, red), and DAPI (nucleus, blue). Scale bar for FIG. 13B is 100 μm.

FIGS. 14A-14B show confocal microscopy images of PHH/3T3-J2 co-cultures on ECM nanofibers and adsorbed ECM control. Confocal images after 23 days of culture. Cultures were fixed and stained for antibodies against human ALB (hepatocyte marker, green, FIG. 14A), CK8 (hepatocyte marker, red, FIG. 14B) and DAPI (nucleus, blue). Scale bars are 100 μm.

FIG. 15 shows confocal microscopy images of PHH/3T3-J2 co-cultures on ECM nanofibers and adsorbed ECM control. Confocal microscopy images after 23 days of culture. Cultures were fixed and stained for antibodies against human ASGR1 (hepatocyte marker, red, left column), MRP2 (hepatocyte marker, pink, middle column), ALB (hepatocyte marker, green, right column), α-SMA (3T3-J2 fibroblast marker, pink, right column), and DAPI (nucleus, blue). Scale bar is 100 μm.

FIGS. 16A-16F show PHH/3T3-J2 coculture functions on nanofibers and adsorbed ECM controls. FIG. 16A shows albumin secretion of PHH/3T3-J2 cocultures on the nanofibers and adsorbed ECM controls. FIG. 16B shows urea synthesis of PHH/3T3-J2 cocultures on the nanofibers and adsorbed ECM controls. FIG. 16C shows CYP1A2 activity of PHH/3T3-J2 cocultures on the nanofibers and adsorbed ECM controls. FIG. 16D shows CYP2A6 activity of PHH/3T3-J2 cocultures on the nanofibers and adsorbed ECM controls. FIG. 16E shows CYP2C9 activity of PHH/3T3-J2 cocultures on the nanofibers and adsorbed ECM controls. FIG. 16F shows CYP3A4 enzyme activity of PHH/3T3-J2 cocultures on the nanofibers and adsorbed ECM controls. Asterisks (*) indicate statistical significance relative to adsorbed collagen control and hash (#) indicates significance relative to adsorbed PLECM control. * or #p<0.05, ** or ##p<0.01, *** or ###p<0.001, **** or ####p<0.0001. Panels A and B show statistical comparisons for collagen-only nanofibers, while statistical comparisons for other nanofibers are shown in Table 6.

FIGS. 17A-17B show SEM and confocal microscopy images of PHH/3T3-J2 cocultures on ECM nanofibers and adsorbed ECM control. FIG. 17A shows SEM and FIG. 17B shows confocal microscopy images after 47 days of culture. For immunostaining, cultures were fixed and stained for antibodies against human albumin (hepatocyte marker, green), αSMA (fibroblast marker, pink), and DAPI (nucleus, blue). Scale bar for FIG. 17B is 100 μm.

FIGS. 18A-18F show long-term PHH/3T3-J2 co-culture functions on select nanofibers and adsorbed ECM controls. FIG. 18A shows albumin secretion of PHH/3T3-J2 cocultures on nanofibers and adsorbed ECM controls. FIG. 18B shows urea synthesis of PHH/3T3-J2 cocultures on nanofibers and adsorbed ECM controls. FIG. 18C shows CYP1A2 activity of PHH/3T3-J2 cocultures on nanofibers and adsorbed ECM controls. FIG. 18D shows CYP2A6 activity of PHH/3T3-J2 cocultures on nanofibers and adsorbed ECM controls. FIG. 18E shows CYP3A4 activity of PHH/3T3-J2 cocultures on nanofibers and adsorbed ECM controls. FIG. 18F shows CYP2C9 enzyme activity of PHH/3T3-J2 cocultures on nanofibers and adsorbed ECM controls. Asterisks (*) indicate statistical significance relative to adsorbed collagen control. * p<0.05, ** p<0.01, *** p<0.001, **** p<0.0001.

FIGS. 19A-19B show confocal microscopy images of PHH/LSEC co-cultures on ECM nanofibers and adsorbed ECM control. Confocal microscopy images after 23 days of culture. Cultures were fixed and stained for antibodies against human CD31 (LSEC marker, pink, FIG. 19A), CK8 (hepatocyte marker, red, FIG. 19B), ALB (hepatocyte marker, green) and DAPI (nucleus, blue). Scale bars are 100 μm.

FIGS. 20A-20F show PHH/LSEC co-culture and functions on nanofibers and adsorbed ECM controls. FIG. 20A shows albumin secretion of PHH/LSEC co-cultures on the nanofibers and adsorbed ECM controls. FIG. 20B shows urea synthesis of PHH/LSEC co-cultures on the nanofibers and adsorbed ECM controls. FIG. 20C shows CYP1A2 enzyme activity of PHH/LSEC co-cultures on the nanofibers and adsorbed ECM controls. FIG. 20D shows CYP2A6 enzyme activity of PHH/LSEC co-cultures on the nanofibers and adsorbed ECM controls. FIG. 20E shows CYP2C9 enzyme activity of PHH/LSEC co-cultures on the nanofibers and adsorbed ECM controls. FIG. 20F shows CYP3A4 enzyme activity of PHH/LSEC co-cultures on the nanofibers and adsorbed ECM controls. Asterisks (*) indicate statistical significance relative to adsorbed collagen control and hash (#) indicates significance relative to adsorbed PLECM control. * or #p<0.05, ** or ##p<0.01, *** or ###p<0.001, **** or ####p<0.0001. FIG. 20B shows statistical comparisons for collagen-only nanofibers, while statistical comparisons for other nanofibers are shown in Table 7.

The drawing figures do not limit the present disclosure to the specific embodiments disclosed and described herein. The drawings are not necessarily to scale, emphasis instead being placed on clearly illustrating principles of certain embodiments of the present disclosure.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

The present disclosure is partially based on development of methods by the inventors to electrospin decellularized porcine liver ECM (PLECM) into nano- and micro-fibers (approximately 200-1000 nm) without synthetic polymer blends. The nano- and micro-fibers can be used for culture of primary human hepatocytes (PHHs) in monoculture or in coculture with nonparenchymal cells, 3T3-J2 embryonic fibroblasts or primary human liver endothelial cells. The culture of PHHs on the liver ECM fibers led to higher albumin secretion, urea synthesis, and cytochrome-P450 1A2, 2A6, 2C9, and 3A4 enzyme activities than on conventionally adsorbed ECM controls. In some aspects, PHH functions showed highest activity on a collagen and PLECM blended nanofibers for about 7 weeks in the presence of the fibroblasts. The disclosed nano- and micro-fibers enhanced and stabilized PHH functions for several weeks in vitro. The nano- and micro-fiber platform disclosed herein can be used for applications, non-limiting examples of which include as in vitro human liver models useful for screening the metabolism and toxicity of compounds, modeling diseases, and cell-based therapies.

### I. Terminology

For the purposes of promoting an understanding of the principles of the present disclosure, reference will now be made to preferred aspects and specific language will be used to describe the same. Unless defined otherwise, all technical and scientific terms used herein have the meaning commonly understood by a person skilled in the art to which this invention belongs.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 wt percent to about 5 wt percent, but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In one aspect, the subject may be a rodent, e.g. a mouse, a rat, a guinea pig, etc. In another aspect, the subject may be a livestock animal. Non-limiting examples of suitable livestock animals may include pigs, cows, horses, goats, sheep, llamas and alpacas. In still another aspect, the subject may be a companion animal. Non-limiting examples of companion animals may include pets such as dogs, cats, rabbits, and birds. In yet another aspect, the subject may be a zoological animal. As used herein, a “zoological animal” refers to an animal that may be found in a zoo. Such animals may include non-human primates, large cats, wolves, and bears. In some aspects, the subject can be a human.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195, incorporated herein by reference. During this process, the dissolved polymers are placed in a dispenser. An electrostatic field is employed to generate a positively charged jet from the dispenser to the collector. Thus, a dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Reverse polarity for establishing motions of a negatively charged jet from the dispenser to the collector is also contemplated. At the critical voltage, the charge repulsion begins to overcome the surface tension of the liquid drop. The charged jets depart from the dispenser and travel within the electrostatic field towards the collector. Moving with high velocity in the inter-electrode space, the jet stretches and the solvent therein evaporates, thus forming fibers which are collected on the collector forming the electrospun scaffold.

As used herein, the term “crosslink” refers to a bond or chain of atoms attached between and linking two different molecules (e.g., polymer chains). The term “crosslinker” refers to a molecule capable of forming a covalent linkage between compounds. Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

As used herein, the term “nanofiber” refers to fibers with diameters no more than 1000 nanometers.

As used herein, the term “nanofiber scaffold” is constructed of “nanofibers.”

As used herein, the term “synthetic polymer” refers to a polymer that is synthetically prepared and that includes non-naturally occurring monomeric units. For example, a synthetic polymer can include non-natural monomeric units such as acrylate or acrylamide units. Synthetic polymers are typically formed by traditional polymerization reactions, such as addition, condensation, or free-radical polymerizations. Synthetic polymers can also include those having natural monomeric units, such as naturally-occurring peptide, nucleotide, and saccharide monomeric units in combination with non-natural monomeric units (for example synthetic peptide, nucleotide, and saccharide derivatives). These types of synthetic polymers can be produced by standard synthetic techniques, such as by solid phase synthesis, or recombinantly, when allowed.

As used herein the term “natural polymer” refers to a polymer that is either naturally, recombinantly, or synthetically prepared and that consists of naturally occurring monomeric units in the polymeric backbone.

As used herein the term “cell culture” or “culturing of cells” refers to maintaining, transporting, isolating, culturing, propagating, passaging or differentiating of cells or tissues. Cells can be in any arrangement such as individual cells, monolayers, cell clusters or spheroids or as tissue.

As used herein, the term “extracellular matrix” or “ECM” refers to a complex network of materials produced and secreted by the cells of the tissue into the surrounding extracellular space and/or medium and which typically together with the cells of the tissue impart the tissue its mechanical and structural properties. Generally, the ECM includes fibrous elements (particularly collagen, elastin, or reticulin), cell adhesion polypeptides (for e.g., fibronectin, laminin and adhesive glycoproteins), and space-filling molecules, for e.g., glycosaminoglycans (GAG), or proteoglycans.

As used here, the term “decellularization” refers to a process, wherein cells and cellular components are removed from native organs using ionic or nonionic detergents or enzymatic treatment. Decellularization eliminates the cells and cellular components of the organs while leaving surrounding structures such as the ECM and other structural molecules intact.

As used herein, the term “decellularized ECM” means extra cellular matrix that underwent a decellularization process (i.e., a removal of all cells from the organ) and is sufficiently free of cellular components.

As used herein, “sufficiently free of cellular components” refers to being more than 90%, 91%, 92%, 93%, 94%, 95%, 96%, 97%, 98%, 99%, (e.g., 100%) devoid of the cellular components present in the natural (e.g., native) tissue. As used herein, the term “cellular components” refers to cell membrane components or intracellular components which make up the cell. Examples of cell components include cell structures (e.g., organelles) or molecules comprised in same. Examples of such include, but are not limited to, cell nuclei, nucleic acids, residual nucleic acids (e.g., fragmented nucleic acid sequences), cell membranes and/or residual cell membranes (e.g., fragmented membranes) which are present in cells of the tissue. It will be appreciated that due to the removal of all cellular components from the tissue, such a decellularized matrix cannot induce an immunological response if implanted in a subject.

As used herein, the term “scaffold” refers to a three-dimensional structure comprising a biocompatible material (for e.g., micro- or nanofiber) that provides a surface suitable for adherence and proliferation of cells. A scaffold may further provide mechanical stability and support.

As used herein, the term “co-culture” refers the growth of more than one distinct cell type in a combined culture. Co-cultures of the present disclosure can include two or more distinct cell types. In some aspects, three or more distinct cell types are included in a co-culture. Co-cultures include, but are not limited to, cultures where two or more cell types are contained in the same container. This includes configurations where one or more of the cell types are contained within a transwell or similar device that is in contact with a container housing one or more cell types.

As used herein, the term “donor” includes human and other mammalian subjects from which cells such as stem cells, primary endothelial cells, and/or primary hepatocytes may be obtained.

II. Method of Making Decellularized Liver ECM fibers

In some aspects, the present disclosure encompasses a method for making nano- or micro-fibers using decellularized liver ECM. In some aspects, the disclosure further provided method of making a decellularized liver ECM.

In certain aspects, the liver for preparing decellularized ECM can be obtained from a mammal, such as for e.g., pig, rat, monkey, chimpanzee, or human. In some aspects, the liver can be obtained from a pig. In some aspects, the liver can be obtained from a human. In such aspects, the liver may be obtained from an autologous or non-autologous human subject (for e.g., from allogeneic or xenogeneic tissue). In some aspects, the liver can be removed from the mammal (for e.g., a pig) and washed for e.g., in a sterile saline solution or phosphate buffered saline (PBS), which can be supplemented with antibiotics such as Penicillin/Streptomycin. In some aspects, the liver tissue can comprise a whole liver, a liver lobe or a portion thereof, and can be used for preparing decellularized liver ECM. In some aspects, segments of liver tissues may be cut or sliced. In some aspects, the liver tissue can be fresh or frozen.

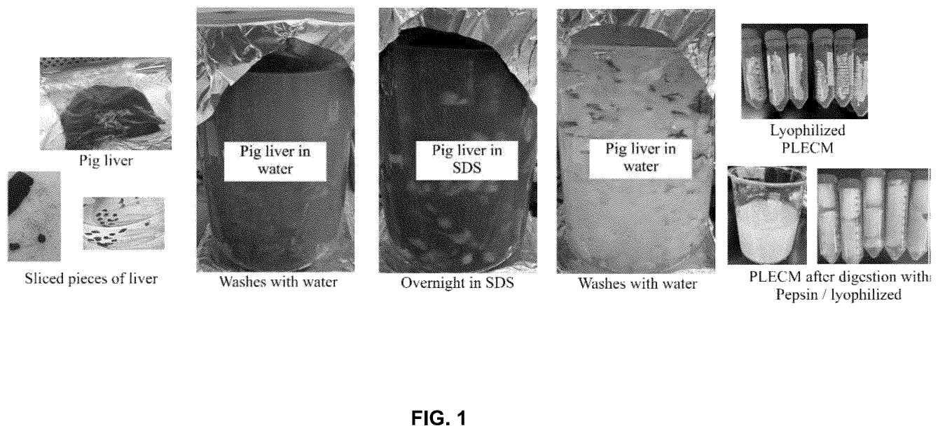

In some aspects, the liver ECM can be decellularized. Any known methods in the art can be used for decellularization of the liver ECM. In some aspects, decellularization process comprises one or more steps disclosed in FIG. 1.

In some aspects, decellularization can comprise a first step of placing the liver tissue thereof in sterile deionized water. In some aspects, the liver tissue can be further sliced, or cut into smaller pieces. In some aspects, the mixture comprising liver tissue and deionized water can further be stirred. Any known method of stirring in the art can be used, for e.g., using a magnetic stir bar, or an automatic shaker. In some aspects, the mixture can be placed at a temperature below room temperature. In some aspects, the mixture is placed in at a temperature about 1° C. to about 20° C. In some aspects, the mixture can be placed at about 4° C., about 5° C., about 6° C., about 7° C., about 8° C., about 9° C., about 10° C., about 11° C., about 12° C., about 13° C., about 14° C., or about 15° C. In some aspects, the mixture can be placed at about 4° C. In some aspects, the deionized water can be further replaced. In some aspects, the deionized water can be replaced every hour, every two hours, every three hours, every four hours, every 5 hours, or every 6 hours. In some aspects, the deionized water can be replaced every hour, or every two hours. In some aspects, the deionized water can be replaced until no blood is visible in the water.

In some aspects, the liver tissue can be placed in deionized water for about 6 hours to about 24 hours. In some aspects, liver tissue can be placed in deionized water for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more. In some aspects, the liver tissue can be placed in deionized water for about 12 hours.

In some aspects, the decellularization further comprises a second step of placing the liver tissue obtained from the previous step, in a wash solution, such as detergent solution (for e.g., ionic and nonionic detergents such as sodium dodecyl sulfate (SDS), Triton X-100, Tween-20, Tween-80). Treatment with detergent solution can help in removing the cellular components from the tissue of whole liver, a liver lobe or a portion thereof. Detergent or wash solutions can be obtained from e.g., Sigma (St Louis, Mo., USA) or Biolab (Atarot, Israel, Merck Germany).

In some aspects, the detergent solution used for decellularization according to an aspect of the present disclosure comprises SDS. In some aspects, the SDS solution can be about 0.01% to about 1% (w/v) (SDS) solution. In some aspects, the SDS solution can be about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v). In some aspects, the SDS solution can be about 0.1% (w/v) SDS solution.

In some aspects, the liver tissue can be placed in the SDS solution at a temperature about 1° C. to about 20° C. In some aspects, the liver tissue can be placed in the SDS solution can be placed at about 4° C., about 5° C., about 6° C., about 7° C., about 8° C., about 9° C., about 10° C., about 11° C., about 12° C., about 13° C., about 14° C., or about 15° C. In some aspects, the liver tissue can be placed in the SDS solution at a temperature of about 4° C.

In some aspects, the liver tissue can be placed in SDS solution for about 6 hours to about 18 hours. In some aspects, the liver tissue can be placed in SDS solution for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or more. In some aspects, the liver tissue can be placed in deionized water for about 8 hours. In some aspects, the whole liver, a liver lobe or a portion thereof can be placed in SDS solution until the tissue shows no sign of trapped blood. In some aspects, the mixture comprising the liver tissue and SDS solution can further be stirred. Any known method of stirring in the art can be used, for e.g., using a magnetic stir bar, or an automatic shaker.

In some aspects, decellularization further comprises removing the detergent solution (for e.g., SDS solution) from the decellularized liver ECM formed after treatment with the detergent solution. In some aspects, the detergent solution can be removed by subjecting the ECM to several washes in water (for e.g., deionized water) or saline (e.g., at least 10 washes of 30 minutes each, and 2-3 washes of 24 hours each), until there is no evident of detergent solution in the ECM.

In some aspects, the detergent solution can be removed from the decellularized liver ECM by washing or placing the decellularized liver ECM in deionized water. In some aspects, the deionized water can be further replaced. In some aspects, the deionized water can be replaced every hour, every two hours, every three hours, every four hours, every 5 hours, or every 6 hours. In some aspects, the deionized water can be replaced every hour. In some aspects, the deionized water can be replaced for every hour for about 2 hours, about 3 hours, about 4 hours, 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours. In some aspects, the deionized water can be replaced every hour, for about 4 hours.

In some aspects, the first step and the second step described above can be repeated. In such aspects, decellularized liver ECM can be further cut into smaller pieces, and placed in deionized water, followed by treatment with detergent solution. In some aspects, the steps can be repeated 1, 2, 3, 4, 5, 6, or more times. In some aspects, these can be repeated to achieve an decellularized liver ECM without blood seen in the decellularized liver ECM pieces.

In some aspects, optionally, the decellularized liver ECM can then be sterilized. Sterilization of the decellularized liver ECM may be effected using methods known in the art. In some aspects, the decellularized liver ECM is sterilized using ethanol. In such aspects, decellularized liver ECM can be placed in ethanol (for e.g., 70% ethanol). In some aspects, the decellularized liver ECM can be placed in ethanol for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or more. In some aspects, the decellularized ECM can be placed in ethanol for about 8 hours. In some aspects, the mixture comprising the decellularized liver ECM and ethanol can further be stirred. Any known method of stirring in the art can be used, for e.g., using a magnetic stir bar, or an automatic shaker.

In some aspects, the sterilization process can be performed at a temperature below room temperature, for e.g., about at a temperature about 1° C. to about 20° C. In some aspects, the sterilization can be performed at about 4° C., about 5° C., about 6° C., about 7° C., about 8° C., about 9° C., about 10° C., about 11° C., about 12° C., about 13° C., about 14° C., or about 15° C. In some aspects, the sterilization can be performed at a temperature of about 4° C.

Decellularized ECM can then be dried, either lyophilized (freeze-dried) or air dried. The ECM can be optionally comminuted at some point prior to enzymatic digestion, for example prior to or after decellularization and/or drying. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state. In some aspects, the decellularized liver ECM can be frozen (for e.g. in liquid nitrogen), cut into small pieces (for e.g. crumbled, crushed or ground), or lyophilized. In some aspects, the decellularized ECM can be lyophilized. Any known method in the art can be used for lyophilization, for e.g., using a benchtop lyophilizer. Lyophilization can occur at room temperature or at below room temperature, for example at 0° C., −10° C., −20° C., −30° C., and lower.

In certain aspects, the lyophilized liver ECM can be further processed. In some aspects, the lyophilized liver ECM can be treated for protein digestion. In some aspects, the lyophilized liver ECM can be treated using an acid protease. In some aspects, non-limiting examples of an acid protease can include pepsin, pepsin-like enzymes, chymosin, or rennin. In some aspects, the acid protease can be a pepsin. In some aspects, the lyophilized liver ECM can be further treated with pepsin, for e.g., in an acid solution such as a HCl solution containing pepsin.

In some aspects, digestion with pepsin can be performed at a concentration ranging from 0.01%-0.25% (w/v), more preferably, 0.02%-0.2% (w/v), more preferably, 0.05%-0.1% (w/v), even more preferably, a pepsin concentration of about 0.05% (w/v). In some aspects, pepsin used for digestion can be >2500 mg/units pepsin. In such aspects, about 1-500 mg of the >2500 mg/units pepsin can be used for digestion. In some aspects, about 1 mg, about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, or about 500 mg of >2500 mg/units pepsin can be used. In some aspects, about 100 mg of >2500 mg/units pepsin can be used.

In some aspects, HCl used for digestion can be at a concentration of about 0.01M to about 1M HCl. In some aspects, the concentration of HCl can be about 0.01M, about 0.05M, about 0.07M, about 0.1M, about 0.2M, about 0.3M, about 0.4M, about 0.5M, about 0.6M, about 0.7M, about 0.8M, about 0.9M, or about 1M. In some aspects, HCl can be used for digestion at about 0.1M. In some aspects, the lyophilized liver ECM can be treated with pepsin for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or more. In some aspects, the lyophilized liver ECM can be treated with pepsin for about 8 hours. In some aspects, the mixture comprising the HCl solution and pepsin can further be stirred. Any known method of stirring in the art can be used, for e.g., using a magnetic stir bar, or an automatic shaker.

In some aspects, the digest solution of lyophilized liver ECM can be kept at a constant stir for a certain amount of time at room temperature. The ECM digest can be used immediately or be stored at −20° C. or frozen at, for example and without limitation, −20° C. or −80° C.

In further aspects, the following the digestion, the lyophilized liver ECM can be neutralized to a pH of for e.g., 7.0-8.0, or 7.2-7.8.

In some aspects, after digestion, lyophilized liver ECM can be centrifuged, to obtain the supernatant. In some aspects, centrifugation can be performed at about 1000 g to about 10,000 g. In some aspects, centrifugation can be performed at about 100 g, about 2000 g, about 3000 g, about 4000 g, about 5000 g, about 6000 g, about 7000 g, about 8000 g, about 9000 g, or about 10,000 g. In some aspects, centrifugation can be performed at about 8000 g. In some aspects, centrifugation can be performed for about 1 minute to about 60 minutes. In some aspects, the centrifugation can be performed for about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 40 minutes, about 50 minutes, or about 60 minutes. In some aspects, the centrifugation can be performed at about 10 minutes.

In some aspects, the dialysis can be performed on supernatant thus obtained. In some aspects, dialysis can be performed at 1 kDa MWCO to 20 kDa MWCO, against an acid solution. In some aspects, the dialysis can be performed at 1 kDa MWCO, 2 kDa MWCO, 3 kDa MWCO, 4 kDa MWCO, 5 kDa MWCO, 6 kDa MWCO, 7 kDa MWCO, 8 kDa MWCO, 9 kDa MWCO, 10 kDa MWCO, 11 kDa MWCO, 12 kDa MWCO, 13 kDa MWCO, 14 kDa MWCO, 15 kDa MWCO, 16 kDa MWCO, 17 kDa MWCO, 18 kDa MWCO, 19 kDa MWCO, or 20 kDa MWCO. In some aspects, the dialysis can be performed at 7 kDa MWCO.

In some aspects, the dialysis can be performed against an acid solution. In some aspects, the acid solution non-limiting examples of which can include hydrochloric acid (HCl), acetic acid, peracetic acid, formic acid, lactic acid, citric acid, sulfuric acid, ethanoic acid, carbonic acid, nitric acid, or phosphoric acid. In some aspects, acid used for dialysis can be at a concentration of about 0.001M to about 1M HCl. In some aspects, the concentration of HCl can be about 0.001M, about 0.005M, about 0.01M, about 0.05M, about 0.07M, about 0.1M, about 0.2M, about 0.3M, about 0.4M, about 0.5M, about 0.6M, about 0.7M, about 0.8M, about 0.9M, or about 1M. In some aspects, the acid solution for dialysis can comprise HCl. In some aspects, the acid solution for dialysis comprises 0.01M HCl.

In some aspects, dialysis against an acid solution can be performed from about 1-5 days. In some aspects, dialysis can be performed from about 1 day, about 2 days, about 3 days, about 4 days, or about 5 days. In some aspects, dialysis against HCl solution can be performed for about 3 days.

In further aspects, following dialysis against an acid solution, dialysis against water can be performed. In some aspects, dialysis against water can be performed from about 1-5 days. In some aspects, dialysis can be performed from about 1 day, about 2 days, about 3 days, about 4 days, or about 5 days. In some aspects, dialysis against water can be performed for about 3 days.

In some aspects, the dialyzed ECM suspension can be powdered. In some aspects, the dialyzed ECM suspension can be processed into a powdered form by methods, for example and without limitation, such as grinding or milling in a frozen or freeze-dried state. In some aspects, the decellularized liver ECM can be frozen (for e.g. in liquid nitrogen), or lyophilized. freeze-dried.

In some aspects, the lyophilized decellularized liver ECM can be solubilized in an organic solvent. Non-limiting examples of organic solvents contemplated for use include, but are not limited to acetone, N,N-dimethylformamide (DMF), diethylformamide, chloroform, methylethylketone, acetic acid, formic acid, ethanol, 1,1,1,3,3,3-hexa fluoro-2-propanol (HFIP), tetrafluoroethanol, dichloromethane (DCM), tetrahydrofuran (THF), trifluoroacetic acid (TFA), camphorsulfonic acid, dimethyl acetamide, isopropyl alcohol (IPA), dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, acetic acid, dimethylacetamide, chloroform, dichloromethane, water, alcohols, ionic compounds, or combinations thereof. and mixtures thereof. In some aspects, the solvent can be 1,1,1,3,3,3-hexafluoro-2-propanol (HFIP). In some aspects, the lyophilized decellularized liver ECM can be dissolved in HFIP.

In some aspects, the decellularized liver ECM can be present in the organic solvent at between about 0.1 (w/v) % to about 10 (w/v) %. In some aspects, the decellularized liver ECM can be present at about 0.1 (w/v) %, about 0.5 (w/v) %, about 0.7 (w/v) %, about 1 (w/v) %, about 1.5 (w/v) %, about 2 (w/v) %, about 2.5 (w/v) %, about 3 (w/v) %, about 3.5 (w/v) %, about 4 (w/v) %, about 4.5 (w/v) %, about 5 (w/v) %, about 5.5 (w/v) %, about 6 (w/v) %, about 6.5 (w/v) %, about 7 (w/v) %, about 7.5 (w/v) %, about 8 (w/v) %, about 8.5 (w/v) %, about 9 (w/v) %, about 9.5 (w/v) %, or about 10 (w/v) %. In some aspects, the decellularized liver ECM can be present in the organic solvent at about 2 (w/v) %. In some aspects, the decellularized liver ECM can be present in the organic solvent at about 5 (w/v) %. In some aspects, the decellularized liver ECM is dissolved in HIFP at a concentration at about 2 (w/v) %. In some aspects, the decellularized liver ECM is dissolved in HIFP at a concentration of about 5 (w/v) %.

In some aspects, collagen can be further added to the solution comprising the dissolved decellularized liver ECM. In some aspects, the collagen can be collagen type I, collagen type Ill, or any combination thereof. In some aspects, the collagen can be collagen type I. In some aspects, the collagen can be dissolved in the solvent to obtain a solution comprising a blend of decellularized liver ECM and collagen. In some aspects, the collagen type I can be dissolved in the solvent to obtain a solution comprising a blend of decellularized liver ECM and collagen type 1.

In some aspects, the collagen can be dissolved in the solvent at between about 0.1 (w/v) % to about 10 (w/v) %. In some aspects, the decellularized liver ECM can be present at about 0.1 (w/v) %, about 0.5 (w/v) %, about 0.7 (w/v) %, about 1 (w/v) %, about 1.5 (w/v) %, about 2 (w/v) %, about 2.5 (w/v) %, about 3 (w/v) %, about 3.5 (w/v) %, about 4 (w/v) %, about 4.5 (w/v) %, about 5 (w/v) %, about 5.5 (w/v) %, about 6 (w/v) %, about 6.5 (w/v) %, about 7 (w/v) %, about 7.5 (w/v) %, about 8 (w/v) %, about 8.5 (w/v) %, about 9 (w/v) %, about 9.5 (w/v) %, or about 10 (w/v) %. In some aspects, the collagen can be present in the organic solvent at about 5 (w/v) %. In some aspects, the collagen type I can be present in the organic solvent at about 5 (w/v) %.

In some aspects, following the dissolution of the decellularized liver ECM, the solution can then be electrospun to form a fiber. Several parameters may affect the diameter of the fiber, including, the size of the dispensing hole of the dispenser (for e.g., a needle), the dispensing rate, the strength of the electrostatic field, the distance between the dispenser and a collector, or a solid surface (for e.g., a glass slide), and/or the concentration of the ECM used for fabricating the electro spun fiber.

In some aspects, the solution for electrospinning comprises decellularized liver ECM dissolved in HFIP at about 2 (w/v) %. In some aspects, the solution for electrospinning comprises decellularized liver ECM dissolved in HFIP at about 5 (w/v) %. In some aspects, the solution for electrospinning comprises a blend of decellularized liver ECM dissolved in HFIP at about 2 (w/v) % and collagen at about 5 (w/v) %. In some aspects, the solution for electrospinning comprises a blend of decellularized liver ECM dissolved in HFIP at about 2 (w/v) % and collagen type I at about 5 (w/v) %.

In one aspect, the fibers are electrospun with a voltage of about 0.01-20 kV, for example between about 5-30 kV, or between about 15-20 kV. In some aspects, the voltage for electrospinning the fiber can be about 0.01 kV, about 1 kV, about 5 kV, about 10 kV, about 12 kV, about 15 kV, about 16 kV, about 17 kV, about 18 kV, about 19 kV, about 20 kV, about 25 kV, or about 30 kV. In some aspects, the voltage used for electrospinning the fiber can be about 15 kV. In some aspects, the voltage used for electrospinning the fiber can be about 17 kV. In some aspects, the voltage used for electrospinning the fiber can be about 20 kV.

In some aspects, the voltage used for electrospinning a fiber comprising decellularized liver ECM is about 20 kV. In some aspects, the voltage used for electrospinning a solution comprising decellularized liver ECM dissolved in HFIP at about 2 (w/v) %, can be about 20 kV. In some aspects, the voltage used for electrospinning a solution comprising decellularized liver ECM dissolved in HFIP at about 5 (w/v) %, can be about 20 kV. In some aspects, the voltage used for electrospinning a fiber comprising decellularized liver ECM and collagen, can be about 15 kV. In some aspects, the voltage used for electrospinning a solution comprising a blend of decellularized liver ECM dissolved in HFIP at about 2 (w/v) % and collagen type I at about 5 (w/v) %, can be about 15 kV.

In some aspects, the hole of the dispenser (for e.g., a blunt needle) can be between about 1-40 gauge. In some aspects, the hole of the dispenser can be about 1 gauge, about 5 gauge, about 10 gauge, about 11 gauge, about 12 gauge, about 13 gauge, about 14 gauge, about 15 gauge, about 16 gauge, about 17 gauge, about 18 gauge, about 19 gauge, about 20 gauge, about 21 gauge, about 22 gauge, about 23 gauge, about 24 gauge, about 25 gauge, about 26 gauge, about 27 gauge, about 28 gauge, about 29 gauge, or about 30 gauge. In some aspects, the dispenser is a needle and the hole of the needle can be about 19 gauge.

In some aspects, the distance from needle to a collector, or a solid surface (for e.g., a glass slide) can be between about 1-25 cm, more preferably between 10-20 cm. In some aspects, the distance from needle to a collector can be about 1 cm, about 5 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, or about 25 cm. In some aspects, the distance from needle to a collector, or a solid surface can be about 15 cm.

In some aspects, a flow rate of electrospinning can be between about 0.01-10 ml/hr. In some aspects, the flow rate can be about 0.01 mL/h, about 0.02 mL/h, about 0.03 mL/h, about 0.04 mL/h, about 0.05 mL/h, about 0.06 mL/h, about 0.07 mL/h, about 0.08 mL/h, about 0.09 mL/h, about 0.1 mL/h, about 0.2 mL/h, about 0.3 mL/h, about 0.4 mL/h, about 0.5 mL/h, about 0.6 mL/h, about 0.7 mL/h, about 0.8 mL/h, about 0.9 mL/h, about 1 mL/h, 2 mL/h, about 3 mL/h, about 4 mL/h, about 5 mL/h, about 6 mL/h, about 7 mL/h, about 8 mL/h, about 9 mL/h, or about 10 mL/h. In some aspects, the flow rate of electrospinning can be between about 0.5 mL/h. In some aspects, the flow rate of electrospinning can be between about 1 mL/h.

In some aspects, the electrospun fibers can be collected in a collector, or on a solid surface. In some aspects, the electrospun fibers can be collected on the surface of a device. In some aspects, the device is selected from the group consisting of a glass slide, a culture plate, a plate well, a microwell, and a microfluidic chamber device. In some aspects, the collector, solid surface, or the surface of the device can be oxidized. Any known method of oxidation can be used, for e.g., via oxygen plasma.

In some aspects, the fiber can be cross-linked. In some aspects, the fiber can be crosslinked using a cross-linking agent, non-limiting examples of which can include diisocyanate compounds (such as hexamethylene diisocyanate), water-soluble carbodiimide (including 1-ethyl-3-(3-dimethyl aminopropyl)carbodiimide, or EDC), N-hydroxysuccinimide (NHS), a combination of EDC and NHS (also called EN-crosslinking), 1,4-butanediol diglycidyl ether (BD), acyl azide and dimethyl suberimidate (DMS). In some aspects, the crosslinking of the fiber is performed using a mixture of EDC (for e.g., 20 mM) and NHS (for e.g., 10 mM). In some aspects the fiber can be treated with a cross-linking agent for about 1 hour to about 36 hours. In some aspects, the fiber can be treated with a crosslinking agent for about 1 hour, 2 hours, 3 hours, about 4 hours, about 5 hours, about 10 hours, about 15 hours, about 24 hours, about 30 hours, or about 36 hours. In some aspects, the fiber can be treated with the crosslinking agent for about 24 hours.

In some aspects, cross-linking can comprise cross-linking between one or more polymer chains within a fiber. In further aspects, cross-liking can comprise cross-linking between a fiber with another fiber. In some aspect, cross-linking can comprise cross-linking fibers of a layer with nanofibers of another layer.

In some aspects, fibers adhered to the collector, solid surface, or surface of the device can be stored until further use. In some aspects, the fibers adhered to the collector, solid surface, or surface of the device can be stored at 4° C. until further use.

### III. Decellularized Liver ECM Fibers

The present disclosure further encompasses a fiber comprising an electrospun decellularized liver extracellular matrix (ECM). In some aspects, the fiber is a microfiber, or a nanofiber. The fiber disclosed according to various aspects of the disclosure, can be substantially free of a synthetic polymer. In some aspect, the fiber disclosed herein can be devoid of a synthetic fibers. In some aspects, the fiber disclosed herein consists essentially of decellularized liver ECM. In some aspects, the fiber disclosed herein consists essentially of a blend of decellularized liver ECM and collagen.

In some aspects, the fiber can be a microfiber. In some aspects, the microfiber comprises decellularized liver ECM. In some aspects, the microfiber can have an average diameter between about 900 nm-2000 nm. In some aspects, the microfiber can have an average diameter of about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, or about 2000 nm. In some aspects, the microfiber can an average diameter of about 1000 nm.

In some aspects, the fiber can be a nanofiber. In some aspects, the nanofiber comprises a blend of decellularized liver ECM and collagen. In some aspects, the nanofiber comprises a blend of decellularized liver ECM and collagen type I. In some aspects, the nanofiber can have an average diameter between about 80-500 nm. In some aspects, the nanofiber can have an average diameter of about 80 nm, about 100 nm, about 150 nm, about 200 nm, about 250 nm, about 300 nm, about 350 nm, about 400 nm, about 450 nm, or about 500 nm. In some aspects, the nanofiber can an average diameter of about 200 nm.

In some aspects, the fiber can be fabricated using a decellularized liver ECM prepared using methods disclosed herein. In some aspects, the fiber can be a blend of decellularized liver ECM and a collagen. In some aspects, a blend of decellularized liver ECM and collagen type I. In some aspects, the fiber can further be cross-linked. In some aspects, the fiber disclosed herein comprise a decellularized porcine liver ECM. In some aspects, the fiber disclosed herein comprise a decellularized human liver ECM. In some aspects, the fiber can be prepared using a solution comprising decellularized liver ECM dissolved in a solvent at about 2 (w/v) % or about 5 (w/v) %. In some aspects, the fiber can be prepared using a solution comprising decellularized liver ECM dissolved in HFIP at about 2 (w/v) % or about 5 (w/v) %. In some aspects, the fiber can be prepared using a solution comprising decellularized porcine liver ECM dissolved in a solvent at about 2 (w/v) % or about 5 (w/v) %. In some aspects, the fiber can be prepared using a solution comprising decellularized porcine liver ECM dissolved in HFIP at about 2 (w/v) % or about 5 (w/v) %. In some aspects, the fiber can be prepared using a solution comprising decellularized human liver ECM dissolved in a solvent at about 2 (w/v) % or about 5 (w/v) %. In some aspects, the fiber can be prepared using a solution comprising decellularized human liver ECM dissolved in HFIP at about 2 (w/v) % or about 5 (w/v) %. In some aspects, the fiber can be prepared using a solution comprising a blend of decellularized porcine liver ECM dissolved in a solvent at about 2 (w/v) % or about 5 (w/v) % and a collagen at about 5 (w/v) %. In some aspects, the fiber can be prepared using a solution comprising a blend of decellularized porcine liver ECM dissolved in HFIP at about 2 (w/v) % or about 5 (w/v) % and a collagen at about 5 (w/v) %. In some aspects, the fiber can be prepared using a solution comprising a blend of decellularized porcine liver ECM dissolved in a solvent at about 2 (w/v) % or about 5 (w/v) % and collagen type I at about 5 (w/v) %. In some aspects, the fiber can be prepared using a solution comprising a blend of decellularized porcine liver ECM dissolved in HFIP at about 2 (w/v) % or about 5 (w/v) % and collagen type I at about 5 (w/v) %. In some aspects, the fiber can be prepared using a solution comprising a blend of decellularized human liver ECM dissolved in a solvent at about 2 (w/v) % or about 5 (w/v) % and a collagen at about 5 (w/v) %. In some aspects, the fiber can be prepared using a solution comprising a blend of decellularized human liver ECM dissolved in HFIP at about 2 (w/v) % or about 5 (w/v) % and a collagen at about 5 (w/v) %. In some aspects, the fiber can be prepared using a solution comprising a blend of decellularized human liver ECM dissolved in a solvent at about 2 (w/v) % or about 5 (w/v) % and collagen type I at about 5 (w/v) %. In some aspects, the fiber can be prepared using a solution comprising a blend of decellularized human liver ECM dissolved in HFIP at about 2 (w/v) % or about 5 (w/v) % and collagen type I at about 5 (w/v) %.

In some aspects, the electrospun fiber can form a scaffold. In such aspects, electrospun fibers are collected in a collector, or on to a surface, and cross-linked to form a scaffold. In some aspects, scaffold can comprise fibers comprising decellularized liver ECM. In some aspects, scaffold can comprise fibers comprising a blend of decellularized liver ECM and collagen. In some aspects, scaffold can comprise fibers comprising a blend of decellularized liver ECM and collagen type I.

In some aspects, the scaffold can comprise a first layer of electrospun fibers. In some aspects, the scaffold can comprise a first layer of electrospun fibers, and a second layer of electrospun fibers. In some aspects, the scaffold disclosed herein can have multiple layers of electrospun fibers. In some aspects, a scaffold can comprise any number of layers, including about 1 layer, about 2 layers, about 3 layers, about 4 layers, about 5 layers, or more layers.

In some aspects, the scaffold disclosed according to various aspects of the disclosure, can be substantially free of a synthetic polymer. In some aspect, the scaffold disclosed herein can be devoid of a synthetic fibers. In some aspects, the scaffold disclosed herein consists essentially of decellularized liver ECM. In some aspects, the scaffold disclosed herein consists essentially of a blend of decellularized liver ECM and collagen.

In some aspects, the scaffold disclosed herein can comprise a porosity of about 10% to about 80%. In some aspects, porosity can be about 30%, about 35%, about 40%, about 45%, about 50%, about 55%, about 60%, or about 65%. In some aspects, the scaffold can have a porosity of about 45%. In some aspects, the scaffold can have a porosity of about 50%. In some aspects, the scaffold can have a porosity of about 55%. Porosity of the scaffold can be determined using any know methods in the art, including scanning electron microscopy.

In some aspects, the scaffold disclosed herein can have maximum tensile stress from about 0.01 MPa to about 10 MPa. In some aspects, Young's modulus can be about 0.01 MPa, about 0.05 MPa, about 0.1 MPa, about 0.15 MPa, about 0.2 MPa, about 0.25 MPa, about 0.3 MPa, about 0.35 MPa, about 0.4 MPa, about 0.45 MPa, about 0.5 MPa, about 0.55 MPa, about 0.6 MPa, about 0.65 MPa, about 0.7 MPa, about 0.75 MPa, about 0.8 MPa, about 0.85 MPa, about 0.9 MPa, about 0.95 MPa, about 1 MPa, about 1.5 MPa, about 2 MPa, about 2.5 MPa, about 3 MPa, about 3.5 MPa, about 4 MPa, about 4.5 MPa, about 5 MPa, about 5.5 MPa, about 6 MPa, about 6.5 MPa, about 7 MPa, about 7.5 MPa, about 8 MPa, about 8.5 MPa, about 9 MPa, about 9.5 MPa, or about 10 MPa. In some aspects, the maximum tensile stress of the scaffold can be about 0.1 MPa. In some aspects, the maximum tensile stress of the scaffold can be about 1 MPa. In some aspects, the maximum tensile stress of the scaffold can be about 5 MPa. Maximum tensile strength can be determined using any know methods in the art, including using a Instron 4442.

### IV. Cell Culture

In some aspects, the disclosure further encompasses a cell culture comprising the fiber disclosed herein. In some aspects, the cell culture comprises an in vitro cell culture comprising the fiber disclosed herein, and cells. In some aspects, the cell culture can comprise a scaffold, or a device comprising fiber disclosed herein and cells. In some aspects, a method of culturing cells is further provided. The method can comprise seeding cells in the fiber, a scaffold, or a device disclosed herein, and maintaining the culture on the fiber, scaffold, or the device.

In some aspects, the cell can be human hepatocytes. The human hepatocytes can be, for example, primary human hepatocytes (PHH), human hepatocytes derived from any human pluripotent stem cells, and an immortalized human hepatocyte cell line. The human hepatocytes may be obtained from a normal human donor or a human donor suffering from a disorder of the liver. Disorders of the liver include, but are not limited to, metabolic disorders such as Type 2 diabetes, metabolic syndrome, non-alcoholic fatty liver disease (“NAFLD”), nonalcoholic steatohepatitis (“NASH”), and cardiovascular disease. Disorders of the liver may also include infectious diseases such as hepatitis B, hepatitis C, hepatitis E, dengue fever, and ebola. The present disclosure encompasses a population of hepatocytes obtained from one or more human donors. A non-limiting, example co-culture is one which includes hepatocytes obtained from one or more human donors suffering from NAFLD or NASH. Another non-limiting, example co-culture is one which includes hepatocytes obtained from one or more normal human donors. Additionally, the human hepatocytes may be derived from any pluripotent stem cells, for example human induced pluripotent stem cells (“iPSC's”), embryonic stem cells (“ESC's”), hepatic resident stem cells (oval cells), and the like. A non-limiting, example co-culture is one which includes hepatocytes derived from human induced pluripotent stem cells such as iCell® Hepatocytes (“iHep” or “iHeps”) available from Cellular Dynamics International of Madison, WI. The pluripotent stem cell may be from a normal or a diseased donor.

In some aspects, the cell culture can further comprise supportive cells. In some aspects, the supportive cells can be an endothelial cell. In some aspects, the supportive cells can be a non-parenchymal cell. In such aspects, the cell culture comprises a co-culture of hepatocytes and supportive cells. In some aspects, the cell culture comprises a co-culture of hepatocytes and endothelial cells. In some aspects, the cell culture comprises a co-culture of hepatocytes and non-parenchymal cells.

In some aspects, the cells can comprise endothelial cells. In some aspects, endothelial cells can comprise human endothelial cells. In some aspects, the human endothelial cells can be primary human endothelial cells. In some aspects, the primary human endothelial cells can be selected from primary liver sinusoidal endothelial cells (LSECs) and human umbilical vein endothelial cells (HUVECs). In some aspects, the primary human endothelial cells can be liver endothelial cells. In some aspects, the human endothelial cells can be, for example, primary human endothelial cells, human endothelial cells derived from any human pluripotent stem cells, and an immortalized human endothelial cell line. The human endothelial cells can be obtained from a normal human donor or a human donor suffering from a disorder of the liver. The human endothelial cells may be obtained from the same individual donor as the human hepatocytes. The present disclosure encompasses a population of human endothelial cells obtained from one or more human donors. Non-limiting examples of primary human endothelial cells include human liver endothelial cells, human umbilical vein endothelial cells, human aortic endothelial cells, human coronary artery endothelial cells, human pulmonary artery endothelial cells, human dermal microvascular endothelial cells, human lymphatic endothelial cells, human splenic endothelial cells, human adrenal microvascular endothelial cells, human colonic microvascular endothelial cells, human cardiac microvascular endothelial cells, human adipose microvascular endothelial cells, human esophageal microvascular endothelial cells, human intestinal microvascular endothelial cells, human brain microvascular endothelial cells, and human lung microvascular endothelial cells. In an aspect, the primary human endothelial cells are liver endothelial cells. Specifically, the liver endothelial cells are primary liver sinusoidal endothelial cells (LSECs). In another aspect, the primary human endothelial cells are human umbilical vein endothelial cells (HUVECs). Additionally, the human endothelial cells may be derived from any pluripotent stem cells, for example human induced pluripotent stem cells (“iPSC's”), embryonic stem cells (“ESC's”), hepatic resident stem cells (oval cells), and the like. The pluripotent stem cell may be from a normal or a diseased donor. In another aspect, the human endothelial cells are an immortalized human endothelial cell line. A primary endothelial cell line may be made immortalized by methods known in the art. Specifically, the immortalized human endothelial cell line is an immortalized human-liver endothelial cell line. Non-limiting examples of immortalized human-liver endothelial cell lines include TMNK, HMEC-1, ECV304, and EaHy926 endothelial cell lines.

In some aspects, non-parenchymal cells may be human or non-human. At least one of the non-parenchymal cell populations may comprise stromal cells, such as but not limited to: fibroblasts, fibroblast-derived cells, macrophages, endothelial cells, pericytes, inflammatory cells, cholangiocytes, and other types of stromal cells, and combinations thereof. Fibroblasts may be, for example, mammalian fibroblasts, such as murine embryonic fibroblasts. Non-limiting example of murine embryonic fibroblasts include 3T3-J2, NIH-3T3, Swiss-3T3, and L1-3T3 murine embryonic fibroblasts. In an aspect, the nonparenchymal cells are 3T3-J2 murine embryonic fibroblasts. A non-limiting, example co-culture is one which includes non-parenchymal cells from normal and diseased patients. Non-parenchymal cells are obtained from one or more donors suffering from a disorder of the liver. It is contemplated that other non-parenchymal cells, both liver and non-liver, and non-parenchymal cells specifically implicated in a disease can be used for co-culture disclosed herein.

In some aspects, co-cultures of the disclosure can be established as randomly distributed co-cultures of human hepatocytes, and one of non-parenchymal cells, or human endothelial cells. In some aspects, a co-culture of human hepatocytes, and one of non-parenchymal cells, or human endothelial cells can be established by seeding both the cell populations at the same time. In other aspects, a culture of human hepatocytes can be established first, and then non-parenchymal cells or human endothelial cells can be added.

In some aspects, the cell culture comprises an in vitro liver cell culture comprising the fiber disclosed herein, and liver cells. In some aspects, the liver cells in the in vitro can be primary hepatocytes. In some aspects, the in vitro liver cell culture of comprises the fiber disclosed herein, liver cells and supportive cells. In such aspects, the supportive cells can be murine embryonic fibroblasts, or human endothelial cells.

In some aspects, the cell culture can comprise a scaffold comprising the fiber disclosed herein, and liver cells. In some aspects, the liver cells in the scaffold can be primary hepatocytes. In some aspects, the scaffold comprises the fiber disclosed herein, primary hepatocytes and supportive cells. In such aspects, the supportive cells can be murine embryonic fibroblasts, or human endothelial cells.

In some aspects, the cell culture can comprise a device comprising the fiber or scaffold disclosed herein adhered to the surface, and liver cells. In some aspects, the liver cells in the device can be primary hepatocytes. In some aspects, the device comprises the fiber or scaffold disclosed herein, primary hepatocytes and supportive cells. In such aspects, the supportive cells can be murine embryonic fibroblasts, or human endothelial cells. In some aspects, the device can be selected from the group consisting of a glass slide, a culture plate, a plate well, a microwell, and a microfluidic chamber device.

In some aspects, the fiber or scaffold for the culture can comprise a decellularized porcine liver ECM. In some aspects, the fiber or scaffold for the culture can comprise a decellularized human liver ECM. In some aspects, the fiber or the scaffold for culture can comprise decellularized liver ECM at about 2 (w/v) % or about 5 (w/v) %. In some aspects, the fiber or scaffold for the culture can comprise decellularized porcine liver ECM dissolved at about 2 (w/v) % or about 5 (w/v) %. In some aspects, the fiber or scaffold for the culture can comprise a blend of decellularized porcine liver ECM at about 2 (w/v) % or about 5 (w/v) % and collagen type I at about 5 (w/v) %.

In some aspects, the cultures described herein can be prepared using a culture medium starting with a base of, for example, Dulbecco's modified Eagle's medium (DMEM) or William's E base. The culture medium may be supplemented with various components known to facilitate growth of cells, specifically hepatocytes. In an aspect, the culture medium further comprises glucose, fetal bovine serum (FBS), insulin, an insulin-transferrin-selenium (ITS) mixture, glucagon, hydrocortisone, a buffer such as HEPES, dexamethasone, an antibiotic or antibiotic mixture such as penicillin-streptomycin, and/or growth factors. The culture medium may comprise high amount of glucose. Further, the culture medium may comprise about 5% to about 10% FBS. Additionally, the culture medium may comprise about 0.5 U/ml insulin. The culture medium may further comprise about 1% ITS. Still further, the culture medium may comprise about 5 ng/ml to about 10 ng/ml glucagon. The culture medium may also comprise about 1% to about 5% of a buffer such as HEPES. In some aspects, the culture medium may comprise bovine calf serum. In some aspects, the culture medium may comprise about 5-15% bovine calf serum. In some aspects, the culture medium comprises about 10% of bovine calf serum. Additionally, the culture medium may comprise about 100 nm dexamethasone. Also, the culture medium may comprise about 7.5 μg/ml hydrocortisone. Furthermore, the culture medium may comprise about 1% of an antibiotic or antibiotic mixture such as penicillin-streptomycin. The culture medium may also comprise a growth factor such as VEGF and FGF2. In an aspect, the culture medium comprises VEGF.

In some aspects, the culture medium can be a seeding medium comprising high glucose DMEM with 10% fetal bovine serum, 1.5% v/v HEPES buffer, 1% v/v ITS mixture, 1% penicillin/streptomycin, 100 nm dexamethasone, and 7 ng/mL glucagon. In some aspects, the culture medium can be a maintenance medium comprising high glucose DMEM with 10% fetal bovine serum, 1.5% v/v HEPES buffer, 1% v/v ITS mixture, 1% penicillin/streptomycin, 100 nm dexamethasone, 7 ng/mL glucagon, and 10% bovine calf serum. In some aspects, maintenance medium can further comprise about 20 ng/mL of VEGF protein.

In some aspects, the method of culturing can comprise obtaining the disclosed fibers, scaffold, or device, incubating the disclosed fibers, scaffold, or device in a medium. In some aspects, the fibers, scaffold, or device can be incubated in the culture medium for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or more. In some aspects, the disclosed fibers, scaffold, or device can be incubated in the culture medium for about 8 hours.

In some aspects, following incubation with a medium, the fibers or scaffold can be placed in a device, for e.g., well of a multi-well plate, transwell insert, or a microfluidic device. In some aspects, the device can be optionally pre-coated with a surfactant, for e.g., Pluronic F-127, to prevent cell attachment to surface of the substrate or the device.

In some aspects, the decellularized ECM fibers or a scaffold comprising the decellularized ECM fibers can be incorporated in a transwell insert, a culture plate, or a microfluidic device. In such aspects, the decellularized ECM fibers or a scaffold comprising the decellularized ECM fibers can be placed in the well of the insert or plate, on a culture plate, or in a chamber of the microfluidic device.

In some aspects, method of culturing can further comprise seeding of hepatocytes on to the decellularized ECM fibers or a scaffold in the device. In some aspects, the device an further comprise a culture medium. In some aspects, seeding can comprise adding about 1×10⁴ cells/ml to about 4×10¹⁰ cells/ml to the decellularized ECM fibers or a scaffold in the device. In some aspects, seeding can comprise adding about 1×10⁴ cells/ml, about 1×10⁵ cells/ml, about 1×10⁶ cells/ml, about 1×10⁷ cells/ml, about 1×10⁸ cells/ml, about 1×10⁹ cells/ml, about 1×10¹⁰ cells/ml, about 2×10⁴ cells/ml, about 2×10⁵ cells/ml, about 2×10⁶ cells/ml, about 2×10⁷ cells/ml, about 2×10⁸ cells/ml, about 2×10⁹ cells/ml, about 2×10¹⁰ cells/ml, about 3×10⁴ cells/ml, about 3×10⁵ cells/ml, about 3×10⁶ cells/ml, about 3×10⁷ cells/ml, about 3×10⁸ cells/ml, about 3×10⁹ cells/ml, about 3×10¹⁰ cells/ml, about 4×10⁴ cells/ml, about 4×10⁵ cells/ml, about 4×10⁶ cells/ml, about 4×10⁷ cells/ml, about 4×10⁸ cells/ml, about 4×10⁹ cells/ml, or about 4×10¹⁰ cells/ml. In some aspects, seeding can comprise adding about 4×10⁵ cells/ml to the to the decellularized ECM fibers or a scaffold in the device device.

In some aspects, the hepatocytes can be seeded and allowed to attach. The hepatocytes can be allowed to attach for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, 25 hours, about 26 hours, about 27 hours, about 28 hours, about 29 hours, about 30 hours, or more. In an aspect, the hepatocytes are allowed to attach for about 24 hours.

In further aspects, the device can be washed with a culture medium to remove unattached cells. In some aspects, the substrate or the device can be washed with a culture medium to remove unattached cells, about 1-3 days after seeding. In some aspects, the substrate or the device can be washed with a culture medium to remove unattached cells about 1 day after seeding.

In further aspects, the culture medium can be replaced with a maintenance medium. In some aspects, the culture medium can be replaced with a maintenance medium about 1-3 days after seeding. In some aspects, the culture medium can be replaced with a maintenance medium about 1 day after seeding. In further aspects, the maintenance medium can be replaced every about 1-2 days. In some aspects, the maintenance medium can be replaced every two days.

In further aspects, supportive cells for e.g., murine embryonic fibroblasts, or human endothelial cells can be further seeded into the decellularized ECM fibers or a scaffold in the device. In some aspects, the supportive cells can be seeded about 1 or more hours after the hepatocyte cell culture is established, about 2 or more hours after the culture is established, about 3 or more hours after the culture is established, about 4 or more hours after the culture is established, about 5 or more hours after the culture is established, about 6 or more hours after the culture is established, about 7 or more hours after the culture is established, about 8 or more hours after the culture is established, about nine or more hours after the culture is established, about 10 or more hours after the culture is established, about 11 or more hours after the culture is established, about 12 or more hours after the culture is established, about 13 or more hours after the culture is established, about 14 or more hours after the culture is established, about 15 or more hours after the culture is established, about 16 or more hours after the culture is established, about 17 or more hours after the culture is established, about 18 or more hours after the culture is established, about 19 or more hours after the culture is established, about 20 or more hours after the culture is established, about 21 or more hours after the culture is established, about 22 or more hours after the culture is established, about 23 or more hours after the culture is established, or about 24 or more hours after the culture is established. In some aspects, the supportive cells may be seeded about one or more days after the culture is established, about two or more days after the culture is established, about three or more days after the culture is established, or about four or more days after the culture is established.

In some aspects, the hepatocyte:supportive cells can be seeded at a ratio of about 1:1 to about 5:1. In some aspects, the hepatocyte:supportive cells can be seeded at a ratio of about 10:1, about 9:1, about 8:1, about 7:1, about 6:1, about 5:1, about 4:1, about 3:1, about 2:1, or about 1:1. In other aspects, the hepatocyte:fibroblast cells, or hepatocyte:endothelial cells can be seeded at a ratio of about 2:1.

The cultures, or co-cultures described herein can be maintained in vitro for at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 36 days, at least 37 days, at least 39 days, at least 40 days at least 41 days, at least 42 days, at least 43 days, at least 44 days, at least 45 days, at least 46 days, at least 47 days, at least 48 days, at least 49 days or more. In some aspects, the cultures, or co-cultures can be maintained for at least 49 days.

In some aspects, the cultures, or co-cultures described herein maintain viability of cells by at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 36 days, at least 37 days, at least 39 days, at least 40 days at least 41 days, at least 42 days, at least 43 days, at least 44 days, at least 45 days, at least 46 days, at least 47 days, at least 48 days, at least 49 days or more.

In some aspects, the viability of the cells in culture can be tested by determining for example, various cellular functions in the hepatocytes by examining gene expression, albumin production, urea production, cytochrome P450 (CYP) metabolic activity or any inducible liver enzyme activity. In some aspects, viability can be determined, for example, by measuring trypan blue exclusion, FACS analysis, or flow cytometry (i.e. using propidium iodide). Any known methods in the art, can be used for assessment, such as for e.g., ELISA, or fluorescence measurements.

### IV. Methods of Use

### In Vitro Model Systems

The present disclosure further provides an in vitro liver model for use as a system for methods involving identifying, screening, validation, or toxicity of drugs or compounds. In such aspects, the in vitro liver model comprises the decellularized ECM fibers, or a scaffold. In some aspects, the in vitro liver model comprises the decellularized ECM fibers, or a scaffold placed in a device. In some aspects, the in vitro liver model comprises the decellularized ECM fibers, or a scaffold comprising a culture of hepatocytes, placed in a device. In some aspects, the in vitro liver model comprises the decellularized ECM fibers, or a scaffold comprising a co-culture of hepatocytes and supportive cells, placed in a device. In some aspects, the in vitro liver model comprises the decellularized ECM fibers, or a scaffold comprising a co-culture of primary human hepatocytes, and primary human liver endothelial cells, placed in a device. In some aspects, the in vitro liver model comprises the decellularized ECM fibers, or a scaffold comprising a co-culture of primary human hepatocytes, and murine embryonic fibroblasts, placed in a device. In some aspects, the device can be selected from the group consisting of a glass slide, a culture plate, a plate well, a microwell, and a microfluidic chamber device. In some aspects, cultures, or co-cultures as described herein provide a model for in vitro prediction for e.g., drug induced liver toxicity, by assessing the hepatocytes.

In some aspects, the methods disclosed herein can involve determining a baseline or control value, for example, of any indicator of liver function such as gluconeogenesis, glycolysis, glycogen storage, enzyme activity, albumin secretion, urea production, gene expression, inducible liver enzyme activity and the like, in the hepatocytes in a culture, or co-culture before administering a dosage of a candidate therapeutic agent or other test agent, and comparing this with a value or level after the exposure and noting any significant change (i.e., greater than the typical margin of experimental error in repeat measurements of the same sample, expressed as one standard deviation from the mean of such measurements) over the control. In a non-limiting example, at least one indicator of hepatic function can be a measure of albumin production, urea production, ATP production, glutathione production, enzyme activity, lipid accumulation, liver gene expression, liver protein expression, or inducible liver enzyme activity in the hepatocytes.

In some aspects, the methods can involve determining a baseline or control value, for example, of any indicator of endothelial cell function such as a measure of organelle stresses (i.e., reactive oxygen species, mitochondrial membrane potential, markers of endoplasmic reticulum stress, acetylated LDL uptake) via high content imaging, visualization of fenestrae, co-staining with endothelial markers such as factor VIII and CD31, secretion of factor VIII, and the like, in the endothelial cells in co-culture before administering a dosage of a candidate therapeutic agent or other test agent, and comparing this with a value or level after the exposure and noting any significant change (i.e., greater than the typical margin of experimental error in repeat measurements of the same sample, expressed as one standard deviation from the mean of such measurements) over the control. In a non-limiting example, at least one indicator of endothelial cell function can be a measure of organelle stresses (i.e., reactive oxygen species, mitochondrial membrane potential, markers of endoplasmic reticulum stress, acetylated LDL uptake) via high content imaging, visualization of fenestrae, co-staining with endothelial markers such as factor VIII and CD31, and secretion of factor VIII.

In some aspects, the system can be utilized in various methods for identifying and screening of potential therapeutic agents, and for drug development for diseases or disorders of the liver. Disorders of the liver include, but are not limited to, metabolic disorders such as Type 2 diabetes, metabolic syndrome, non-alcoholic fatty liver disease (“NAFLD”), and non-alcoholic steatohepatitis (“NASH”). In an aspect, the hepatic disease or disorder is an infectious disease, such as hepatitis B, hepatitis C, hepatitis E, dengue fever, and ebola.

In some aspects, for example, the system of the present disclosure can be used in vitro to screen a wide variety of compounds, such as small molecules, antibodies, peptides, polypeptides, nucleic acid-based agents and the like, to identify therapeutic agents having a therapeutic effect on liver function in any disease or disorder of the liver, and/or to assess the toxicity of any such therapeutic agent before clinical implementation. For example, following contact of a culture or a co-culture with a candidate therapeutic agent, various cellular functions in the hepatocytes can assessed by examining gene expression, albumin production, urea production, cytochrome P450 (CYP) metabolic activity or any inducible liver enzyme activity, organelle stress, cell surface markers, secreted factors, uptake and secretion of liver-specific products, and response to hepatotoxins, by detecting and/or measuring level of a protein, metabolite, reporter molecule, label, or gene expression level such as through gene fluorescence in the cell or in the culture media. In non-limiting example, at least one indicator of hepatic function can be, for example, albumin production, urea production, ATP production, glutathione production, enzyme activity, lipid accumulation, liver gene expression, or liver protein expression in the hepatocytes. In a non-limiting example, at least one indicator of endothelial cell function can be a measure of organelle stresses (i.e., reactive oxygen species, mitochondrial membrane potential, markers of endoplasmic reticulum stress, acetylated LDL uptake) via high content imaging, visualization of fenestrae using electron microscopy, co-staining with endothelial markers such as factor VIII and CD31, and/or secretion of factor VIII.

In some aspects, the cultures and/or systems of the disclosure can be further used in screening for therapeutic agents for treating a disease or disorder of the liver. In some aspects, the cultures, or co-cultures can also be used in toxicology studies to determine the hepatotoxicity of an agent identified as a potential therapeutic agent. Toxicology studies can be performed on cultures, or co-cultures comprising the hepatocytes and/or human endothelial cells from human donors suffering from a disease or disorder of the liver, as described herein, which may be contrasted with comparable studies in cells from a different source. The cultures, or co-cultures described herein can be used in vitro to test a variety of potential therapeutic compounds for hepatotoxicity. Any of the screening methods described herein above can further comprise determining the toxicity of the agent by measuring in the hepatocytes and/or human endothelial cells at least one cell signal indicative of cell toxicity.

In some aspects, toxicity results can be assessed for example by observation of any of the following: a change in albumin and/or urea production, induction of any inducible liver enzyme such as cytochrome P450 (CYP) enzymes, cellular necrosis, loss of membrane integrity, cell lysis, decrease in cell viability, apoptosis, mitochondrial membrane potential, mitochondrial DNA, ER stress, organelle stress, change in endothelial cell surface markers, secretion of cell factors, and steatosis, using any one or more of vital staining techniques, ELISA assays, RT-qPCR, immunohistochemistry, imaging, electron microscopy, and the like or by analyzing the cellular content of the culture, e.g., by total cell counts, and differential cell count, by metabolic markers such as MTT and XTT, or by hepatocyte imaging technology (HIAT).

In some aspects, the cultures or, co-cultures as described herein can be exposed to varying concentrations of a candidate therapeutic agent. The amount of the candidate therapeutic agent can be, according to knowledge available to those of skill in the art, an amount representing a proposed dose or range of proposed doses in a clinical population. The time over which the hepatocytes and/or human endothelial cells are exposed to the candidate therapeutic agent can be, according to knowledge available to those of skill in the art, a period of days, weeks, or months representing time course of exposure in a clinical population. After incubation with the agent, the culture can be examined to determine the highest tolerated dose, i.e., the concentration of the agent at which the earliest morphological and/or functional abnormalities appear or can be detected. Cytotoxicity testing can also be performed using a variety of supravital dyes to assess cell viability in the culture system, using techniques known to those skilled in the art. Once a testing range is established, varying concentrations of the agent can be examined for hepatotoxic effect.

In some aspects, the present disclosure further provides a method for determining the cellular toxicity of a candidate therapeutic agent or test compound, the method comprising contacting a culture or a co-culture as described herein with the test compound; maintaining the culture or co-culture for a time and under conditions sufficient to allow an effect of the test compound on the hepatocytes and/or human endothelial cells; and taking a test measurement and/or otherwise obtaining test data indicative of a negative impact of the test compound on hepatocytes and/or human endothelial cells, which is indicative of toxicity of the test compound. The test measurement can be any measurement which provides an indicator of hepatic cell or human endothelial cells function. In a non-limiting example, at least one indicator of hepatic function can be a measure of albumin production, urea production, ATP production, glutathione production, enzyme activity, lipid accumulation, liver gene expression, liver protein expression, or inducible liver enzyme activity in the hepatocytes. In a non-limiting example, at least one indicator of endothelial cell function can be a measure of organelle stresses (i.e., reactive oxygen species, mitochondrial membrane potential, markers of endoplasmic reticulum stress, acetylated LDL uptake) via high content imaging, visualization of fenestrae using electron microscopy, co-staining with endothelial markers such as factor VIII and CD31, and/or secretion of factor VIII. For example, the test measurement can be a measurement of at least one or any combination of albumin, urea, enzyme activity, lipid accumulation, ATP production, and gene expression. The test measurement can be a measurement of at least one inducible liver {e.g., CYP) enzyme level. Test data may include applying hepatocyte imaging technology (HIAT) to the hepatocytes and/or human endothelial cells to obtain a test image. Test data can include using other imaging technology on the co-cultures, hepatocytes, and/or human endothelial cells to obtain a test image. The test measurement and/or test image can be compared to a control measurement or control image from the hepatocytes and/or human endothelial cells before contact with the test compound, and a difference between the test measurement and control measurement, or between test image and control image is indicative of toxicity of the test compound. For example, a relative decrease in albumin and/or urea production in test measurements as compared to control, following exposure of the culture or co-culture to the test compound can be indicative of hepatotoxicity.

In some aspects, the present disclosure can also provide a method of determining the toxicity arising from a drug interaction. For example, the potential toxicity of an interaction between a first test compound and a second test compound can be examined by contacting a culture or a co-culture as described herein with the first and second test compounds; maintaining the culture or a co-culture for a time and under conditions sufficient to allow an effect of an interaction between the first and second test compounds on the hepatocytes; and taking a test measurement and/or otherwise obtaining test data as described above, which can be indicative of toxicity of the interaction of the first and second test compounds.

In some aspects, the present disclosure can also provide a method of determining whether a test compound alleviates hepatic dysfunctions caused by hepatocytes and/or human endothelial cells. For example, the effects of a test compound can be examined by contacting a culture or a co-culture as described herein with the test compound; maintaining the culture or co-culture for a time and under conditions sufficient to allow an effect of the test compound on the hepatocytes and human endothelial cells; and taking a test measurement and/or otherwise obtaining test data as described above, which can be indicative of effect of test compounds on hepatic function. In some aspects more than one test compound can be examined at one time. For example, two, three, four, five, six, 7, 8, 9, or 10 test compounds can be examined.

In some aspects, the present disclosure can also provide a method for determining the effects of chronically elevated or reduced levels of glucose, fructose and/or fatty acids on the liver and liver function. The method comprises for example contacting a culture or a co-culture as described herein with a predetermined amount of one or more metabolites such as glucose, fructose, and or fatty acids, wherein the hepatocytes or human endothelial cells are obtained from one or more human donors suffering from a disorder of the liver; maintaining the culture or co-culture for a time and under conditions sufficient for the hepatocytes to generate a signal indicative of modified cellular function induced by the predetermined amount of one or more metabolites; and detecting the signal generated by hepatocytes in the presence of the one or more metabolites, wherein the signal relative to a signal generated in a control cell subject to the same conditions can be indicative of an effect of the amount of the one or more metabolites on the hepatocytes. The signal indicative of an effect on cell function may be a change in transcription, translation or secretion of a protein, cellular necrosis, loss of membrane integrity, cell lysis, decrease in cell viability, apoptosis, mitochondrial membrane potential, mitochondrial DNA, ER stress, and steatosis. The predetermined amount can be an amount which is elevated or reduced relative to a control amount which is representative of an amount of each metabolite which is considered within the range of normal in vivo values for the metabolite. The time over which the hepatocytes are exposed to the elevated or reduced level(s) of metabolite(s) can be, according to knowledge available to those of skill in the art, a period of days, weeks or months representing chronic elevation or reduction of the metabolite in a clinical population.

It should be understood that many other signals of toxicity from the hepatocytes and/or human endothelial cells can be detected and/or measured and compared to controls to identify and/or quantify toxicity induced by a candidate therapeutic agent, wherein the signal relative to a signal generated in a control culture or co-culture can be indicative of a toxic effect of the candidate agent on the hepatocytes and/or human endothelial cells. Such signals include, in non-limiting example, cellular necrosis, loss of membrane integrity, cell lysis, decrease in cell viability, apoptosis, mitochondrial membrane potential, mitochondrial DNA, ER stress, and steatosis, any one of which can be readily measured using techniques and materials known in the art.

### Devices

In some aspects, the disclosure can further encompass devices that allow for culture or co-culture described herein. In some aspects, the device can be a microfluidic device, or a multi-well plate (for e.g., a 96-well plate or a 384-well plate). In such aspects, culture or co-culture of liver cells can be established in one or more chambers or wells of the device and can be used as a system to test or screen for therapeutic compounds or assessing hepatic function in vitro. In some aspects, the present disclosure contemplates a method of culturing cells, comprising providing a microfluidic device comprising decellularized liver ECM fibers, or scaffold described herein, seeding primary human hepatocytes, and optionally supportive cells on the decellularized liver ECM fibers, or scaffold, and culturing said seeded cells under flow conditions such that said cells remain viable for at least a period of time.

In some aspects, one or more chambers can each independently comprise a channel. The channel(s) can be substantially linear or they can be non-linear. In some aspects, the channels are not limited to straight or linear channels and can comprise curved, angled, or otherwise non-linear channels. It is to be further understood that a first portion of a channel can be straight, and a second portion of the same channel can be curved, angled, or otherwise non-linear. Without wishing to be bound by a theory, a non-linear channel can increase the ratio of culture area to device area, thereby providing a larger surface area for cells to grow. This can also allow for a higher amount or density of cells in the channel.

In some aspects, the device in accordance with an aspect of the disclosure includes one or more inlet fluid ports in communication with one or more corresponding inlet apertures located on an outer surface. In some aspects, the device can be connected to a fluid source via the inlet aperture in which fluid travels from the fluid source into the device through the inlet fluid port. In some aspects, the device can further include one or more outlet fluid ports in communication with one or more corresponding outlet apertures on the outer surface. In some aspects, a fluid passing through the device can exit the device to a fluid collector or other appropriate component via the corresponding outlet aperture.

In some aspects, the device can comprise an inlet channel connecting an inlet fluid port to a first chamber. The inlet channels and inlet ports can be used to introduce cells, agents (for e.g., but not limited to, drug candidate), air flow, and/or cell culture media into the first chamber. In some aspects, device can also comprise an outlet channel connecting an outlet fluid port to the first chamber. The outlet channels and outlet ports can be used to remove cells, agents, and/or cell culture media from the first chamber.

In some aspects, the device can comprise one or more chambers. In such aspects, the fluid passing between the inlet and outlet fluid ports can be shared between the one or more chambers. In some aspects, the device can further include one or more pressure inlet ports and one or more pressure outlet ports in which the inlet ports are in communication with corresponding apertures located on the outer surface of the device. In some aspects, the inlet and outlet apertures can be on the top surface, or be located on one or more lateral sides. In operation, one or more pressure tubes connected to an external force source (for e.g., pressure source) can provide positive or negative pressure to the device via the apertures. Additionally, pressure tubes can be connected to the device to remove the pressurized fluid from the outlet port via the apertures. In some aspects, the pressure apertures can be on the top surface of the first structure, or can be located on one or more side surfaces of the device.

In some aspects, one or more fluid tubes connected to a fluid source can be coupled to the aperture to provide fluid to the device. In some aspects, fluid can exit the device via the outlet port and outlet aperture to a fluid reservoir/collector or other component. In further aspects, the device can further comprise a removable cover.

In some aspects, the width of the one or more chambers can vary with desired cell growth surface area. In some aspects, the one or more chambers can each have a range of width dimension between 100 microns and 50 mm, or between 200 microns and 10 mm, or between 200 microns and 1500 microns, or between 400 microns and 1 mm, or between 50 microns and 2 mm, or between 100 microns and 5 mm. In some aspects, the widths of the one or more chambers can be configured to be different. In some aspects, the channel heights, widths, and/or cross sections can vary along the length of the devices described herein.

In some aspects, height of the one or more chambers can be of any dimension, for e.g., sufficient to accommodate cell height and/or to permit a low shear flow. For example, in some aspects, the height of the chamber can range from about 100 μm to about 50 mm, about 200 μm to about 10 mm, about 500 μm to about 5 mm, or about 750 μm to about 2 mm.

In some aspects, the devices described herein can be manufactured using any conventional fabrication methods, including, e.g., injection molding, embossing, etching, casting, machining, stamping, lamination, photolithography, or any combinations thereof. In some aspects, the devices described herein can be produced as a monolithic device or as individual components, which can then be assembled together to form a device described herein. Each individual component can be produced by a conventional manufacturing method such as injection molding, extrusion, casting, lamination, embossing, compression molding, solvent casting, an additive manufacturing method (e.g., 3D printing), or any combinations thereof.

““In various aspects, the device disclosed herein can be used for screening of potential therapeutic agents, and/or assessing toxicity of compounds. In some aspects, further provided are methods of using the device for screening of therapeutic agents and/or assessing toxicity of compounds. The method can comprises providing at least one device according to one or more aspects described herein, and flowing fluid through the one or more chambers comprising the culture or co-culture, detecting response of the cells (for e.g., hepatocytes) in the device, detecting at least one component (for e.g., a molecule secreted or consumed by the cells in the device) present in an output fluid from the device.

In some aspects, the device can be a multi-well plate such as for e.g., 6-well, 24-well, 96-well, or 384-well plate. In some aspects, the multi-well plate can be those having at least 384, 864, 1536 wells, or a greater number of wells. In such aspects, the decellularized liver ECM fibers or a scaffold comprising the decellularized liver ECM can be placed in one or more wells in the device. In further aspects, a culture medium can be added to the one or more wells of the device. In some aspects, each well of the plate can comprise at least about 10, 15, 20, 25, 30, 35, 40, 45, 50, 55, 60, 65, 70, 75, 80, 85, 90, 95, 100, 150, 200, 250, 300, 350, 400, 450, 500 microliters, or more of the culture medium. In some aspects, the number of the cells (for e.g., hepatocytes, and/or supportive cells) seeded in the each well of the plate can be optimized based on the dimension of the well. In some aspects, cells can be seeded at about 250, about 500, about 1,000, about 2000, or about 3,000 to any of about 5,000, about 6,000, about 7,000, about 7500, about 8000 or about 9,000 cells per well. In some aspects, any of about 5,000, about 10,000, about 15,000, or about 20,000 to any of about 15,000, about 20,000, about 25,000, or about 30,000 cells can be seeded per well. In some aspects, about 10,000, about 15,000, about 20,000, about 25,000, about 30,000, or about 35,000 to any of about 60,000, about 65,000, about 70,000, about 75,000, about 80,000, about 85,000, or about 90,000 cells can be seeded per well.

In some aspects, the devices described herein can be used to determine an effect of a test agent (e.g., toxicity) by culturing cells as described herein, in the devices. Accordingly, in some aspects, the method can further comprise contacting the cells (for e.g., hepatocytes) with a with a test agent, described herein. In some aspects, the effects of the test agent on the cells can be determined by measuring response of the cells to the test agent, the fluid exiting the one or more chambers, or the wells, or using a high throughput screening microscope, and comparing the measured response with the cells not contacted with the test agent.

In some aspects, the cells are patient-specific, exposure of the patient-specific cells to a test agent, followed by determination of the effect of the test agent on the cells can facilitate identification of a personalized treatment for a subject.

In some aspects, a liver-specific condition can be created by genetically modifying normal healthy cells, for e.g., by silencing one or more genes, or over-expressing one or more genes. Methods of gene silencing include, but are not limited to, RNA interference (e.g., but not limited to small interfering RNA (siRNA), microRNA (miRNA), and/or short hairpin RNA (shRNA)), antisense oligonucleotides, ribozymes, triplex forming oligonucleotides, and the like.

In some aspects, the devices described herein are used to create a disease-specific model, the devices can further comprise normal healthy cells (for e.g., obtained from one or more healthy donors) cultured in a separate chamber, for e.g., to create a baseline for comparison. In some aspects, the device can comprise both healthy and disease-specific cells. In some embodiments, the device can include only disease-specific cells.

Various methods to measure cell response are known in the art, including, but not limited to, cell labeling, immunostaining, optical or microscopic imaging (e.g., immunofluorescence microscopy and/or scanning electron microscopy), spectroscopy, gene expression analysis, cytokine/chemokine secretion analysis, metabolite analysis, polymerase chain reaction (PCR), immunoassays, ELISA, gene arrays, spectroscopy, immunostaining, electrochemical detection, polynucleotide detection, fluorescence anisotropy, fluorescence resonance energy transfer, electron transfer, enzyme assay, magnetism, electrical conductivity (e.g., trans-epithelial electrical resistance (TEER)), isoelectric focusing, chromatography, immunoprecipitation, immunoseparation, aptamer binding, filtration, electrophoresis, use of a CCD camera, mass spectroscopy, or any combination thereof. Detection, such as cell detection, can be carried out using light microscopy with phase contrast imaging and/or fluorescence microscopy based on the characteristic size, shape and refractile characteristics of specific cell types. Greater specificity can be obtained using optical imaging with fluorescent or cytochemical stains that are specific for individual cell types (for e.g., hepatocytes).

In some aspects, the devices described herein can be utilized in an overall system incorporating sensors, computers, displays and other computing equipment utilizing software, data components, process steps and/or data structures.

### Tissue Engineering and Regenerative Medicine

In further aspects, the culture, co-culture, or methods described herein can be used to develop personalized medicine. It has been found that certain correlations can exist between an individual subject's particular genotype with respect to specific molecular markers, and drug treatment efficacy. Any of the co-cultures and methods described herein can also be used to develop personalized medicine, to determine whether any such correlation exists between a particular genotype and selected drug treatment for a disease or disorder of the liver. For example, cultures or co-cultures can be prepared using human hepatocytes and/or human endothelial cells derived from pluripotent stem cells obtained from a variety of donors of different genotypes, and any therapeutic candidate can be tested for efficacy against each genotype to determine whether any one or subset of the tested genotypes fares better or worse with a given therapeutic candidate. Any therapeutic candidate can be tested for effect on any inducible liver enzymes, and/or for a negative interaction with a second therapeutic candidate. Such information considered together with the genotype of an individual patient, can be used by a health care provider to determine a treatment option with the highest likelihood of efficacy for the individual subject, and/or to determine a risk of a negative side effect in the individual subject from a therapeutic candidate. In another aspect, in order to fit the best drug to the individual subject, a tissue biopsy of the subject's liver can be to extract liver cells for the culture or co-culture described herein. Different therapeutic candidates or drugs can be tested to help determine the extent to which the individual's liver cells responds to the candidates or drugs.

In further aspects, bioagents can be added to preserve the decellularized ECM, or scaffold or to prepare the decellularized ECM or scaffold for recellularization and/or to assist or stimulate cells during the recellularization process. Such bioagents include, but are not limited to, one or more growth factors (e.g., VEGF, DKK-1, FGF, BMP-1, BMP-4, SDF-1, IGF, HGF, Activin A, Retinoic Acid, and bFGF,), immune modulating agents (e.g., cytokines, glucocorticoids, IL2R antagonist, leucotriene antagonists), chemical (clozapine-N-oxide, phosphoinositide-3-kinase inhibitor, and Nicotinamide) and/or factors that modify the coagulation cascade (e.g., aspirin, heparin-binding proteins, and heparin). In addition, a decellularized ECM or scaffold described herein can be further treated with, for example, irradiation (e.g., UV, gamma) to reduce or eliminate the presence of any type of microorganism remaining on or in a decellularized ECM.

In further aspects, the culture, or co-culture described herein can be used for tissue engineering and/or wound healing. In such aspects, the culture, or co-cultures prepared via the methods disclosed herein can be used for regenerative medicine, for e.g., to replace, or reconstruct failing livers. The cells for the culture can be obtained from the subject, or a donor, or group of donors. In some aspects, In those cases, the cells used for tissue engineering can be obtained from the subject such that the cells are “autologous” to the subject. In some aspects, cells from a subject can be obtained from, for example, by tissue biopsy at different stages of life (e.g., prenatally, neonatally or perinatally, during adolescence, or as an adult) using methods known in the art. Alternatively, the cells used for tissue engineering can be syngeneic (i.e., from an identical twin) to the subject. In some aspects, cells can be allogeneic to the subject from, for example, a non-HLA-matched donor.

Cells may be isolated using techniques known to those skilled in the art. For example, the tissue can be cut into pieces, disaggregated mechanically and/or treated with digestive enzymes and/or chelating agents that weaken the connections between neighboring cells making it possible to disperse the tissue into a suspension of individual cells without appreciable cell breakage. If necessary, enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, and dispase. Mechanical disruption can also be accomplished using a number of methods including, but not limited to, scraping the surface of the tissue, the use of grinders, blenders, sieves, homogenizers, pressure cells, or insonators to name but a few.

In some aspects, isolated cells may be transfected with a nucleic acid sequence. Useful nucleic acid sequences may be, for example, genetic sequences which reduce or eliminate an immune response in the host. For example, the expression of cell surface antigens such as class I and class II histocompatibility antigens may be suppressed. In addition, transfection can also be used for gene delivery. Cells may be transfected with specific genes prior to seeding onto the fiber, or scaffold described herein. In some aspects, the cultured cells can be engineered to express gene products that would produce a desired protein that helps ameliorate a particular disorder. Methods for genetically engineering cells for example using retroviral vectors, adenoviral vectors, adeno-associated viral vectors, polyethylene glycol, or other methods known to those skilled in the art can be used. These include using expression vectors which transport and express nucleic acid molecules in the cells. Vector DNA is introduced into prokaryotic or eukaryotic cells via conventional transformation or transfection techniques. Suitable methods for transforming or transfecting host cells can be found in Sambrook et al. Molecular Cloning: A Laboratory Manual, 2nd Edition, Cold Spring Harbor Laboratory press (2001), and other laboratory textbooks.

In some aspects, the fibers, the scaffold, culture, or co-culture described herein can further comprise a bioactive agent. Bioactive agents can include one or more of the following: chemotactic agents; therapeutic agents (e.g., antibiotics, steroidal and non-steroidal analgesics and anti-inflammatories (including certain amino acids such as glycine), anti-rejection agents such as immunosuppressants and or other drugs); various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGFβ I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate. In some aspects, bioactive agents can include chemokines such as, without limitation: CCL21, CCL22, CCL2, CCL3, CCL5, CCL7, CCL8, CCL13, CCL17, CXCL9, CXCL10, and CXCL11, cytokines such as without limitation IL-2 subfamily cytokines, interferon subfamily cytokines, IL-10 subfamily cytokines, IL-1, 1-18, IL-17, and tumor necrosis factor. In some aspects, the bioactive agent can be small molecule drugs/therapeutic agents including, without limitation, simvastatin, kartogenin, retinoic acid, paclitaxel, vitamins (e.g., vitamin D3), etc. In some aspects, the bioagent can be a blood clotting factor such as thrombin or fibrinogen. In addition, the growth factor can be autologous growth factor that is supplied by platelets in the blood. In this case, the growth factor from platelets will be an undefined cocktail of various growth factors. In some aspects, bioactive agents include culture media, bone morphogenic proteins, growth factors, growth differentiation factors, recombinant human growth factors, cartilage-derived morphogenic proteins, antibiotics, hydrogels, polymers, anti-inflammatory medications, immunosuppressive mediations, autologous, allogenic or xenologous cells such as stem cells, chondrocytes, fibroblast and proteins such as collagen and hyaluronic acid. Bioactive agents can be autologous, allogenic, xenogeneic or recombinant.

In some aspects, the decellularized ECM fiber can be autologous, allogeneic or xenogeneic to a subject. In certain aspects, the decellularized ECM fibers, or scaffold of the present disclosure can be cultured with cells in vivo (for e.g., after the decellularized ECM fibers or scaffold has been transplanted into a subject). In some aspects, the decellularized ECM fiber, or scaffold, with or without culture or co-culture of cells can be transplanted or implanted into a subject.

In further aspects, the progress of cellularization of the transplanted decellularized ECM fivers or the scaffold can be monitored. In some aspects, monitoring comprises determining whether or not various markers or functions are present in a cell or a population of liver cells. Markers associated with different liver cells types and different stages of differentiation for those cell types are known in the art, and can be readily detected using antibodies, metabolic profiles, standard immunoassays, metabolic capabilities, physiological responses, etc. Nucleic acid assays as well as morphological and/or histological evaluation can be used to monitor organ function.

### V. Kits

The present disclosure provides kits comprising decellularized ECM fiber, a scaffold, culture, co-culture, or a device for use with a method of the disclosure. The kits may comprise a decellularized ECM fiber, scaffold, culture, co-culture, or a device, and instructions for using. The kit could further comprise cells, for e.g., hepatocytes and/or supportive cells. In some aspects, the kits can further comprise a medium, for e.g., a culture medium. The kits provided herein generally include instructions for carrying out the methods. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The disclosed kits may have a single container that contains the disclosed decellularized ECM fiber, a scaffold, culture, co-culture, or a device with or without any additional components, or they may have distinct containers for each desired agent. Where combined, they are provided, a single solution may be pre-mixed, either in a molar equivalent combination, or with one component in excess of the other. Alternatively, each of the decellularized ECM fiber, a scaffold, culture, co-culture, or a device and other components of the kit may be maintained separately within distinct containers.

In some aspects, a kit for determining the effect of a test agent on hepatocytes is further provided. A kit may comprise for example: a population of human hepatocytes, optionally, one non-parenchymal cell population, and/or a population of human endothelial cells, for preparing a culture or co-culture as disclosed herein. In one aspect, the hepatocytes may be obtained from one or more human donors suffering a disorder of the liver. The kit may further comprise a culture medium as described herein, and/or additional materials or reagents for testing various biological activities of the cells in culture. For example, the kit may contain separately packaged amounts of a culture medium, a stain or dye such as but not limited to a fluorimetric dye, a lipid dye such as Nile red, and/or a cellular stain for glycogen such as PAS stain. The kit may further comprise one or more devices described herein.

In some aspects, the kit may further comprise a reporter molecule or label capable of generating a signal indicative of a level of a cellular activity of interest in the hepatocytes, such as but not limited a vital dye, a lipid dye, a colorimetric agent, or a bioluminescent marker. The kit may include a detectable label such as a fluorophore, a radioactive moiety, an enzyme, a chromophore, a chemiluminescent label, or the like, and/or reagents for carrying out detectable labeling. The labels and/or reporter molecules, any calibrators and/or controls can be provided in separate containers or pre-dispensed into an appropriate assay format, for example, into microtiter plates.

It is contemplated for example that one or more of the presently disclosed co-cultures can be provided in the form of a kit with one or more containers such as vials or bottles, with each container containing a separate population of cells and/or reagents and washing reagents employed in an assay. The kit can comprise at least one container for conducting the assay, and/or a buffer, such as an assay buffer or a wash buffer, either one of which can be provided as a concentrated solution, a substrate solution for the detectable or a stop solution. The kit may comprise all components, e.g., reagents, standards, buffers, diluents, etc., which are necessary to perform the assay. The kit may contain instructions for determining the presence or amount of any metabolite, biomarker, label, or reporter of interest in the co-culture, in paper form or computer-readable form, such as a disk, CD, DVD, or the like, and/or may be made available online.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (for e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

It will be readily apparent to those skilled in the art that other suitable modifications and adaptations of the methods of the disclosure described herein are obvious and may be made using suitable equivalents without departing from the scope of the disclosure or the embodiments disclosed herein. Having now described the present disclosure in detail, the same will be more clearly understood by reference to the following examples, which are included for purposes of illustration only and are not intended to be limiting of the disclosure.

### EXAMPLES

The following examples are included to demonstrate aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Methods

Collagen Extraction from Rat Tail Tendon

Type I collagen extraction from rat tail tendons followed procedures described previously in the literature, with some modifications as described below. Rat tails were kindly donated by the rocky mountain raptor program (Fort Collins, CO). The tails were kept frozen in a −80° C. freezer until the extraction.

The In a biosafety cabinet, 5-10 rat tails were thawed and disinfected in a 600 mL beaker containing 70% (v/v) ethanol solution. The skin was peeled off by fine scissors and the tails were placed in 600 mL sterile deionized water (DI water). Three sterile large petri dishes were placed inside of the biosafety cabinet, one of them filled with sterile 1% (w/v) NaCl solution and the other two with sterile DI water. The rat tails were dissected using hemostats to break the vertebra every 1-2 cm and pulled out and recover the tendons, which were placed first in the petri dish containing the NaCl solution, with the aid of sterile tweezers, for washing and removing excess blood and particles, followed by rinsing in the two other petri dishes with DI water. The rinsed tendons were placed into a 1 L beaker containing 800 mL of 3% acetic acid solution and stirred overnight at 4° C. in a cold room. On the next day, in a biosafety cabinet, the translucent collagen suspension with some particles was passively filtered through four layers of cheesecloth atop a Buchner funnel into a 1 L beaker. The filtrate was transferred into 6 sterile centrifuge bottles (250 mL) weighing not more than 1 g of difference between each other and centrifuged for 2 hours at 4° C. at 8590 rpm in a Thermo Sorvall Legend XTR Refrigerated Centrifuge. After that, in a biosafety cabinet, the collagen suspension supernatant was recovered into a sterile 4 L beaker and the small pellet was discarded. A sterile separation funnel (500 mL) was set in a ring stand, raised above the beaker, and filled with 400 mL of sterile 30% (w/v) NaCl solution. The NaCl solution was slowly dripped into the beaker with the denatured collagen suspension (at a flow rate of approximately 10 mL/min) and allowed to sit without disturbing for 1 hour. After this time, a 50 mL pipet was used to remove the clear supernatant leaving in the beaker only the gelatinous collagen gel and 600 mL of 0.6% (v/v) acetic acid solution was added to the 4 L beaker containing collagen and stirred overnight at 4° C. in a cold room. On the next day, the collagen suspension was transferred into sterile dialysis tubbing (7KDa MWCO) and dialyzed against DI water in 4 L beakers to remove excess salts for 4 days changing the water every 12 hours. Finally, the dialyzed collagen suspension was freeze-dried in a 1 L benchtop lyophilizer from LABCONCO for over 48 hours, and the dried collagen was stored refrigerated (4 to 8° C.) until use.

PLECM Decellularization from a Porcine Liver

PLECM extraction from porcine livers followed ECM extraction procedures described previously in the literature, with modifications as described below. The porcine liver was obtained from Friendly Nick's Butcher (Fort Collins, CO), cut into smaller pieces, and kept frozen in a −80° C. freezer until the decellularization.

In a biosafety cabinet, the porcine liver was sliced into small square pieces (˜3 mm) with the aid of a sterile knife/slicing tool and placed into a sterile 4 L beaker filled with sterile DI water. The mixture was vigorously stirred at 4° C. in a cold room and the DI water was replaced every hour. This step was repeated about 6 times (total 12 hours) until no more blood was visible in the water. A sterile collander/mesh was used to retain the pieces of tissue while replacing the water. After 6 times, the water was removed and 0.1% (w/v) sodium dodecyl sulfate (SDS) solution was added to the container with the tissue pieces and allowed to stir overnight at 4° C. in a cold room for decellularization of the ECM. If all the decellularized ECM pieces showed no presence of trapped blood, the SDS solution was removed and the ECM was stirred with DI water, replacing the DI water every hour and repeating this step about 4 times to remove the SDS. When trapped blood could be seen in the ECM pieces, the process was restarted in the biosafety cabinet to cut the decellularized ECM into smaller pieces. After removing the residual SDS, the DI water was removed and the beaker was filled with 70% ethanol and stirred overnight at 4° C. in a cold room. On the next day, in a biosafety cabinet, the ethanol was removed and the ECM was separated into 50 mL conical tubes up to 20 mL, exposing as much as ECM surface as possible and was freeze-dried in a 1 L benchtop lyophilizer from LABCONCO over 48 hours. The lyophilized PLECM (1 g) was dissolved in 100 mL of 0.1 M HCl containing 100 mg of >2500 mg/units pepsin (9:1) overnight under constant stirring. The suspension was centrifuged at 8000 g for 10 minutes, the supernatant was retrieved and dialyzed (7KDa MWCO) against 0.01 M HCl solution in a 4 L beaker for 3 days, exchanging the dialysis solution twice a day, and followed by dialysis against water for 3 more days, exchanging the dialysis solution twice a day, or until the pH of the deionized water reached 5.0. Finally, the dialyzed PLECM suspension was freeze-dried in a 1 L benchtop lyophilizer from LABCONCO over 48 hours and the dried PLECM was stored in the fridge (4 to 8° C.) until use.

### Fabrication of ECM Fiber

Procedures for collagen I extraction from rat tails and porcine liver decellularization are detailed above and process of porcine liver decellularization are provided in FIG. 1. The solvent 1,1,1,3,3,3-hexafluoro-2-propanol (HFIP) was used to dissolve collagen and PLECM blends overnight under constant stirring, while trifluoroacetic acid (TFA) was used to dissolve chitosan-only solutions; concentrations are provided in Table 1. Viscosity measurements of the solutions were taken with a TL7 spindle on a Fungilab (Hauppauge, NY) viscometer at 100 rpm. A glass syringe (Fortuna Optima, Luer lock tip style) containing the macromolecule solution was then placed in a Kent Scientific Genie Plus syringe pump (Torrington, CT) and the solutions were pumped (0.5-1.0 mL h⁻¹) for 5+ h. A high-voltage DC power supply (operated at 15-20 kV) (Gamma High Voltage Research, Ormond Beach, FL) was used to create an electric field between a 19-gauge needle and a grounded copper collection plate covered with aluminum foil. Siliconized round glass coverslips (12 mm diameter, Hampton research, Aliso Viejo, CA) were oxidized via oxygen plasma for 5 min to facilitate nanofiber attachment and then attached to the aluminum foil using copper tape. The distance of the tip of the needle to the siliconized round glass cover slides was fixed at 15 cm for all conditions. Electrospinning was then conducted at 20±2° C. and 19% relative humidity; viscosities and electrospinning conditions for each macromolecule solution are provided in Table 2.

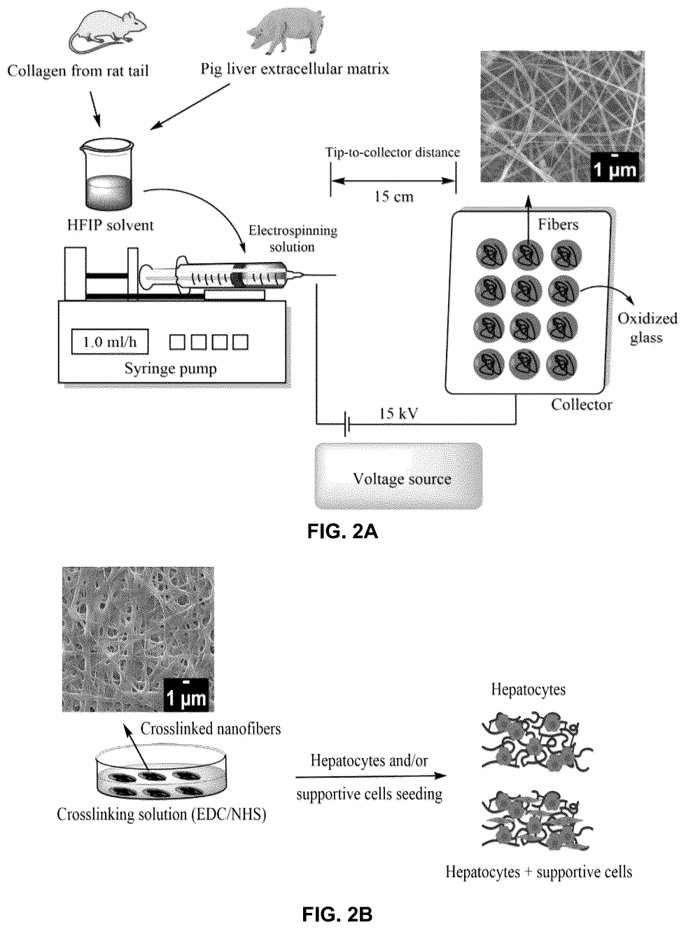

Nanofibers that were adhered to the coverslips were crosslinked at room temperature (RT) for 24 h using a mixture of 1-ethyl-3-(3-dimethyl aminopropyl)carbodiimide (EDC, 20 mm) and N-hydroxysuccinimide (NHS, 10 mm) in 90% ethanol for all nanofibers except for the chitosan-only nanofibers that were crosslinked with a vapor of 25% glutaraldehyde in water. Finally, the nanofibers were washed several times with sterile deionized water (obtained from a Milli-Q water purification system) and kept at 4° C. until further use. The process described above is shown schematically in FIG. 2A-2B.

To generate control substrates, O₂ plasma-treated glass coverslips were incubated for 2 h in 100 μg mL⁻¹ of either collagen I dissolved in 0.01 N acetic acid or PLECM dissolved in 0.02 N hydrochloric acid. Following two rinses with double-distilled water (ddH₂O), the coverslips were sterilized with 70% v/v ethanol in ddH₂O for 1 h and finally rinsed three times with sterile ddH₂O.

TABLE 1
Concentration of macromolecules in solutions
used for each fiber condition

| | Collagen | PLECM | Chitosan |
|---|---|---|---|
| Sample | (% w/v) | (% w/v) | (% w/v) |
| Collagen | 5 | — | — |
| Collagen/Chitosan | 5 | — | 2 |
| Collagen/PLECM | 5 | 2 | — |
| PLECM | — | 2 | — |
| PLECM/Chitosan | — | 2 | 2 |
| Chitosan | — | — | 2 |
| PLECM microfibers | — | 5 | — |

TABLE 2
Electrospinning parameters and diameter range
of uncrosslinked and crosslinked nanofibers

| | Distance (cm)/ | | |
|---|---|---|---|
| | voltage (kV)/ | Diameter - | Diameter - |
| | flow rate | uncrosslinked | crosslinked |
| Sample | (mL · h⁻¹⁾ | (nm) | (nm) |
| Collagen | 15/15/1.0 | 187 ± 86 | 165 ± 70 |
| Collagen/Chitosan | 15/17/0.5 | 125 ± 52 | 131 ± 51 |
| Collagen/PLECM | 15/15/1.0 | 223 ± 129 | 192 ± 83 |
| PLECM | 15/20/1.0 | 211 ± 130 | 241 ± 133 |
| PLECM/Chitosan | 15/17/0.5 | 146 ± 56 | 149 ± 68 |
| Chitosan | 15/17/0.5 | 175 ± 87 | 178 ± 85 |
| PLECM microfibers | 15/20/1.0 | 932 ± 558 | 1100 ± 718 |

### Characterization of ECM Fiber

Nanofibers were characterized via Fourier transform infrared spectroscopy using attenuated total reflection (FTIR-ATR, Thermo Smart Orbit mounted on a Thermo 8700 spectrometer) with a diamond crystal using a wavelength range of 4000-500 at 4 cm⁻¹ resolution and cumulation of 64 scans. The spectra of the nanofibers were compared to the spectra of their pure components. Additionally, nanofibers were coated with 15 nm of gold and observed using a JSM-6500F JEOL Scanning Electron Microscope (SEM, Tokyo, Japan) at an accelerating voltage of 15 kV. The chemical composition of the nanofibers was characterized by X-ray photoelectron spectroscopy (XPS, 5800 spectrometer, Physical Electronics, Chanhassen, MN). Survey spectra were collected for all nanofibers from 0 to 1100 eV, with a pass energy of 187 eV. High-resolution spectra of the C1s peak were acquired with 0.1 eV steps and an X-ray spot of 800 μm. Origin and Multipak software were used for performing the curve fitting of all presented spectra. The C1S peak (284.8 eV) was used as a reference peak. Nanofiber spectra were compared to the spectra of their respective pure components. Porosity and pore size of the fibers were measured from the SEM images with the aid of ImageJ DiameterJ software using 5 different pictures and the results are shown in Table 3. Mechanical properties of the nanofibers were measured using an Instron 4442 (Norwood, MA) tensile tester (n≥3) and results are shown in Table 4.

TABLE 3
Porosity and pore size range of uncrosslinked and crosslinked nanofibers

| | Uncrosslinked | | Crosslinked | |
|---|---|---|---|---|
| Sample | Porosity (%) | Pore area (μm²) | Porosity (%) | Pore area (μm²) |
| Collagen | 50.02 ± 12.14 | 0.39 ± 0.58 | 59.57 ± 8.88 | 0.27 ± 1.00 |
| Collagen/Chitosan | 50.22 ± 4.87 | 0.27 ± 0.50 | 57.97 ± 4.69 | 0.15 ± 1.12 |
| Collagen/PLECM | 45.79 ± 5.12 | 0.58 ± 0.83 | 52.94 ± 4.75 | 0.65 ± 1.39 |
| PLECM | 47.40 ± 1.66 | 2.35 ± 3.42 | 53.89 ± 2.68 | 0.39 ± 3.06 |
| PLECM/Chitosan | 57.55 ± 11.46 | 0.30 ± 0.44 | 51.27 ± 7.86 | 0.28 ± 1.39 |
| Chitosan | 49.20 ± 2.07 | 0.38 ± 0.51 | 45.88 ± 4.81 | 0.30 ± 0.40 |
| PLECM microfibers | 46.20 ± 4.28 | 8.11 ± 11.12 | 56.60 ± 14.73 | 8.07 ± 9.50 |

TABLE 4
Mechanical properties of crosslinked nanofibers

| | Tensile strain | Maximum tensile | Young's |
|---|---|---|---|
| | at break | stress | modulus |
| Sample | (%) | (MPa) | (MPa) |
| Collagen | 23.34 ± 1.18 | 2.42 ± 1.32 | 0.78 ± 0.38 |
| Collagen/Chitosan | 20.04 ± 1.71 | 1.01 ± 0.09 | 0.34 ± 0.05 |
| Collagen/PLECM | 17.43 ± 2.12 | 0.17 ± 0.03 | 0.11 ± 0.04 |
| PLECM | 12.82 ± 3.14 | 0.82 ± 0.24 | 0.28 ± 0.15 |
| PLECM/Chitosan | 22.65 ± 0.64 | 1.24 ± 0.24 | 0.38 ± 0.10 |
| Chitosan | 24.97 ± 2.22 | 5.18 ± 0.56 | 1.38 ± 0.31 |
| PLECM microfibers | 11.08 ± 2.95 | 1.55 ± 0.59 | 1.12 ± 0.53 |

### NPC Culture

Primary human liver sinusoidal endothelial cells (LSECs, Cell Systems, Kirkland, WA) were passaged up to 14 times using 0.05% trypsin-EDTA (Corning, Manassas, VA) in tissue culture flasks coated with 2 μg cm⁻² fibronectin (Corning); cells were cultured in EGM-2 Endothelial Cell Growth Medium-2 BulletKit (Lonza, Williamsport, PA). The 3T3-J2 murine embryonic fibroblasts, a gift from Howard Green (Harvard Medical School), were passaged up to 11 times using 0.25% trypsin-EDTA (Corning) in tissue culture flasks with medium containing high glucose Dulbecco's Modified Eagle Medium (DMEM) base (Corning) containing 10% v/v bovine calf serum and 1% v/v penicillin-streptomycin (Corning).

### PHH Monocultures and PHH/NPC Cocultures

Nanofibers adhered to coverslips and adsorbed ECM control coverslips were rinsed once with 1× phosphate buffered saline (PBS, Corning) and incubated overnight in phenol red-free high glucose DMEM (Cytiva, Marlborough, MA) containing 10% bovine calf serum and 1% penicillin-streptomycin. The nanofibers and ECM controls were then transferred to the wells of a 24-well polystyrene plate precoated with 5% m/v Pluronic F-127 (Sigma-Aldrich, Saint Louis, MO) to prevent cell attachment to the polystyrene. Cryopreserved PHHs (donor lots HUM4192, 16 year old Asian female, and HUM4055C, 54 year old Caucasian female) from Lonza (Walkersville, MD) were thawed, their viability was assessed using the Trypan Blue dye exclusion method, and then PHHs were seeded onto the above substrates at 200 000 cells in 500 μL per well of seeding medium containing phenol red-free high glucose DMEM with 10% fetal bovine serum (R&D Systems, Minneapolis, MN), 1.5% v/v N-2-hydroxyethylpiperazine-N′-2-ethanesulfonic acid (HEPES, Corning) buffer, 1% v/v insulin-transferrin-selenium-linoleic acid (ITS+) premix (Corning), 1% penicillin/streptomycin, 100 nm dexamethasone (Sigma-Aldrich), and 7 ng mL⁻¹ glucagon (Sigma-Aldrich). The cultures were washed the next day once with phenol red-free DMEM once to remove unattached cells and the culture medium was replaced with maintenance medium (similar recipe as seeding medium above but with 10% bovine calf serum). To create cocultures, 100 000 3T3-J2s or LSECs in 500 μL per well were seeded onto PHH-laden nanofibers. For PHH-LSEC cocultures, 20 ng mL⁻¹ of human recombinant vascular endothelial growth factor (VEGF)-165 protein (Invitrogen, Waltham, MA) was added to the maintenance medium to maintain endothelial cell viability. The medium was replaced on all cultures every 2 days.

### Cell Viability and Functional Assessments

PrestoBlue (ThermoFisher, Waltham, MA) was used to assess cell viability/health according to manufacturer's protocol. Albumin in supernatants was measured using a sandwich-based enzyme linked immunosorbent assay (ELISA, Bethyl Laboratories, Montgomery, TX) with horseradish peroxidase detection and 3,3′5,5′-tetramethylbenzidine substrate (TMB, Rockland Immunochemicals, Boyertown, PA). Urea in supernatants was measured using diacetyl monoxime with acid and heat (Stanbio Labs, Boerne, TX). Absorbance of the samples was read on the Synergy H1 multimode plate reader (Biotech, Winooski, VT).

CYP 3A4 and 2C9 enzyme activities were measured by incubating the cultures for 3 h with luciferin-IPA (Promega Life Sciences, Madison, WI) or luciferin-H (Promega), respectively, followed by the processing of collected supernatants per manufacturer's recommendations; luminescence was quantified with the Synergy H1 multimode reader. CYP1A2 and CYP2A6 enzymatic activities were measured by incubating the cultures for 3 h with 5 μm 7-ethoxyresorufin (Sigma-Aldrich) or 50 μm coumarin (Sigma-Aldrich), respectively. The CYP2A6-generated metabolite, 7-hydroxycoumarin (7-HC), and CYP1A2-generated metabolite, resorufin, were quantified using fluorescence measurements (excitation/emission 355/460 nm for 7-HC and 550/585 nm for resorufin) on the Synergy H1 multimode reader.

### Microscopy for Cell Visualization

Cultures were fixed with 4% v/v paraformaldehyde (PFA, Alfa Aesar, Wand Hill, MA) in ddH₂O for 20 min, rinsed three times with 1× PBS, and then incubated for 45 min at RT with a blocking solution containing 5% v/v donkey serum (Southern Biotech, Birmingham, AL) and 0.3% v/v Triton X-100 (Ameresco, Solon, OH) in 1× PBS. Goat antihuman albumin (Abcam, Cambridge, MA), rabbit antihuman cytokeratin 8 (CK8, Invitrogen), rabbit antihuman asialoglycoprotein receptor 1 (ASGR1, Proteintech, Rosemont, IL), mouse antihuman multidrug resistance-associated protein 2 (MRP2, Santa Cruz, Dallas, TX), mouse antihuman CD31 (Cell Signaling Technology, Danvers, MA), and mouse antihuman alpha-smooth muscle actin (αSMA) (R&D Systems) primary antibodies were diluted at 1:200 in dilution solution containing 0.1% m/v bovine serum albumin (Sigma-Aldrich) and 0.3% Triton X-100 in 1× PBS and incubated on the cultures at 4° C. overnight. Cultures were rinsed the next day with 1× PBS three times and then incubated with 1:100 diluted secondary antibodies, donkey antigoat (Alexa Fluor 488, green), donkey anti-mouse (Alexa Fluor 647, Cy5) (Invitrogen), and donkey antirabbit (Alexa Fluor 568, red) for 1 h at RT. DAPI (4′,6-diamidino-2-phenylindole, MP Biomedicals, Solon, OH) at 300 nm concentration was added to the cultures for the last 15 min of the incubation period. After incubation, the cultures were rinsed with 1× PBS three times and observed under confocal microscopy with the filters of 638-755 nm for Cy5 and 410-488 nm for Alexa Fluor 488 and DAPI (Zeiss LSM 710, Zeiss, Germany; Olympus/Evident Scientific FV3000, Singapore). Maximum intensity images were obtained using ZEISS ZEN microscope software (Zeiss, Germany) and ImageJ.

For SEM, cultures were fixed for 45 min at RT with 3% v/v glutaraldehyde in ddH₂O containing 0.1 m sucrose and 0.1 m sodium cacodylate (Sigma-Aldrich), followed by incubation for 10 min in a solution containing 0.1 m sodium cacodylate and 0.1 m sucrose. The samples were then dehydrated by adding increased concentrations of ethanol (35%, 50%, 70%, 100%, respectively) for 10 min each. Finally, the samples were sputter coated with gold (15 nm) and imaged using the JSM-6500F JEOL SEM at an accelerating voltage of 15 kV.

### Data Analysis

Experiments were repeated 2-3 times with two PHH donors. Data analysis and visualization were performed using Microsoft Excel and GraphPad Prism (La Jolla, CA). Error bars represent standard deviation (2-3 technical replicates from a representative experiment). Statistical significance was determined via 2-way analysis of variance (ANOVA) with Tukey's or Dunnett's multiple comparisons tests.

### Example 1. Morphological and Mechanical Characterization of the ECM Fibers

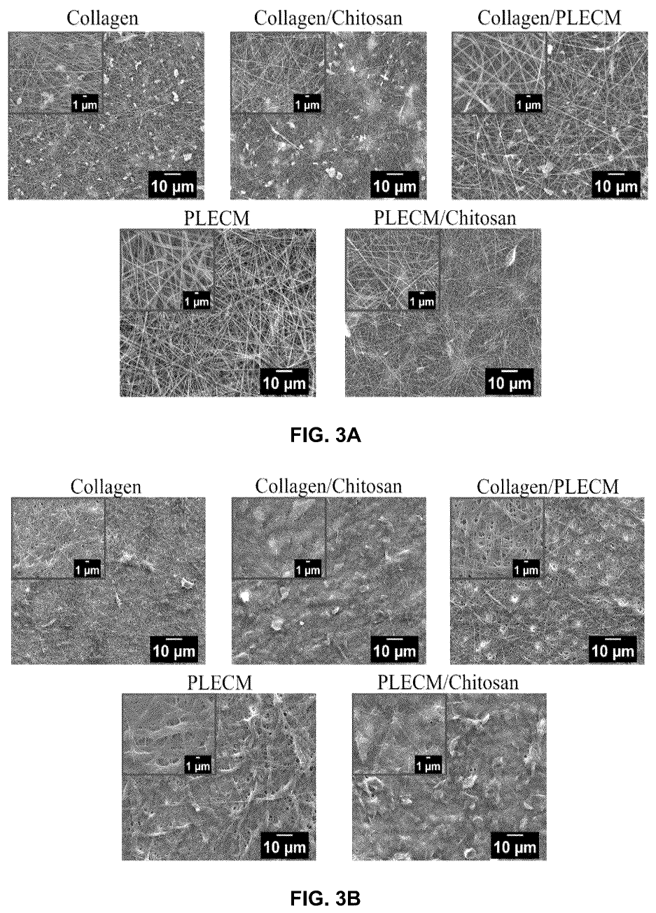

Nanofibers composed of collagen (type 1), PLECM, and their blends with chitosan were successfully electrospun. Uncrosslinked nanofibers displayed high porosity, a random orientation, and no beads were detected, though uncrosslinked nanofibers contained small particles in their structure, potentially due to the presence of insoluble fatty acids (FIG. 3A). Average porosities of uncrosslinked nanofibers ranged from ≈46% (collagen/PLECM) to ≈58% (PLECM/chitosan), but were statistically similar (Table 3). Average pore areas of uncrosslinked nanofibers ranged from approximately 0.3 μm² (collagen/chitosan and PLECM/chitosan) to approximately 0.6 μm² (collagen/PLECM) with the exception being PLECM that displayed an average pore area of approximately 2.3 μm²; however, pore areas for all nanofibers were statistically similar given the variations observed (Table 3).

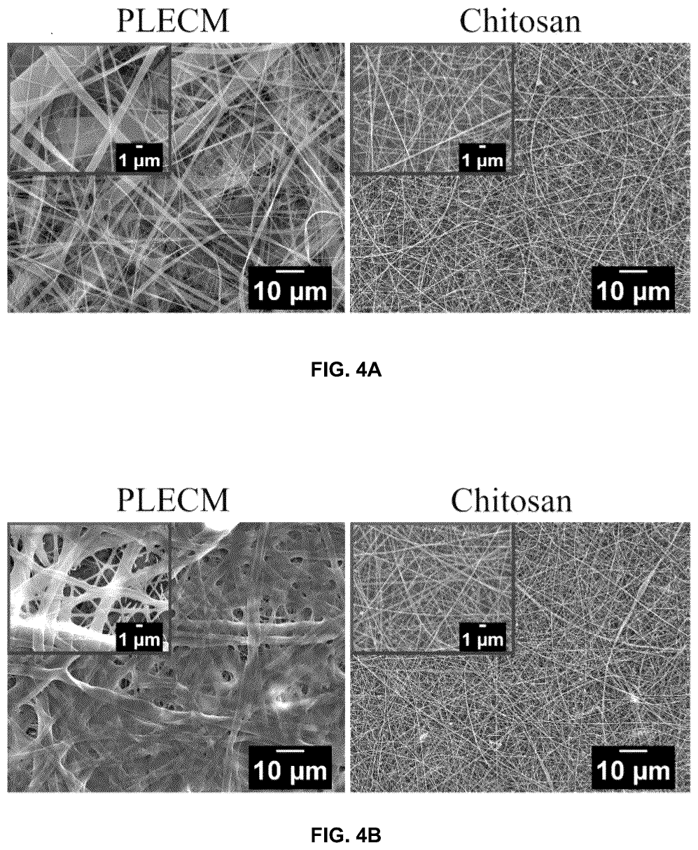

Following crosslinking, ECM nanofibers maintained their morphology (FIG. 3B) and average porosities (≈46-60%) but the average pore areas were generally lower for the crosslinked nanofibers (≈0.15-0.6 μm²) as compared to the non-crosslinked nanofibers (approximately 0.3-2.3 μm²) (Table 3) due to swelling of the nanofibers after crosslinking. Following crosslinking, nanofibers containing chitosan showed agglomerates of fibers like beads (FIG. 3B). Interestingly, the above-mentioned small particles present in the un-crosslinked collagen nanofibers appeared to have been removed following the washes of the crosslinking process. Chitosan-only nanofibers were crosslinked by glutaraldehyde vapor to avoid high swelling and loss of nanofiber morphology, which occurred in the 90% ethanol solution with EDC/NHS that was used to crosslink nanofibers containing collagen and/or PLECM. Furthermore, the ECM nanofibers displayed statistically similar nanoscale diameters before and after crosslinking (Table 4). Finally, chitosan-only nanofibers retained their morphology, diameters, and porosity following crosslinking, while 5% w/v PLECM-only solutions generated microfibers instead of nanofibers (FIGS. 4A-4B).

The average Young's moduli for the fibers ranged from approximately 0.1 MPa (collagen/PLECM) to approximately 1.4 MPa (chitosan) (Table 4). Along with collagen/PLECM nanofibers, the other nanofibers with lower average Young's moduli included PLECM (approximately 0.3 MPa), collagen/chitosan (approximately 0.3 MPa), and PLECM/chitosan (approximately 0.4 MPa); in contrast, collagen nanofibers (approximately 0.8 MPa), chitosan nanofibers (approximately 1.4 MPa), and PLECM microfibers (approximately 1.1 MPa) displayed higher average Young's moduli. Analogously, the maximum tensile stress was lowest for the softer nanofibers (collagen/PLECM approximately 0.2 MPa, PLECM approximately 0.8 MPa, collagen/chitosan approximately 1 MPa, and PLECM/chitosan approximately 1.2 MPa) and higher for the stiffer fibers (collagen nanofibers approximately 2.4 MPa, chitosan nanofibers approximately 5 MPa, and PLECM microfibers approximately 1.6 MPa). Finally, the elongation at break of the fibers (i.e., tensile strain) were similar (approximately 17-25%) except for PLECM-only nanofibers and microfibers, which had the lowest percentage strain of approximately 11% to approximately 13% (Table 4).

### Example 2: Biochemical Characterization of the ECM Fibers

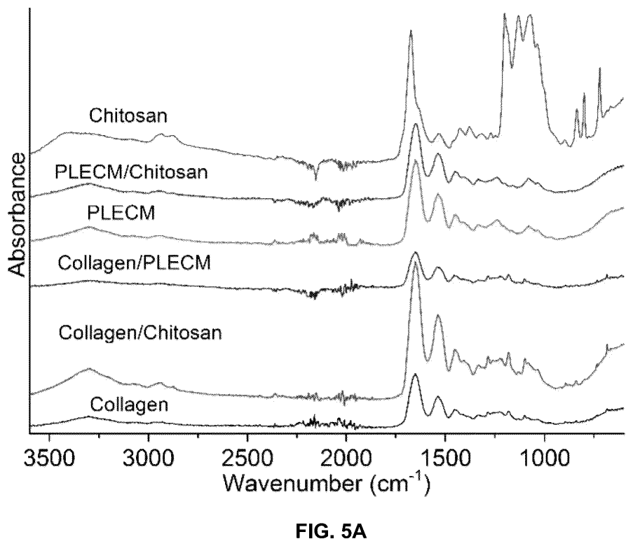

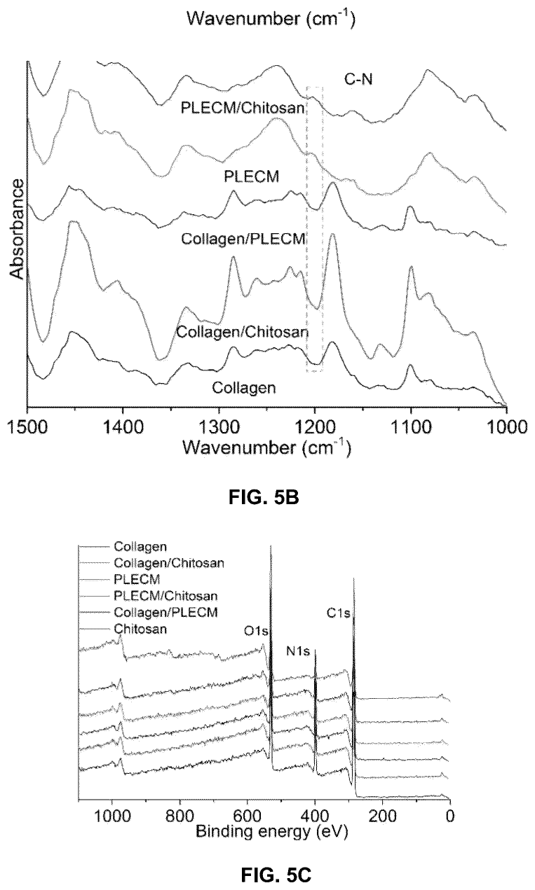

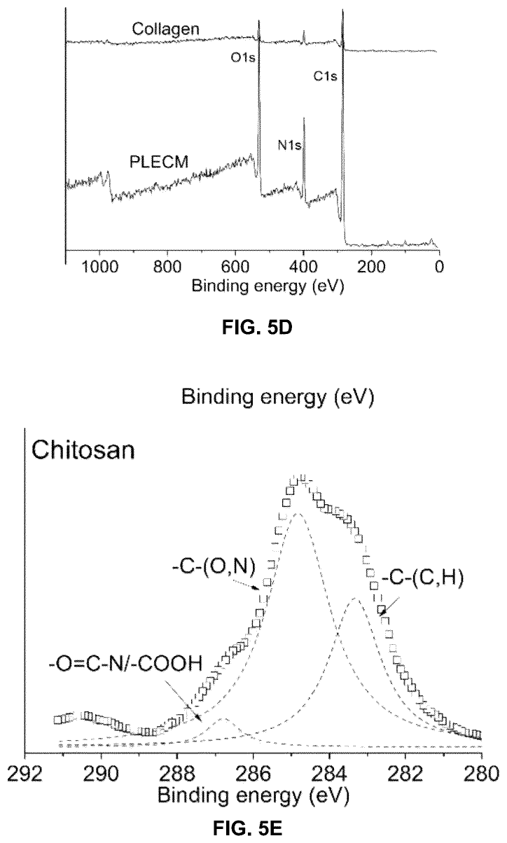

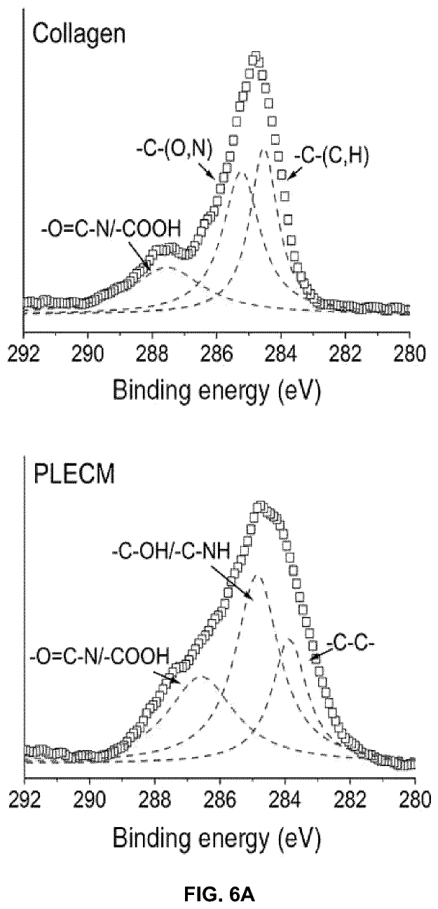

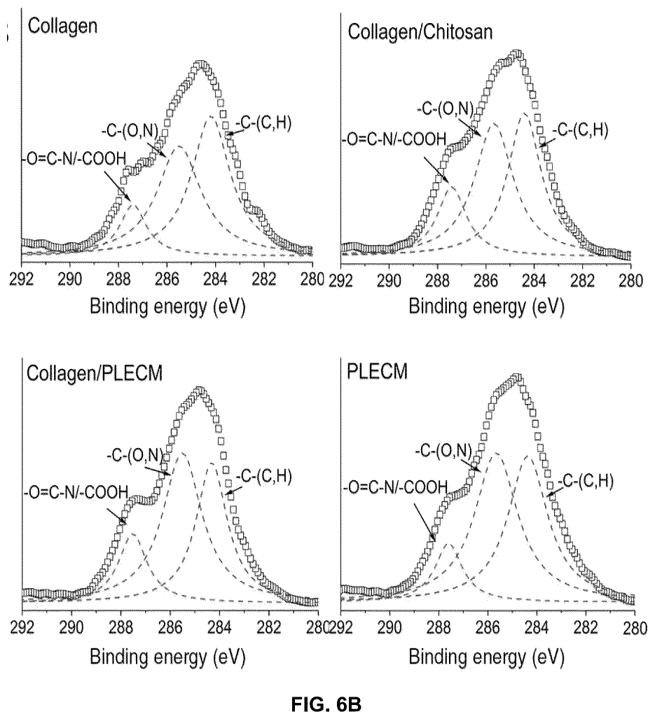

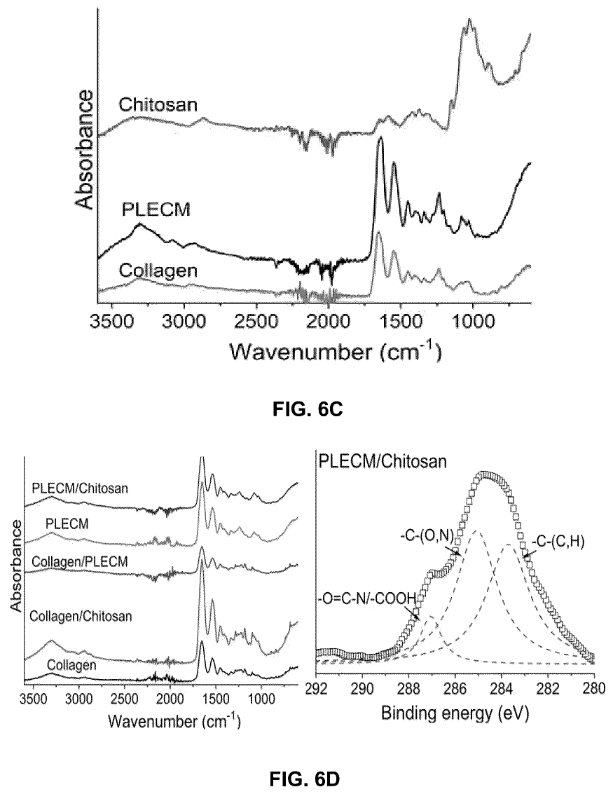

XPS survey spectra for the ECM nanofibers and the individual constituent macromolecules showed peaks from carbon (C1s), oxygen (O1s), and nitrogen (N1s) but at different ratios (FIG. 5A-5B). High-resolution XPS C1s spectra from all nanofibers showed peaks at approximately 287.5, 285.5, and 284 eV related to O═N—C and COOH groups, C—(O, N) groups, and C—(C, H) groups, respectively, with some shifting on the binding energy (eV) and differences on the area/intensity of the peaks (FIG. 6B; and FIG. 5E). The relative intensity of the C—N groups compared to aliphatic carbon was higher in PLECM, likely due to a different protein composition than collagen alone (FIG. 6A). Overall, the XPS data confirm that the macromolecules were incorporated into their respective nanofibers.

The FTIR spectra of the nanofibers containing collagen and/or PLECM showed similar peaks, likely because collagens are the majority components within PLECM. Characteristic absorptions were observed at approximately 3300 cm⁻¹ (N—H stretching, amide A), approximately 3060 cm⁻¹ (C—H stretching, amide B), approximately 1650 cm⁻¹ (C═O stretching, amide I), approximately 1530 cm⁻¹ (N—H deformation, amide II), and approximately 1280 cm⁻¹ (coupled C—N stretching and N—H bending, amide III) (FIG. 6D). Interestingly, the amide Ill peak at approximately 1280 cm⁻¹ could only be observed in the collagen nanofiber spectra but not in the PLECM nanofiber spectra, whereas a peak at approximately 1200 cm⁻¹ (C—N stretching) could only be observed on the PLECM but not on the collagen nanofiber spectra (FIG. 5B), suggesting that there are some structural differences across the two ECM types. Finally, all features of the FTIR spectra of the nanofibers (FIG. 6D) were present in the FTIR spectra of the corresponding individual macromolecules (FIG. 6C).

### Example 3: Effects of ECM Nanofibers on the Morphology and Secretory Functions of PHH Monocultures

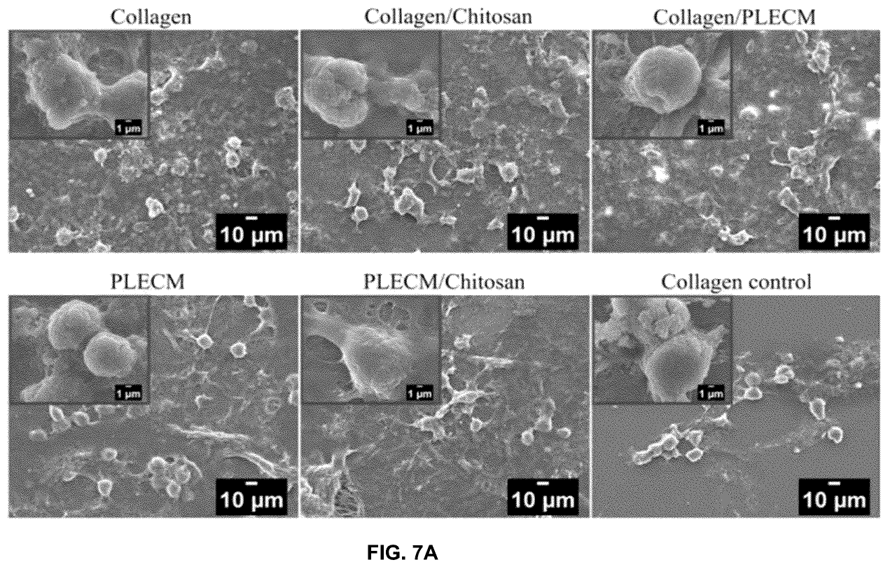

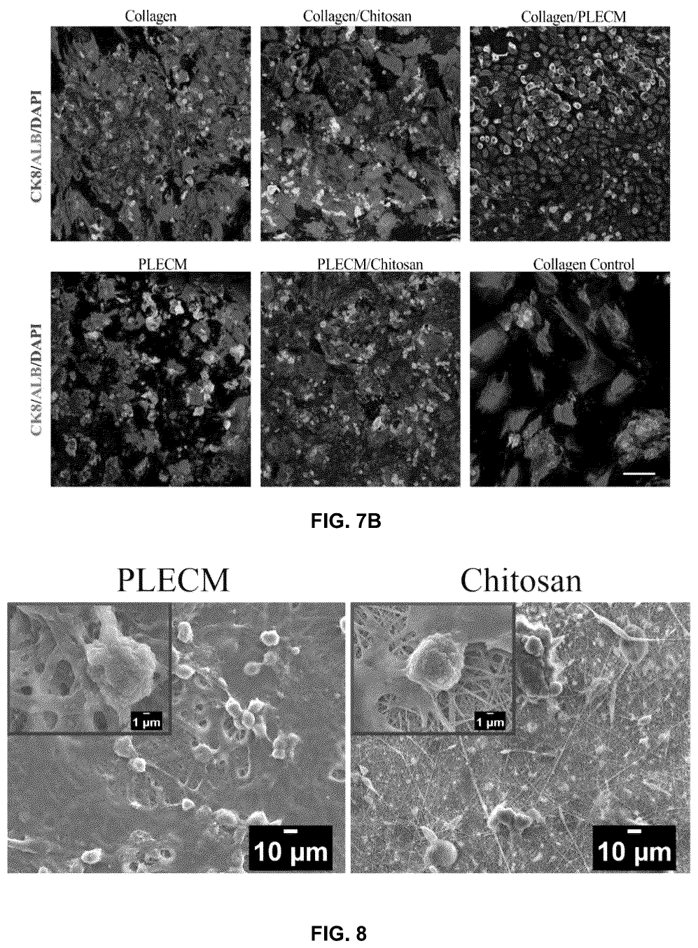

PHHs maintained similar morphology for 23 days on nanofibers containing collagen and PLECM and adsorbed ECM control substrates as assessed via SEM, albeit fewer PH Hs were observed on the adsorbed ECM controls, which was expected due to the typical loss of PHH viability/numbers on conventional (adsorbed ECM) stiff substrates. Furthermore, PHHs appeared to have infiltrated into the nanofiber layers while maintaining their contacts with other neighboring cells (FIG. 7A), whereas chitosan-only nanofibers resulted in lower PHH attachment and no apparent infiltration of PHHs into the nanofiber layers (FIG. 8).

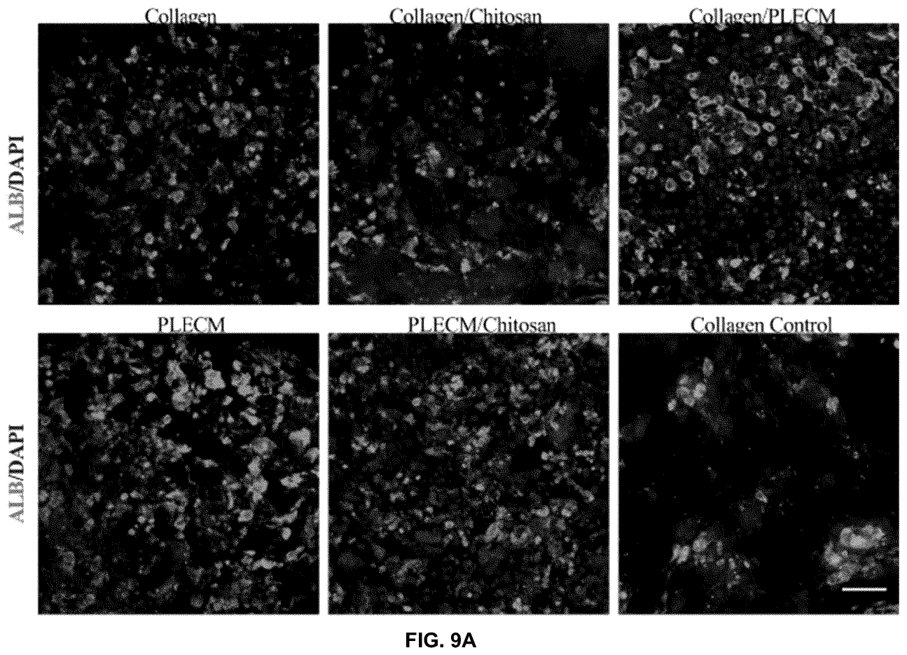

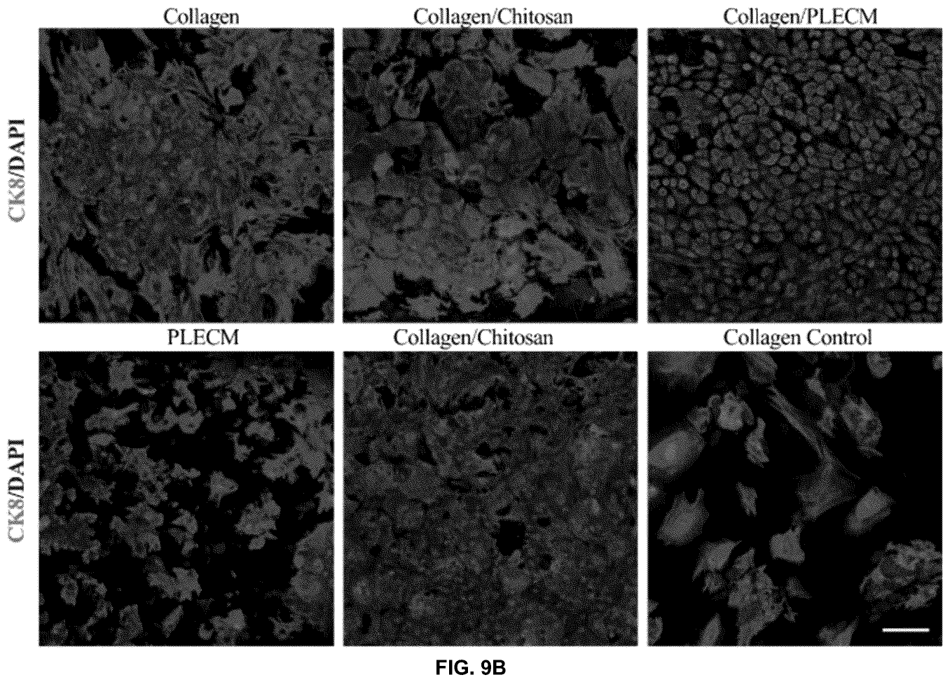

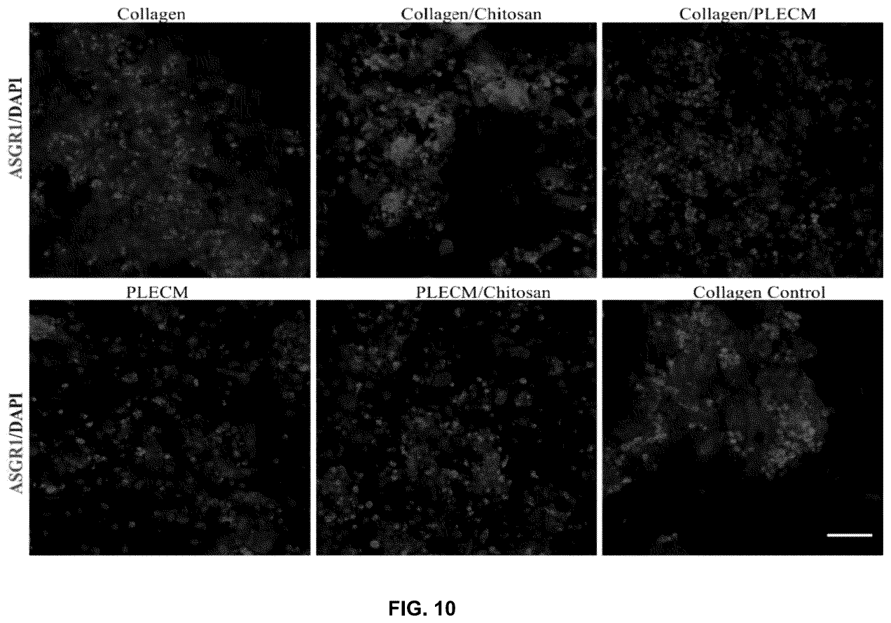

Coimmunostaining for human albumin and human CK8 showed mostly single, sparsely distributed, and spread-out PHHs on the adsorbed ECM controls; in contrast, a larger number of PHHs were observed on the nanofibers, albeit PHH spreading patterns varied across the nanofibers, with PLECM and collagen/PLECM nanofibers containing less spread-out PHHs (FIG. 7B), which is likely due to the lower Young's moduli of the PLECM (approximately 0.3 MPa) and collagen/PLECM (approximately 0.1 MPa) nanofibers allowing the PHHs to remain more spheroidal due to fewer traction forces as also seen on other soft substrates like gels composed of Matrigel. Interestingly, while some PHHs displayed both albumin and CK8 staining, many spread-out PHHs only had detectable CK8 staining (FIG. 7B; and FIGS. 9A-9B) suggesting the loss of specialized liver functions (i.e., de-differentiation). Finally, we also detected ASGR1 (hepato-specific marker) on PH Hs cultured on nanofibers and adsorbed controls; while there were more ASGR1 positive PHHs on the nanofibers than the adsorbed controls, the expression of ASGR1 varied considerably across individual PHHs (FIG. 10), which may be due to the variability in the extent/number of homotypic interactions in the local microenvironment of the cells.

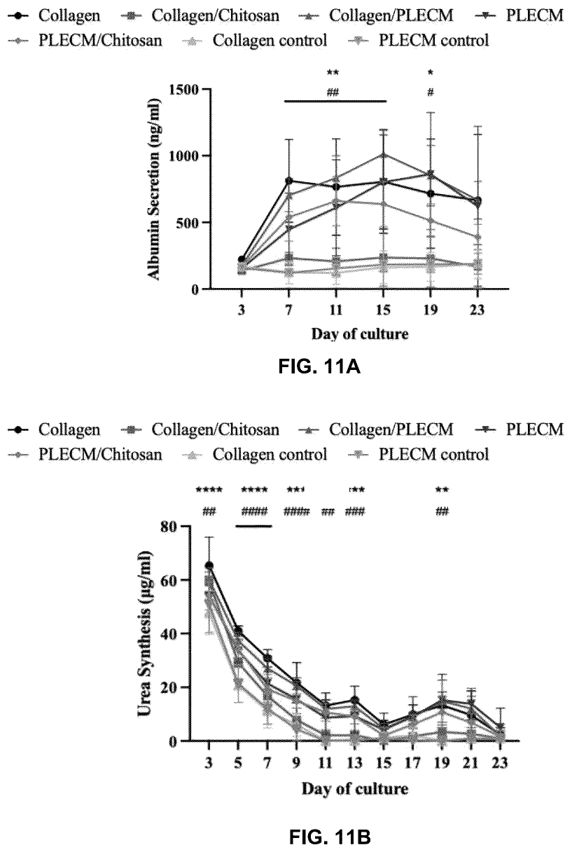

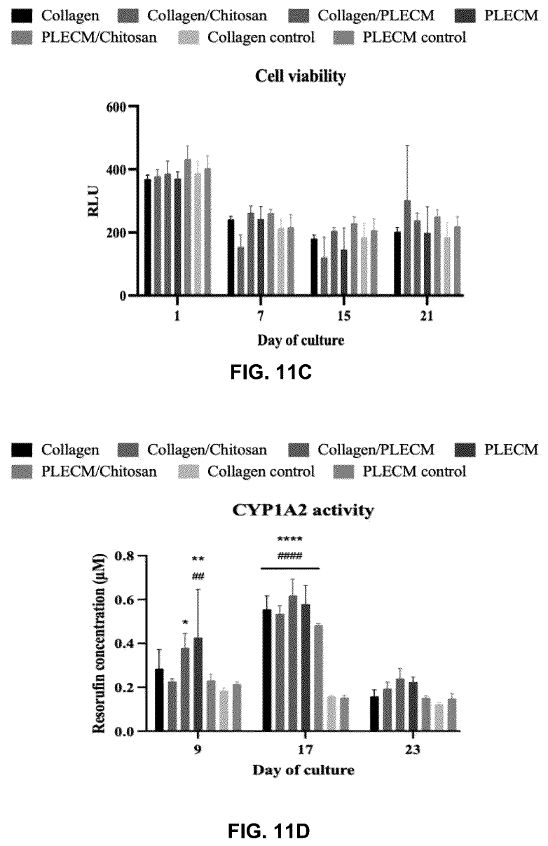

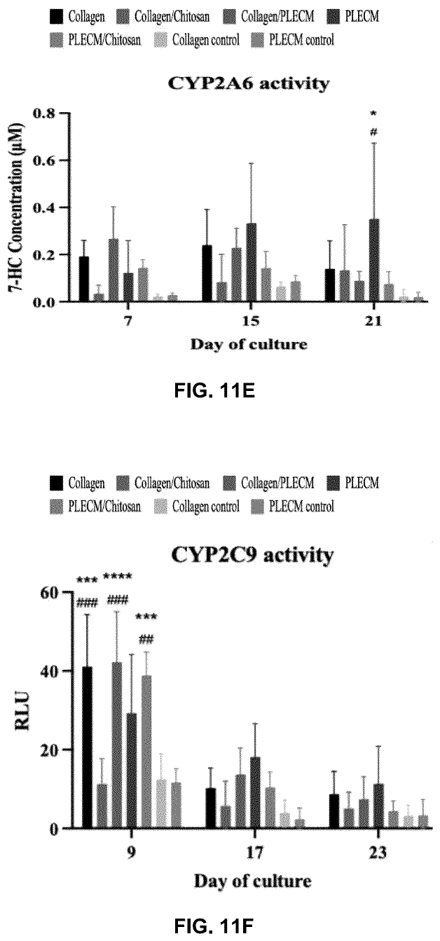

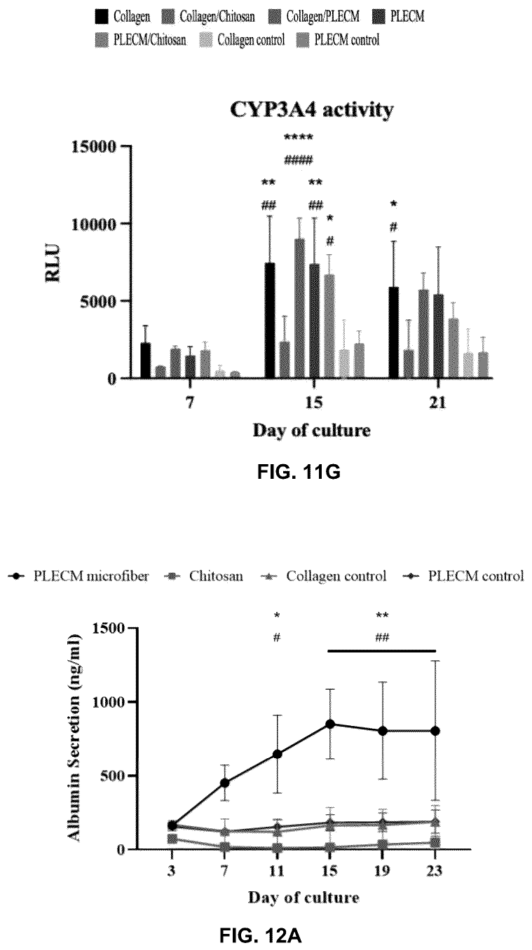

PHH overall viability/health was similar over time across ECM nanofibers and adsorbed ECM controls, albeit values decreased by 20-40% after the first day of culture (FIG. 11C), likely due to some cell detachment over the first week of culture as is common with other PHH monoculture models.

PHHs cultured on the adsorbed ECM controls secreted low but relatively stable levels of albumin (a widely utilized surrogate marker of liver protein synthesis) over 23 days, whereas albumin secretion rates on the ECM nanofibers increased over the first week of culture, remained relatively stable for 19 days, and then declined 7-30% by day 23 of culture; furthermore, the maximal albumin secretion rates were 5.4-fold higher on the collagen/PLECM nanofibers, 4.6-fold higher on the PLECM nanofibers, 4.2-fold higher on the collagen nanofibers, 3.5-fold higher on the PLECM/chitosan nanofibers, and 1.3-fold higher on the collagen/chitosan nanofibers as compared to the adsorbed ECM controls (FIG. 11A). Higher albumin secretion rates have been correlated with a higher differentiation status of PHHs, suggesting that the nanofibers better maintain PHH differentiated phenotype than adsorbed (conventional) ECM substrates. Finally, the albumin secretion rates did not always correlate with the immunostaining pictures for albumin staining. For instance, staining patterns showed lower albumin expression in PHHs on collagen nanofibers versus PLECM nanofibers after 23 days of culture (FIGS. 9A-9B), whereas albumin secretion rates were similar at the same time-point (FIG. 11A). While it is difficult to compare qualitative observations via images with quantitative secretion data, such disparities across the two outcomes may be related to longer retention of albumin in the cytoplasm as opposed to its transport/secretion into the supernatants; regardless, the secretion of albumin is the relevant and critical physiologic function that is typically measured to appraise the functionality/quality of in vitro liver platforms.

In contrast to albumin secretion, PHH urea synthesis (a widely utilized marker of liver ammonia detoxification) rates displayed a continuous decline over time in all cultures (nanofibers and adsorbed ECM control substrates) and after 23 days had decreased by 90-98% of day 1 levels; however, urea synthesis rates were still 2.5-fold higher on the collagen nanofibers, 1.03-fold higher on the collagen/chitosan nanofibers, 1.8-fold higher on the collagen/PLECM nanofibers, 5.3-fold higher on the PLECM nanofibers, and 1.7-fold higher on the PLECM/chitosan nanofibers as compared to the adsorbed ECM controls after 23 days of culture (FIG. 11B). Urea synthesis involves the coordinated action of several enzymes of the urea cycle and is typically difficult to maintain at steady levels in PHH monocultures even with various ECM combinations/manipulations; therefore, it was not surprising here that nanofibers upregulated but did not stabilize urea synthesis in PHH monocultures as compared to adsorbed ECM substrates.

### Example 4: Effects of ECM Nanofibers on the CYP Enzyme Activities of PHH Monocultures

CYP enzymes are involved in the metabolism/biotransformation of most xenobiotics including 70-80% of all drugs in clinical use; the highest expressed forms in liver are CYPs 3A4, 2C9, 2C8, 2E1, and 1A2, while 2A6, 2D6, 2B6, 2C19, and 3A5 are less abundant. Therefore, the activities of less abundant (CYP2A6) and more abundant (CYPA2, 2C9, 3A4) CYP enzymes were measured in PHHs on nanofibers and adsorbed ECM controls using well established fluorescent/luminescent high-throughput assays.

Maximal PHH CYP1A2 activities were 2.8-fold higher on the collagen nanofibers, 2.7-fold higher on the collagen/chitosan nanofibers, 3.1-fold higher on the collagen/PLECM nanofibers, 2.9-fold higher on the PLECM nanofibers, and 2.4-fold higher on the PLECM/chitosan nanofibers as compared to the adsorbed ECM controls (FIG. 11D). Furthermore, while PHH CYP1A2 activity slowly declined over time in adsorbed ECM controls (30-35% of day 9 levels after 23 days), CYP1A2 activity on the nanofibers increased ≈1.4- to 2.4-fold between 9 and 17 days and then declined approximately 60-80% between days 17 and 23 of culture (FIG. 11D).

Maximal PHH CYP2A6 activities were 6-fold higher on the collagen nanofibers, 3.2-fold higher on the collagen/chitosan nanofibers, 6.6-fold higher on the collagen/PLECM nanofibers, 8.7-fold higher on the PLECM nanofibers, and 3.6-fold higher on the PLECM/chitosan nanofibers as compared to the adsorbed ECM controls (FIG. 11E). Furthermore, PHHs on the collagen/PLECM and PLECM/chitosan nanofibers displayed the highest CYP2A6 activity after 7 days; PHHs on the collagen nanofibers displayed the highest CYP2A6 activity after 15 days; PHHs on the collagen/chitosan and PLECM nanofibers displayed the highest CYP2A6 activity after 21 days (FIG. 11E).

Maximal PHH CYP2C9 activities were 3.4-fold higher on the collagen nanofibers, 3.5-fold higher on the collagen/PLECM nanofibers, 2.4-fold higher on the PLECM nanofibers, and 3.2-fold higher on the PLECM/chitosan nanofibers as compared to the adsorbed ECM controls; in contrast, while maximal PHH CYP2C9 activity on the collagen/chitosan nanofibers (day 9) was approximately 7% lower than the adsorbed ECM controls, it was 1.5- to 2.5-fold higher between 17 and 23 days of culture (FIG. 11F). Furthermore, while PHH CYP2C9 activities declined between 38% and 80% across all the cultures between 9 and 17 days of culture, CYP2C9 activities after 23 days of culture were still 2.7-fold higher on the collagen nanofibers, 1.5-fold higher on the collagen/chitosan nanofibers, 2.3-fold higher on the collagen/PLECM nanofibers, 3.5-fold higher on the PLECM nanofibers, and 1.4-fold higher on the PLECM/chitosan nanofibers as compared to the adsorbed ECM controls (FIG. 11F).

Maximal PHH CYP3A4 activities were 3.6-fold higher on the collagen nanofibers, 1.1-fold higher on the collagen/chitosan nanofibers, 4.4-fold higher on the collagen/PLECM nanofibers, 3.6-fold higher on the PLECM nanofibers, and 3.2-fold higher on the PLECM/chitosan nanofibers as compared to the adsorbed ECM controls (FIG. 11G). Furthermore, CYP3A4 activities increased 3- to 5.5-fold between days 7 and 15 for all the cultures, but then declined approximately 12-36% by day 21 (FIG. 11G). Statistical comparisons for nanofibers of FIGS. 11C-11G are shown in Table 5.

The results above showed that while nanofibers can upregulate the activities of several different CYP isoforms as compared to adsorbed ECM substrates, they were unable to stabilize CYP levels for several weeks, which suggests that ECM manipulations alone were necessary but not sufficient to maintain CYP activities in PHHs over prolonged culture; similar with other substrates in which ECM was the only microenvironmental manipulation for PHH monocultures in vitro.

TABLE 5
Albumin in PHH monocultures: p-value from 2-way ANOVA, using Dunnett's
multiple comparison test.

| Nanofiber | control | Day 7 | Day 11 | Day 15 | Day 19 | Day 23 |
|---|---|---|---|---|---|---|
| Collagen/PLECM | collagen | 0.012 | 0.0011 | <0.0001 | 0.0018 | ns |
| | PLECM | 0.0115 | 0.002 | <0.0001 | 0.0026 | ns |
| PLECM | collagen | ns | 0.049 | 0.0042 | 0.0015 | ns |
| | PLECM | ns | ns | 0.006 | 0.0021 | ns |
| PLECM/chitosan | collagen | ns | 0.0228 | ns | ns | ns |
| | PLECM | ns | 0.0379 | ns | ns | ns |

Urea in PHH monocultures: p-value from 2-way ANOVA, using Dunnett's multiple
comparison test.

| Nanofiber | control | Day 3 | Day 5 | Day 7 | Day 9 | Day 11 | Day 13 | Day 19 | Day 21 |
|---|---|---|---|---|---|---|---|---|---|
| Collagen/chitosan | collagen | 0.0188 | ns | ns | ns | ns | ns | ns | ns |
| | PLECM | ns | ns | ns | ns | ns | ns | ns | ns |
| Collagen/PLECM | collagen | 0.0094 | 0.0001 | 0.0003 | 0.0012 | 0.0017 | 0.0059 | 0.0009 | 0.0233 |
| | PLECM | ns | 0.0003 | 0.0008 | 0.0002 | 0.0117 | 0.008 | 0.0009 | 0.0214 |
| PLECM | collagen | ns | 0.0056 | 0.0457 | ns | ns | ns | 0.0006 | 0.0053 |
| | PLECM | ns | 0.0096 | ns | 0.0224 | ns | ns | 0.0006 | 0.0048 |
| PLECM/chitosan | collagen | ns | 0.0034 | ns | ns | 0.0338 | ns | 0.0324 | ns |
| | PLECM | ns | 0.0059 | ns | 0.0354 | 0.0338 | ns | 0.0314 | ns |

*ns: not significant

### Example 5: Effects of PLECM Microfibers on the Phenotype of PHH Monocultures

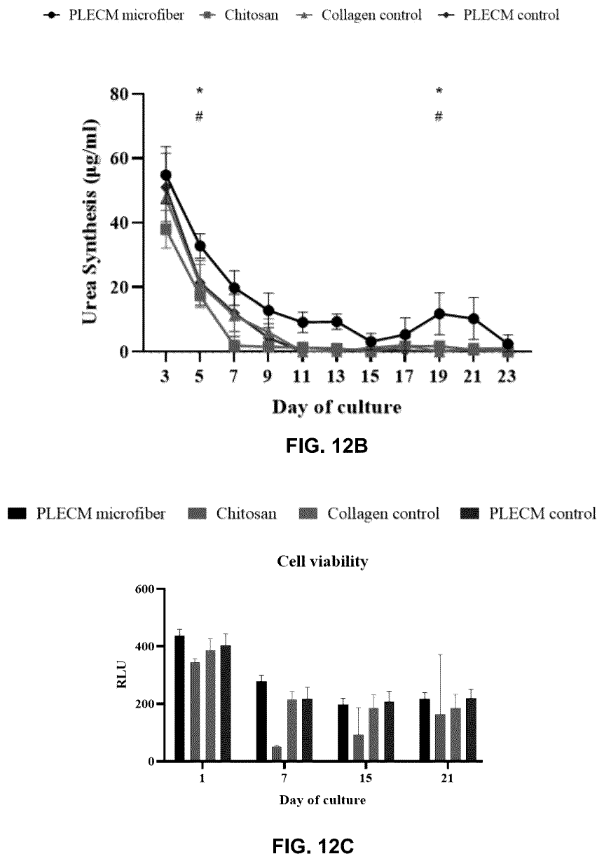

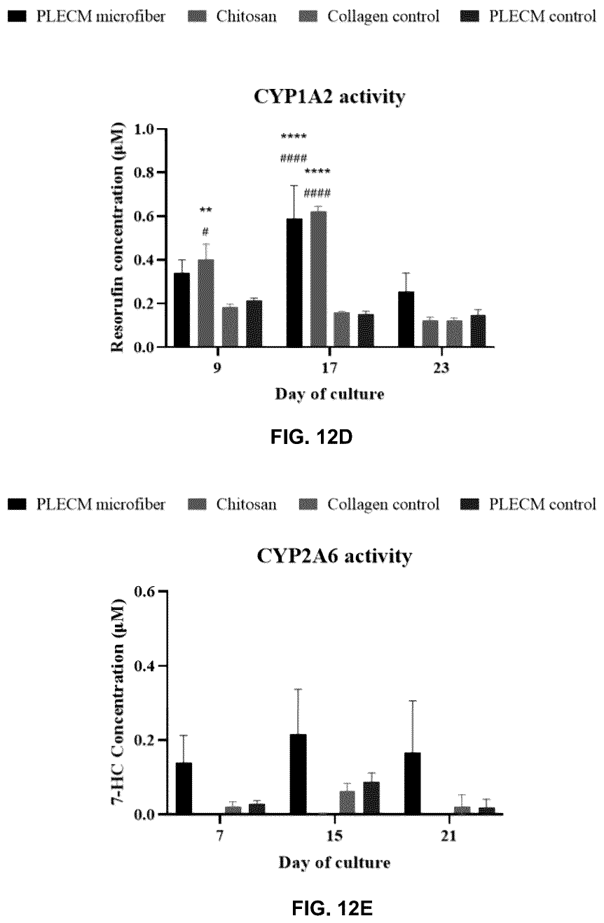

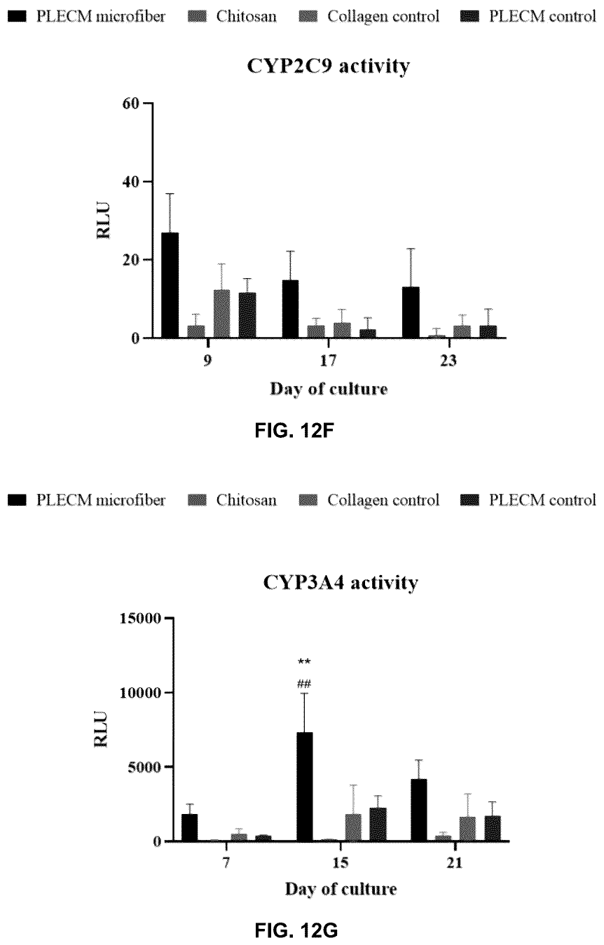

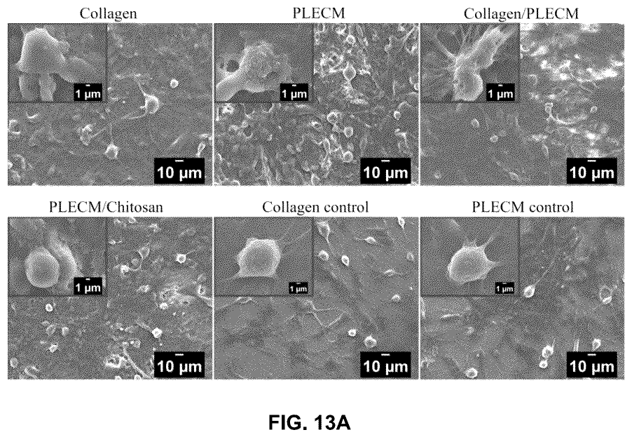

As on the nanofibers, PHHs on the 5% PLECM-only microfibers a) attached and infiltrated (FIG. 8), b) displayed similar viability overtime as the adsorbed ECM controls (FIG. 12A and FIG. 12C) displayed similar kinetics of albumin secretion (FIG. 12B), urea synthesis (FIG. 12C), and CYP enzyme activities (FIG. 12D-12G). Maximal albumin secretion on the PLECM-only microfibers was 4.3-fold higher, urea was 2.6-fold higher, CYP1A2 activity was 3-fold higher, CYP2A6 activity was 2.5-fold higher, CYP2C9 activity was 2.3-fold higher, and CYP3A4 activity was 3.2-fold higher than adsorbed ECM controls (FIG. 12A-12G). These results suggested that both nano- and micro-fibers allow for the upregulation of PHH functions as compared to adsorbed ECM substrates, likely due to the higher ECM surface area for PHHs to interact with on the fibrous matrices.

In contrast to PLECM-only microfibers, chitosan-only nanofibers caused a 60% reduction in the maximal PHH albumin secretion, a 73% reduction in CYP2C9 activity, and an 80% reduction in CYP3A4 activity as compared to the adsorbed ECM controls, while urea and CYP2A6 activity in chitosan-only nanofibers were undetectable after 3 weeks; however, maximal CYP1A2 activity on chitosan-only nanofibers was 3-fold higher than on the adsorbed ECM controls (FIG. 12A-12G). These results suggest that while the fibrous topography of the chitosan-only nanofibers can induce some PHH functions (e.g., CYP1A2) as compared to adsorbed ECM substrates, it is not sufficient to induce other PHH functions, likely due to the lack of adequate adhesion sites and biochemical signaling afforded for by fibers generated using proteins present in the liver (i.e., collagen I and whole liver ECM).

### Example 6: Effects of ECM Nanofibers on the Morphology and Secretory Functions of Cocultures Containing PHHs and 3T3-J2 Fibroblasts

Since PHH monocultures displayed continuously declining urea synthesis and CYP2C9 activity even on the ECM nanofibers as discussed above, cocultures of PHHs with 3T3-J2 fibroblasts were established, which were previously shown to improve the abovementioned functions on collagen-coated polystyrene, toward determining if ECM nanofibers could stabilize and further improve PHH functions in cocultures over the adsorbed ECM controls. Cocultures were executed on adsorbed ECM (collagen or PLECM) controls and selected collagen, PLECM, collagen/PLECM, and PLECM/chitosan nanofibers, since they showed some of the highest functions for PHH monocultures as discussed above.

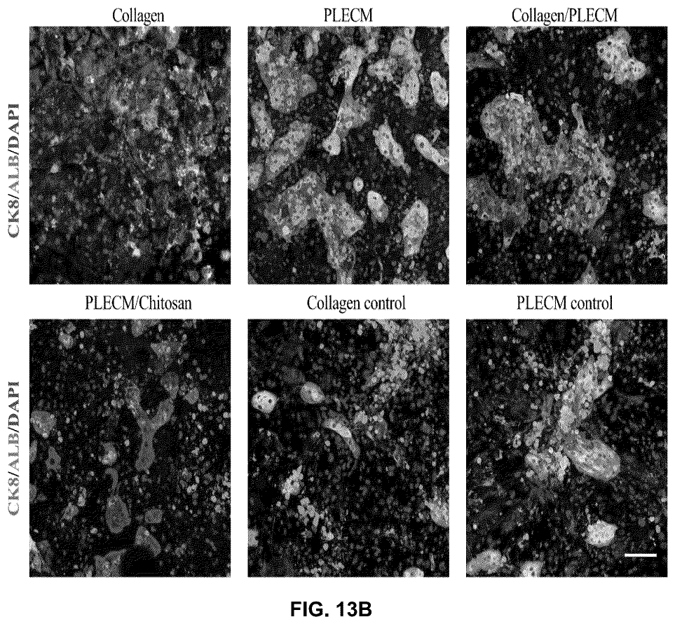

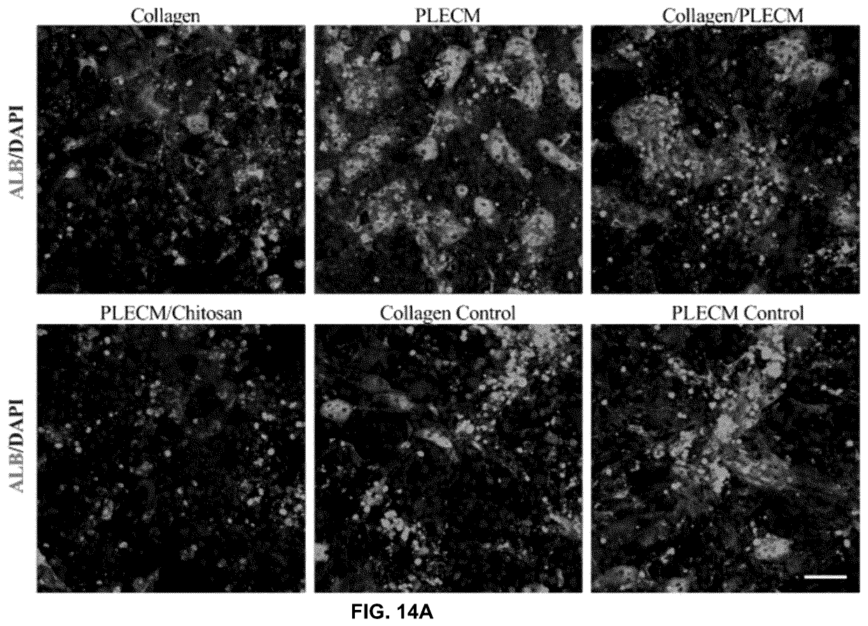

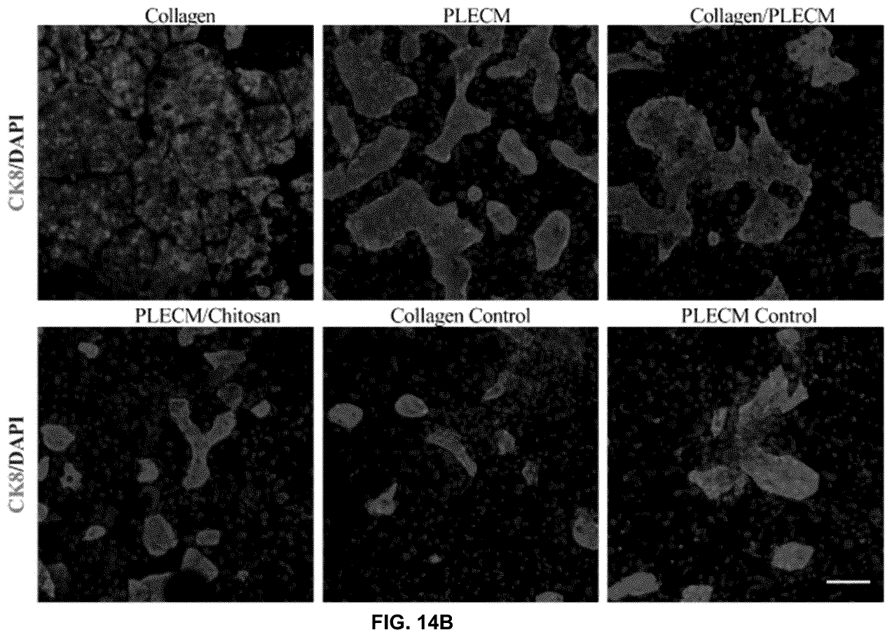

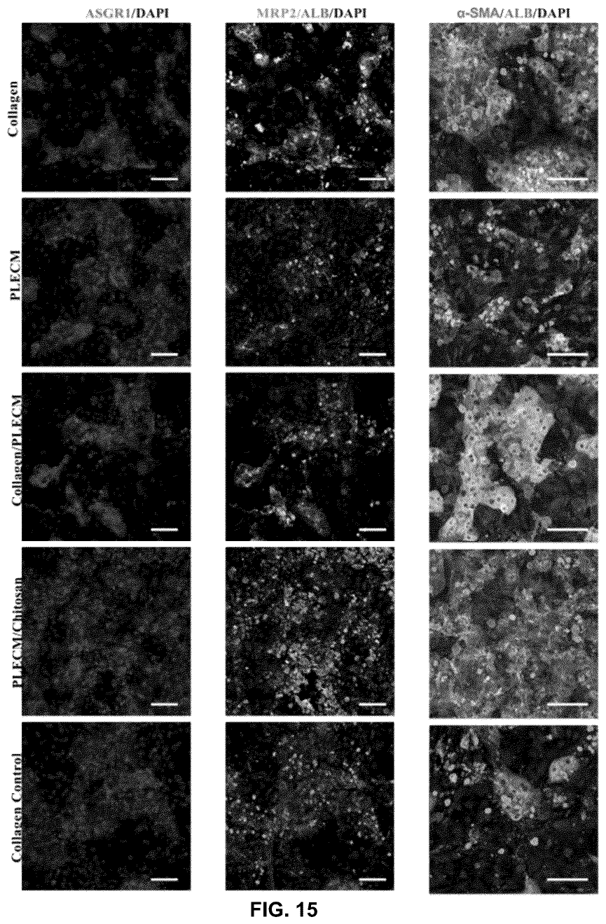

Upon cocultivation with 3T3-J2 fibroblasts, PHHs maintained similar spheroidal morphology for 23 days on the nanofibers containing collagen and PLECM and adsorbed ECM control substrates as assessed via SEM, albeit fewer PHHs were observed on the adsorbed ECM controls; interactions between the two cell types on the nanofibers were observed in high magnification SEM images (FIG. 13A). Coimmunostaining for human albumin and human CK8 showed that PHHs formed colonies around the mouse fibroblasts across all tested culture formats; however, in contrast to the adsorbed ECM controls, a greater number and larger PHH colonies were observed on the nanofibers, especially collagen, PLECM, and collagen/PLECM nanofibers (FIG. 13B). Additionally, PHHs displayed both albumin and CK8 staining on most of the nanofibers and particularly on the PLECM and collagen/PLECM nanofibers, (FIG. 13B; and FIG. 14A-14B). Finally, ASGR1 and MRP2 (major transporter present on the apical/canaliculi surface of hepatocytes) were also detected on PHHs cultured on nanofibers and adsorbed controls, albeit MRP2 staining patterns were dependent on PHH cluster size with larger clusters containing a greater number of MRP-positive bile canaliculi between adjacent PHHs; the fibroblasts were positive for α-SMA (FIG. 15). These results showed that PHHs maintain several major differentiated phenotypic markers (albumin, CK8, ASGR1, MRP2) when cocultured with the fibroblasts on the nanofibers, albeit the expression of these markers is dependent on the colony size, which was expected since local homotypic interactions between PHHs are known to influence their differentiated state.

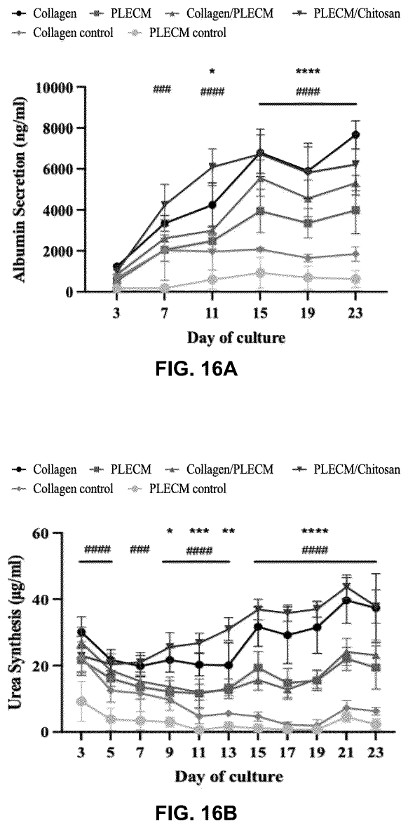

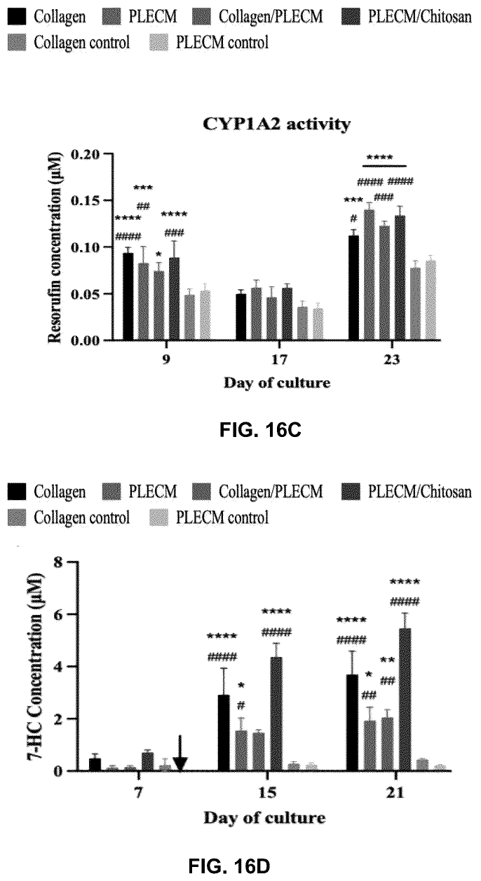

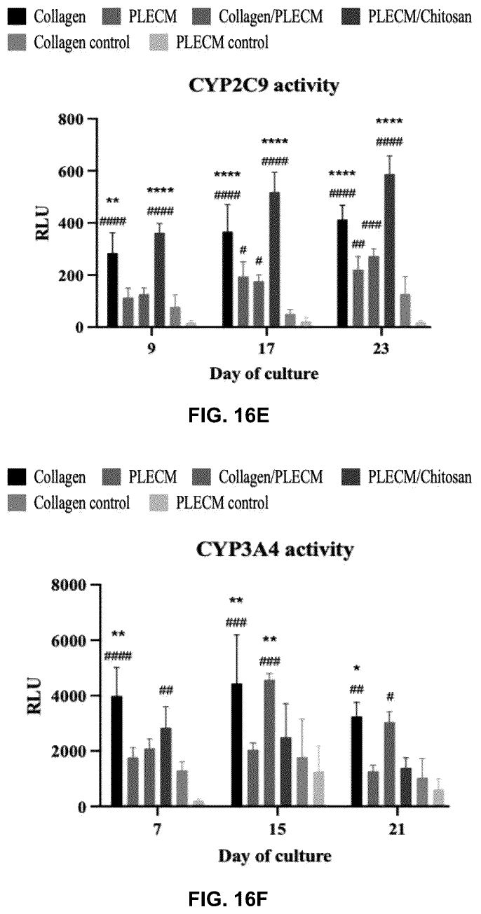

Cocultures on the adsorbed ECM controls displayed peak albumin secretion by day 15, which then declined by 11% to 32% between 15 and 23 days of culture. In contrast, albumin secretion on the nanofibers peaked by day 15 and remained relatively steady for 23 days (FIG. 16A). Interestingly, cocultures showed 2.2-fold higher albumin secretion on the adsorbed collagen control as compared to the adsorbed PLECM control. Furthermore, the maximal albumin secretion rates were 3.7-fold higher on the collagen nanofibers, 1.9-fold higher on the PLECM nanofibers, 2.7-fold higher on the collagen/PLECM nanofibers, and 3.2-fold higher on the PLECM/chitosan nanofibers as compared to the adsorbed collagen control (FIG. 16A).

Coculture urea synthesis declined by 57-67% of day 3 levels after 9 days in culture on the adsorbed ECM controls, though cocultures synthesized 2- to 3-fold higher urea on adsorbed collagen as compared to adsorbed PLECM. In contrast, cocultures on the nanofibers showed either relatively stable (PLECM and collagen/PLECM) or increasing urea synthesis over time (PLECM/chitosan up to 1.9-fold and collagen up to 1.3-fold relative to day 3 levels) (FIG. 16B). Furthermore, the urea synthesis rates were 5.9-fold higher on the collagen nanofibers, 3-fold higher on the PLECM nanofibers, 3.7-fold higher on the collagen/PLECM nanofibers, and 6-fold higher on the PLECM/chitosan nanofibers as compared to the adsorbed collagen control after 23 days (FIG. 16B).

The results showed that in contrast to PHH monocultures, coculture with fibroblasts upregulated and stabilized both PHH albumin and urea secretions, suggesting that the synergy between the nanofiber composition/topography and signaling from the fibroblasts are important for such outcomes.

### Example 7: Effects of ECM Nanofibers on the CYP Enzyme Activities of Cocultures Containing PHHs and 3T3-J2 Fibroblasts

CYP1A2 activities in all the cocultures decreased between 9 and 17 days and then increased by day 23 to levels higher than levels on day 9; furthermore, maximal coculture CYP1A2 activities were 1.4-fold higher on the collagen nanofibers, 1.7-fold higher on the PLECM nanofibers, 1.5-fold higher on the collagen/PLECM nanofibers, and 1.6-fold higher on the PLECM/chitosan nanofibers as compared to the adsorbed collagen control (FIG. 16C).

In contrast to CYP1A2, CYP2A6 activities in all the cocultures increased over time; additionally, maximal coculture CYP2A6 activities were 8.4-fold higher on the collagen nanofibers, 4.4-fold higher on the PLECM nanofibers, 4.7-fold higher on the collagen/PLECM nanofibers, and 12.5-fold higher on the PLECM/chitosan nanofibers as compared to the adsorbed collagen control, which itself had 2-fold higher maximal CYP2A6 activity than the adsorbed PLECM control (FIG. 16D).

As with CYP2A6, CYP2C9 activities in all cocultures increased over time; additionally, maximal coculture CYP2C9 activities were 3.2-fold higher on the collagen nanofibers, 1.7-fold higher on the PLECM nanofibers, 2.1-fold higher on the collagen/PLECM nanofibers, and 4.6-fold higher on the PLECM/chitosan nanofibers as compared to the adsorbed collagen control, which itself had 6.5-fold higher maximal CYP2C9 activity than the adsorbed PLECM control (FIG. 16E).

Finally, CYP3A4 activities peaked after 15 days across all the cocultures except the PLECM/chitosan nanofibers that displayed highest activity after 7 days, and then declined between 27% and 52% from the peak activity by 21 days; furthermore, maximal coculture CYP3A4 activities were 2.5-fold higher on the collagen nanofibers, 1.1-fold higher on the PLECM nanofibers, 2.6-fold higher on the collagen/PLECM nanofibers, and 1.6-fold higher on the PLECM/chitosan nanofibers as compared to the adsorbed collagen control, which itself had 1.4-fold higher maximal CYP3A4 activity than the adsorbed PLECM control (FIG. 16F). Statistical comparisons for nanofibers of FIGS. 16C-16F are shown in Table 6.

These results showed that while the synergy between nanofiber composition/topography and fibroblast coculture could upregulate both abundant (CYP1A2, 2C9, 3A4) and less abundant (CYP2A6) CYPs over prolonged culture as compared to adsorbed ECM substrates, such a synergy was able to better stabilize some CYP isoforms (CYP2A6, 209) than others (CYP1A2, 3A4) over approximately 3 weeks of culture.

TABLE 6
Albumin in PHH/3T3-J2 co-cultures: p-value from 2-way ANOVA with Dunnett's
multiple comparison test.

| Nanofiber | control | Day 7 | Day 11 | Day 15 | Day 19 | Day 23 |
|---|---|---|---|---|---|---|
| PLECM | collagen | ns | ns | ns | ns | 0.0255 |
| | PLECM | ns | ns | 0.0008 | 0.0035 | 0.0002 |
| Collagen/PLECM | collagen | ns | ns | <0.0001 | 0.0012 | <0.0001 |
| | PLECM | 0.0089 | 0.01 | <0.0001 | <0.0001 | <0.0001 |
| PLECM/chitosan | collagen | 0.0207 | <0.0001 | <0.0001 | <0.0001 | <0.0001 |
| | PLECM | <0.0001 | <0.0001 | <0.0001 | <0.0001 | <0.0001 |

Urea in PHH/3T3-J2 co-cultures: p-value from 2-way ANOVA with Dunnett's multiple comparison test.

| Nanofiber | control | Day 3 | Day 5 | Day 7 | Day 9 | Day 11 | Day 13 | Day 15 | Day 17 | Day 19 | Day 21 | Day 23 |
|---|---|---|---|---|---|---|---|---|---|---|---|---|
| PLECM | collagen | ns | ns | ns | ns | ns | ns | 0.0011 | 0.007 | 0.0028 | 0.001 | 0.0043 |
| | PLECM | 0.0075 | 0.0089 | 0.0423 | ns | 0.0223 | 0.0179 | <0.0001 | 0.002 | 0.001 | <0.0001 | 0.0001 |
| Collagen/ | collagen | ns | ns | ns | ns | ns | ns | 0.0223 | 0.0303 | 0.0019 | 0.0001 | 0.0001 |
| PLECM | PLECM | <0.0001 | 0.0011 | 0.0121 | 0.0314 | 0.0169 | 0.0244 | 0.0013 | 0.0101 | 0.0006 | <0.0001 | <0.0001 |
| PLECM/ | collagen | ns | ns | ns | 0.0004 | <0.0001 | <0.0001 | <0.0001 | <0.0001 | <0.0001 | <0.0001 | <0.0001 |
| chitosan | PLECM | 0.0025 | 0.0002 | <0.0001 | <0.0001 | <0.0001 | <0.0001 | <0.0001 | <0.0001 | <0.0001 | <0.0001 | <0.0001 |

*ns: not significant

### Example 8: Long-Term Functions of PHH/3T3-J2 Cocultures on the Collagen/PLECM Nanofibers

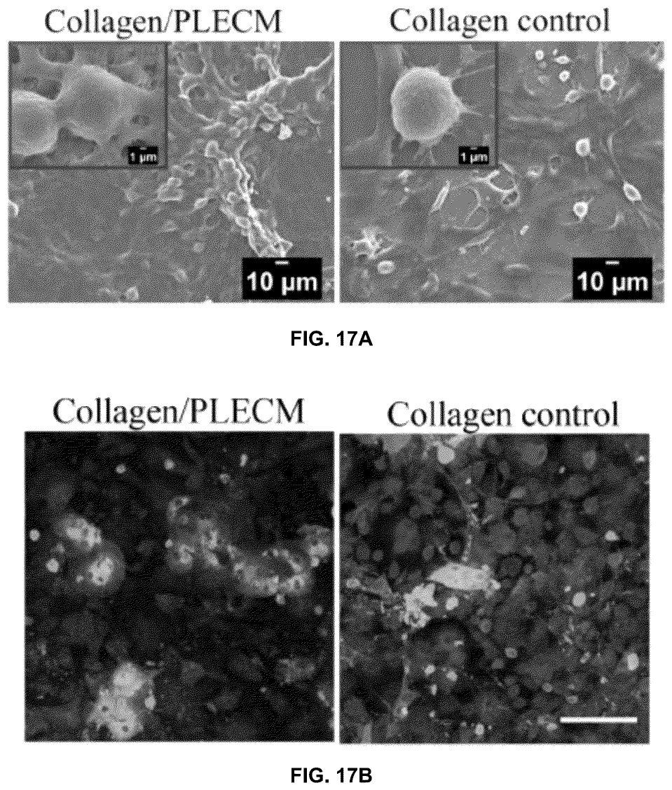

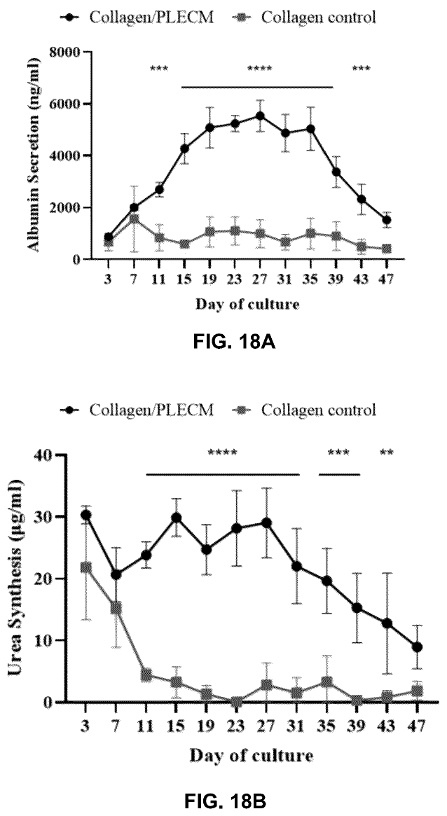

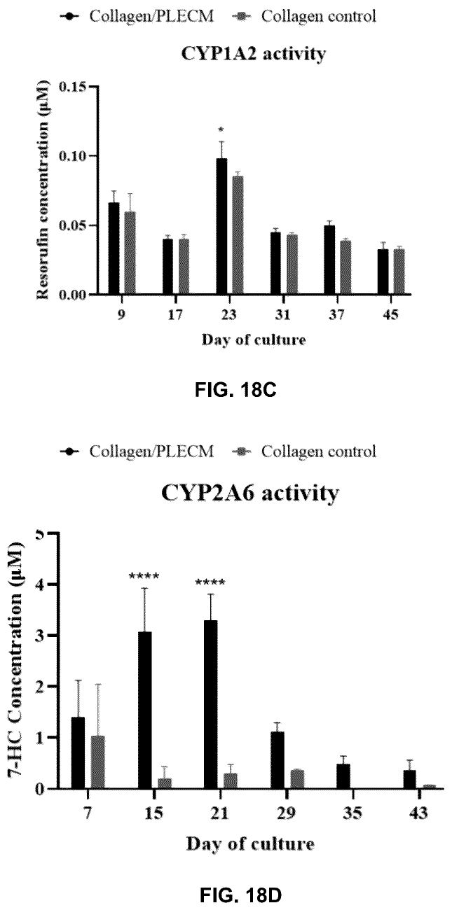

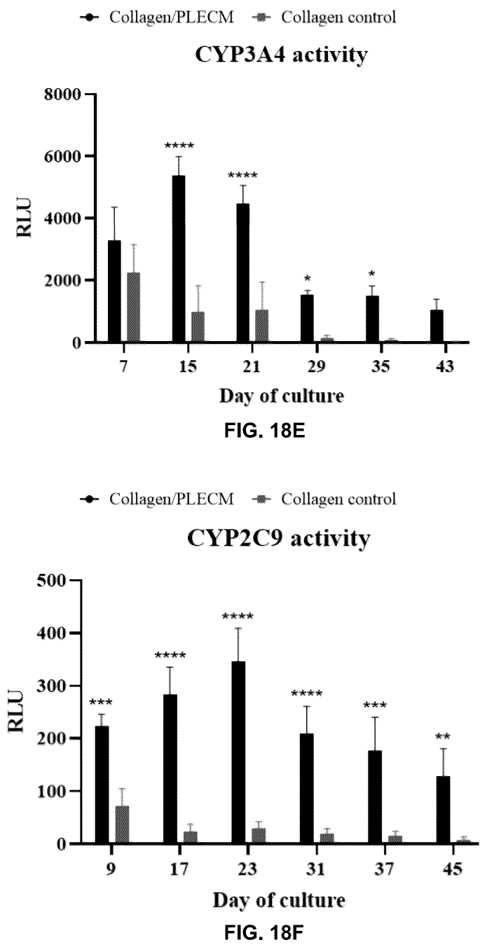

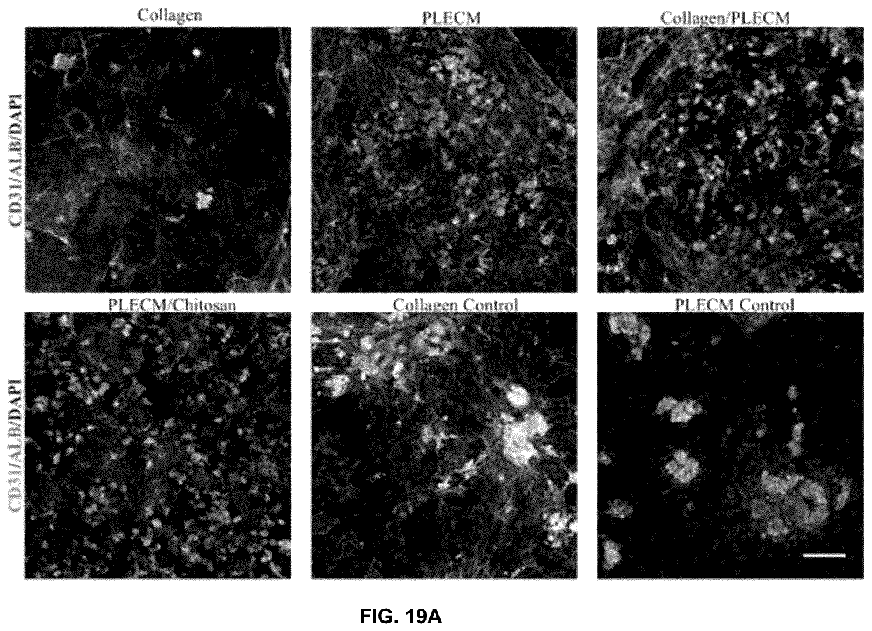

Next, PHH/3T3-J2 were cocultured for 47 days on the collagen/PLECM nanofibers that showed the highest CYP3A4 activity above, while using adsorbed collagen as a control. Even after 47 days, PHHs and 3T3-J2s were detected via SEM and immunostaining on the above substrates (FIG. 17A-17B). At the functional level, once cocultures on the nanofibers reached their peak albumin secretion rates over 15-19 days, the levels remained relatively stable for 35 days in culture, after which a decline was observed down to 27% of the peak levels by day 47; nonetheless, even after 47 days, albumin secretion rates in cocultures on the nanofibers were 3.6-fold higher than the adsorbed collagen control (FIG. 18A). Urea synthesis had similar trends as albumin above, with a 5-fold higher rate observed as compared to the adsorbed collagen control even after 47 days in culture (FIG. 18B). CYP1A2 activity was relatively stable in the cocultures for 45 days, while CYP2A6, 3A4, and 2C9 activities peaked by 3 weeks and then displayed variable decline rates over 47 days; however, even after 47 days, CYP2A6 was 5-fold higher, CYP3A4 was 34-fold higher, and CYP2C9 was 17-fold higher on the collagen/PLECM nanofibers as compared to the adsorbed collagen control (FIG. 18C-18F). These results showed that while the synergy between nanofiber composition/topography and fibroblast coculture could upregulate several major functions of the liver for approximately 7 weeks of culture as compared to adsorbed ECM controls, the optimal window of functional stability (for drug screening and other biological inquiries) was approximately 3 weeks.

### Example 9: Effects of ECM Nanofibers on the Phenotype of Cocultures Containing PHHs and Human LSECs

Since the 3T3-J2 fibroblasts are of mouse origin, their coculture with PHHs is useful for drug screening application, but not for applications in regenerative medicine (i.e., cell-based therapy). In contrast, LSECs are present in the liver and previous studies with rat hepatocytes have shown that these cells can stabilize some hepatic functions on adsorbed ECM substrates. However, it has been shown that human LSECs were unable to stabilize PHH functions on adsorbed ECM substrates without the inclusion of the 3T3-J2 fibroblasts.

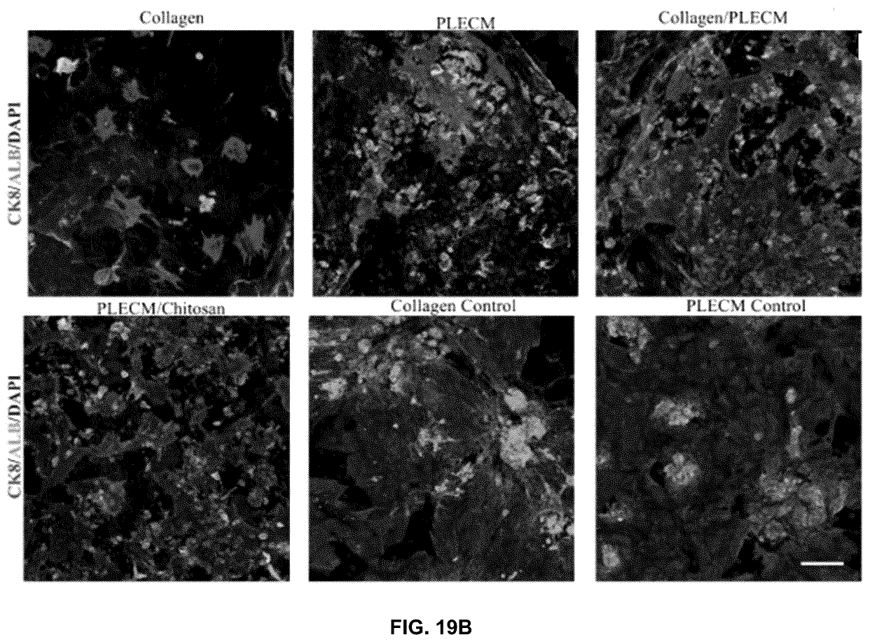

Here, it was hypothesized that the synergy between nanofiber composition/topography and LSEC interactions could mitigate the above limitation with PHH/LSEC cocultures on adsorbed ECM substrates. Toward that end, PHH/LSEC cocultures were generated on the same nanofibers and adsorbed ECM control substrates as those used for PHH/3T3-J2 cocultures above. Immunostaining for CD31 and albumin showed the presence of endothelial cells and PH Hs across all culture formats, respectively, albeit significantly fewer endothelial cells and PHHs were observed on the PLECM adsorbed control (FIG. 19A-19B), which was unexpected given the physiological relevance of PLECM over collagen-I alone. Coimmunostaining for albumin and CK8 showed that many spread-out PHHs had detectable CK8 but not albumin, and PHHs on collagen nanofibers were less spread-out but fewer in numbers than the other conditions (FIG. 19A-19B), which suggested that some PHHs de-differentiate in the PHH/LSEC cocultures irrespective of substrate type.

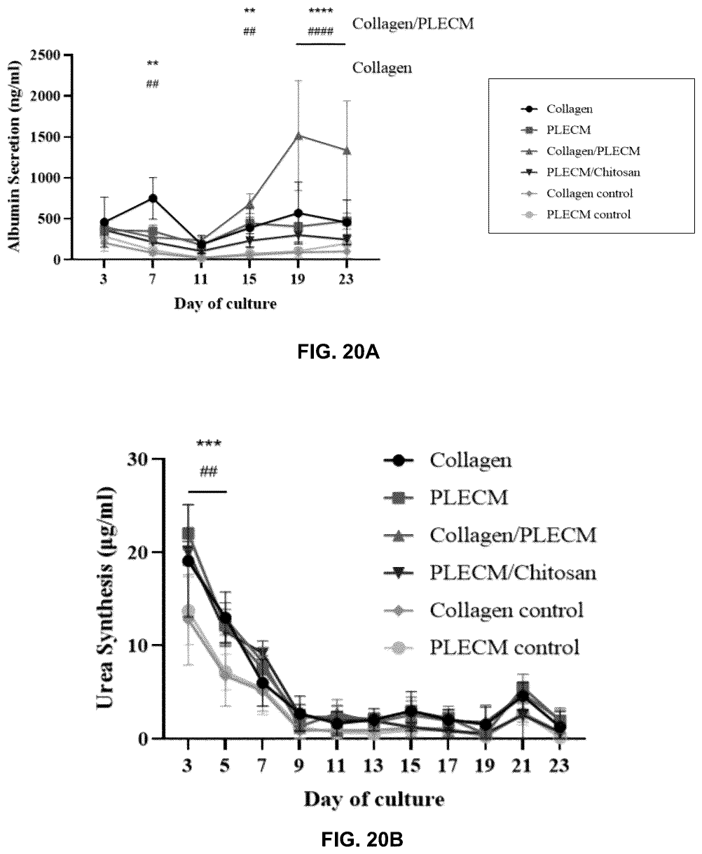

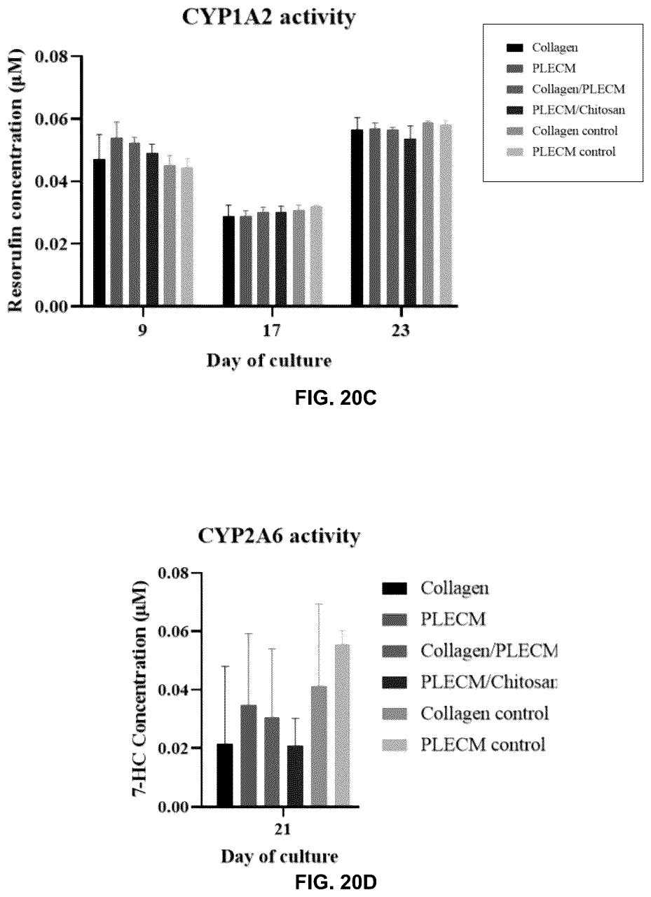

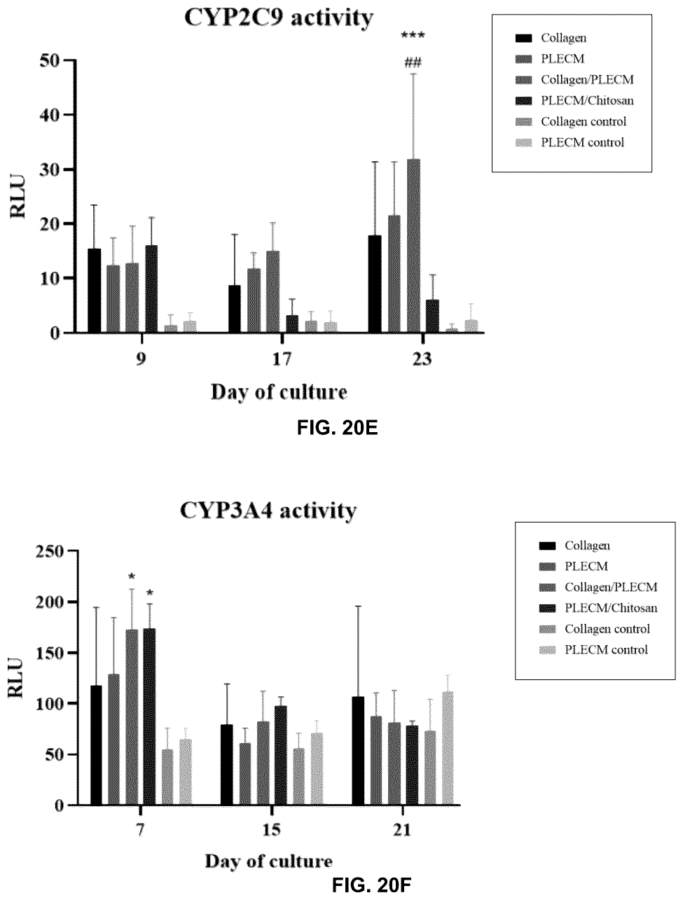

PHH/LSEC cocultures on the adsorbed ECM controls displayed precipitously declining albumin and urea secretions (FIG. 20A-20B), which is consistent with our previous findings on PHH/LSEC cocultures fabricated on polystyrene with adsorbed collagen I. In contrast, albumin secretion either remained relatively stable or increased over time in PHH/LSEC cocultures on the selected ECM nanofibers, though maximal secretions in cocultures on the nanofibers were 3.7 to 18.6-fold lower than the maximal secretions in corresponding PHH/3T3-J2 cocultures (FIG. 16A). However, urea synthesis in the PHH/LSEC cocultures displayed the same precipitous decline across all the substrates as that observed with PHH monocultures above. Finally, CYP activities in the PHH/LSEC cocultures were generally statistically similar over time on the nanofibers as compared to adsorbed ECM controls (FIG. 20C-20F). Table 7 shows statistical comparison of nanofibers of FIG. 20C-20F.

These results suggested that the major contributors to the upregulation and stabilization of albumin secretion were the nanofiber composition and topography as opposed to coculture with LSECs since the functional trends on nanofibers versus adsorbed ECM substrates were similar across PHH monocultures and PHH/LSEC cocultures, whereas they were significantly different (more differentiated over several weeks in vitro) in PHH/3T3-J2 cocultures, potentially due to key liver-like molecules secreted/presented by the fibroblasts.

TABLE 7
Urea in PHH/LSEC co-cultures; p-value from 2-way
ANOVA with Dunnett's multiple comparison test.

| Nanofiber | control | Day 3 | Day 5 |
|---|---|---|---|
| PLECM | collagen | <0.0001 | 0.005 |
| | PLECM | <0.0001 | 0.011 |
| Collagen/PLECM | collagen | 0.0003 | 0.001 |
| | PLECM | 0.0024 | 0.0025 |
| PLECM/chitosan | collagen | <0.0001 | 0.0168 |
| | PLECM | 0.0006 | 0.0338 |

### Summary of Examples

Experiments were undertaken to test if PHH monocultures and PHH/NPC cocultures would display significantly higher liver functions for several weeks on nanofibrous scaffolds fabricated using ECM proteins found in the liver. Toward testing this hypothesis, a method was developed to electrospin stable nanofibers of decellularized porcine liver ECM (PLECM) given the robust availability of healthy porcine livers and the ability of porcine liver ECM to support some rat hepatocyte functions in vitro. As a control ECM, rat tail collagen-I and the polysaccharide chitosan were utilized. Rat tail collagen-I is widely used for PHH culture due to its ready availability and cost-effectiveness. Chitosan has been previously shown to stabilize collagen scaffolds and support some rat hepatocyte functions in vitro. PHHs were cultured on their own and with well-established NPC types, namely 3T3-J2 murine embryonic fibroblasts and primary human liver sinusoidal endothelial cells (LSECs), on the different nanofibrous scaffolds and nonfibrous (i.e., adsorbed) control substrates; cell morphology, viability, immunostaining patterns, and phenotypic functions (albumin and urea secretions, and cytochrome-P450 (CYP) 1A2, 2A6, 2C9, and 3A4 enzyme activities) were assessed for up to 47 days in culture

Previous studies have utilized synthetic polymers alone or those blended with a concentrated liver ECM solution to generate nanofibers for liver culture. In contrast, as shown in the disclosure several steps of the decellularization process were modified to prepare dry PLECM for electrospinning without the need for synthetic polymers, including pepsin-based digestion of PLECM to break crosslinked sites on ECM and subsequently removing pepsin via dialysis and lyophilizing. This protocol should be applicable with few modifications to human LECM, though PLECM may still be most suited for human liver cell culture given the unpredictable conditions and quality of the transplant-rejected human livers.

Synthetic or hydrophobic macromolecules are typically used for generating nanofibers for cell culture since they are stable in polar cell culture medium, easy to handle, and do not require chemical crosslinking. However, tissue-derived ECM can better support the functions of rodent hepatocytes and transformed hepatic cell lines, and thus it is desirable to generate nanofibers from such ECM for PHH culture. Here, the difficulties associated with using hydrophilic ECM biomolecules were overcome by a) electrospinning directly onto glass coverslips to enable microscopic imaging of the cells via transmitted light microscopy and b) chemically crosslinking the nanofibers using EDC/NHS in 90% ethanol (glutaraldehyde vapor for chitosan-only due to swelling in ethanol) to allow for easier handling of the nanofibers in culture. PLECM at 2% w/v could be used in combinations with other ECM molecules to generate nanofibers but using PLECM at 5% w/v led to the formation of microfibers, the mechanisms of which are not clear but could be related to components within PLECM adhering more strongly to each other at higher concentrations during electrospinning. Furthermore, increasing the solution concentration for electrospinning often leads to higher diameter fibers; nonetheless, all nanofibers maintained statistically similar diameter ranges before and after crosslinking (approximately 200 nm), which allowed us to test the effects of nanofiber composition on hepatic functions as opposed to wide variations in diameter.

Nanofibers fabricated using collagen I, PLECM, and their blends maintained fiber morphology and average porosities (approximately 46-60%) following crosslinking; however, the average pore areas decreased for some of the nanofibers following crosslinking, likely due to a high degree of swelling in the 90% ethanol-containing crosslinking solution. Nonetheless, cells were still able to interact with and penetrate into several of the crosslinked nanofibers. Furthermore, collagen/PLECM (approximately 0.1 MPa Young's modulus), PLECM (approximately 0.3 MPa), collagen/chitosan (approximately 0.3 MPa), and PLECM/chitosan (approximately 0.4 MPa) nanofibers were softer than the collagen-only and chitosan-only nanofibers (approximately 0.8-1.4 MPa), potentially due to noncollagen molecules present in PLECM and chitosan interfering with the tight crosslinking/packing between collagen-I molecules. While the stiffness of the nanofibers was higher than that of native liver (approximately 0.5-4 kPa), it was three orders of magnitude lower than plastic or glass (GPa). Finally, comparison of the FTIR and XPS spectra of the fibers and the individual macromolecules showed similar polarizable functional groups and elemental compositions, respectively, suggesting that the electrospinning process did not significantly change the biochemistry of the ECM.

When incubated in cell culture medium, pure collagen, pure PLECM, and collagen/chitosan nanofibers lost some porosity and changed morphology over time as compared to the collagen/PLECM and PLECM/chitosan blended fibers that maintained porosity and morphology even after 23 days. It is possible that unlike pure collagen I, PLECM has other proteins (e.g., collagen IV) that act to provide mechanical stability to the collagen or chitosan fibrillar ECM scaffold, and the higher total concentrations of macromolecules in the collagen/PLECM and chitosan/PLECM blends used for electrospinning generated additional sites for interaction and crosslinking than PLECM alone. Regardless of the variable morphological stability of different nanofiber compositions in cell culture medium over several weeks, they were suitable for liver cell culture and phenotypic assessments.

SEM showed that PHHs maintained similar morphology for several weeks on nanofibers containing collagen and PLECM, whereas PHH numbers were considerably lower on the adsorbed ECM controls following prolonged culture. In contrast, chitosan-only nanofibers resulted in lower PHH attachment which may be due to the lack of collagens typically needed for robust PHH attachment. Immunostaining showed that PHHs on nanofibers were positive for intermediate filament, CK8; however, co-expression of more differentiated/specialized markers, albumin and ASGR1, was variable across PHHs on nanofibers, suggesting some level of PHH de-differentiation in monocultures even on nanofibers.

After a 20-40% drop in PHH viability over 7 days, values were relatively stable until 23 days; such a drop in viability is likely due to PHH death and detachment, as is common with other culture models, given that not all viable PHHs that attach to a substrate will survive in vitro due to the variable health of cells isolated from human livers. Despite this loss of some viability in the first week, nanofibers caused to up to approximately 5-fold higher maximal albumin secretion, ≈3-fold higher CYP1A2 activity, ≈9-fold higher CYP2A6 activity, approximately 3.5-fold higher CYP2C9 activity, and ≈4-fold higher CYP3A4 activity than adsorbed ECM. PLECM-only nanofibers caused the highest maximal CYP2C9 activity while collagen/PLECM nanofibers caused the highest maximal levels for other functions (albumin, urea, and CYP1A2/2A6/3A4 activities). Collagen-only nanofibers generally ranked second or third in the induction of maximal functions, while PLECM/chitosan and collagen/chitosan blended nanofibers ranked lowest in their induction of PHH functions. Even PLECM microfibers (approximately 1 μm) caused similar induction of functions in PHH monocultures as nanofibers, which suggests that the ability of cells to interact with a larger surface area of fibers in the approximately 200-1000 nm range is beneficial for the induction of PHH functions over adsorbed ECM.

While others have shown the independent effects of ECM stiffness and porosity on hepatocyte functions, our study is the first to evaluate the synergistic effects of ECM stiffness, topography, porosity, and available surface area for cell attachment/infiltration on the long-term functions of PHHs. The nanofibers were found to be significantly softer (approximately 0.1-1.4 MPa) than glass/plastic and lower stiffness (protein hydrogels) has been previously shown to maintain the differentiated functions of mouse hepatocytes via enhanced expression of master transcription factor, hepatocyte nuclear factor 4 alpha (HNF4alpha). HNF4alpha expression is inhibited by stiffer surfaces through the induction of the Rho/Rho-associated protein kinase pathway; whether such a mechanism partly underlies the functional upregulation in PHHs on our nanofiber-based platform remains to be elucidated. Another variable that likely is important for the functional upregulation in PH Hs on nanofibers is porosity since previously collagen foams with pore size between 10 and 80 μm caused 50-fold higher albumin secretion in hepatocytes than nonporous gels, presumably due to better transport of key signaling molecules throughout the culture in the porous constructs. However, neither porosity nor topography were sufficient to induce the highest or most stable PHH functions here as shown with chitosan-only nanofibers relative to the liver ECM-based nanofibers. Such an outcome was not entirely unexpected since PHHs are adherent cell types that function better on collagenous substrates as opposed to substrates containing noncollagenous proteins, likely due to optimal focal adhesion formation and ensuing formation of a cortical actin network near the cell membranes (vs actin stress fiber formation) that helps stabilize the bile canaliculi toward polarizing the hepatocyte. Finally, since PLECM-only and collagen/PLECM nanofibers induced the highest PHH functions than collagen-only and chitosan-blended collagen or PLECM nanofibers, we hypothesize that the optimal combinations and concentrations of liver ECM proteins (e.g., collagens Ill and IV, fibronectin, laminin) in PLECM were responsible for such outcomes, whereas presence of chitosan inhibited PHH interactions with their native ECM proteins; indeed, in previous studies using recombinant liver inspired ECM proteins spotted onto microarray substrates, it was shown that specific ECM combinations can unexpectedly either induce or inhibit PHH functions, likely via complex and yet undiscovered mechanotransduction mechanisms that target hepatic transcription factors such as HNF4alpha.

Urea and CYP2C9 activity showed a precipitous decline in PHH monocultures on nanofibers, which is consistent with previous findings that ECM manipulations alone are necessary but not sufficient to stabilize diverse hepatic functions. In contrast, 3T3-J2 fibroblasts were previously shown to induce and stabilize hepatic functions, including CYP2C9 activity and urea synthesis, for several weeks on adsorbed ECM, though levels need further enhancement to be closer to physiological levels. Thus, PHH/3T3-J2 cocultures were generated to determine if an in vivo-like fibrous ECM scaffold could further induce hepatic functions than the adsorbed ECM.

ECM nanofibers coupled with fibroblasts led to a 3D/spheroidal PHH morphology for 23 days. Most PHHs in the cocultures coexpressed CK8, albumin, ASGR1, and MRP2, suggesting a well-differentiated and polarized phenotype. Additionally, PHH/fibroblast cocultures on nanofibers showed up to 3.7-fold higher maximal albumin secretion, 6-fold higher urea synthesis, 1.7-fold higher CYP1A2 activity, 12.5-fold higher CYP2A6 activity, 4.6-fold higher CYP2C9 activity, and 2.6-fold higher CYP3A4 activity than on adsorbed ECM. Both albumin and urea secretions in cocultures on the nanofibers either increased or remained relatively stable once steady state was reached, whereas secretions declined in cocultures on the adsorbed ECM, albeit not in the same precipitous way as with PHH monocultures. CYP2C9, 1A2, and 2A6 activities showed similar trends as albumin and urea secretions, whereas CYP3A4 activity showed a 27% decline from its peak value by day 23.

Functional assessment of PHH/3T3-J2 cocultures on the collagen/PLECM nanofibers for 47 days showed that the relative stability of albumin/urea secretions and CYP activities was maintained for approximately 5 and approximately 3 weeks, respectively, except for CYP1A2 activity that displayed stability for the full 47 days. Nonetheless, even after 47 days, albumin/urea secretions and CYP2A6 activity were 3.6- to 5-fold higher, while CYP3A4 and CYP2C9 activities were 17- to 34-fold higher in cocultures on the nanofibers as compared to the adsorbed ECM. Finally, the presence of the fibroblasts appeared to stabilize nanofiber morphology for long-term cultures, potentially due to additional ECM secreted by the fibroblasts to mechanically stabilize the fibrous scaffold.

It has been previously showed that the 3T3-J2 fibroblasts induce the highest level of functions in PH Hs on adsorbed ECM relative to primary LSECs, Kupffer cells, and hepatic stellate cells, which may be due to the de-differentiation of these liver NPCs that also occurs alongside that of PHHs in vitro. In contrast, the embryonic 3T3-J2 fibroblasts could potentially provide developmentally appropriate differentiation cues to prevent PHH de-differentiation in vitro; indeed, 3T3-J2 fibroblasts express molecules present in the liver, including truncated-cadherin and decorin, though the multifactorial mechanism by which these fibroblasts induce the functions of hepatocytes from multiple species remains unelucidated. Nonetheless, here it was hypothesized that on a more in vivo-like ECM substrate, LSECs could induce PHH functions to a higher degree than on adsorbed ECM and potentially at higher levels than the murine fibroblasts. We found that, in contrast to a precipitous decline on adsorbed ECM controls, albumin secretion remained relatively stable or increased over time in PHH/LSEC cocultures on PLECM and/or collagen nanofibers, though levels were still lower than in the PHH/3T3-J2 cocultures. Other measured functions (urea and CYP activities) were statistically similar across PHH/LSEC cocultures on the nanofibers and adsorbed ECM, suggesting that LSEC-mediated support of PHHs may necessitate additional microenvironmental cues, such as further culture medium optimizations and/or coculture with other liver NPCs, to be fully physiologically-relevant and optimal. Finally, while passaged LSECs were used here to obtain sufficient numbers for coculture with PHHs, freshly isolated human LSECs that better retain their in vivo-like phenotype (e.g., fenestrae) may induce higher levels of PHH functions, albeit such cells are logistically and economically difficult to implement for routine experimentation.

Even though of murine origin, 3T3-J2 fibroblasts have been previously shown extensively to not interfere with the use of stabilized PHHs in cocultures for drug metabolism and toxicity screening and to model key features of global diseases, such as hepatitis B viral infection, malaria infection, and nonalcoholic fatty liver disease. Thus, the functionally enhanced PHH/3T3-J2 cocultures on ECM nanofibers disclosed here could be utilized for similar in vitro applications, but with a more in vivo-like ECM scaffold and higher functions than on adsorbed ECM, while cocultures containing only human and liver cells will be best suited for use in regenerative medicine.

In summary, a novel protocol to electrospin PLECM and collagen I into nanofibers was developed without the need for synthetic polymer blends. These natural ECM nanofibers were then utilized to show that PHH functions were significantly enhanced for several weeks on the ECM nanofibers relative to the conventionally adsorbed ECM controls, and that PHH/3T3-J2 cocultures displayed higher and stable functions for 5 weeks on the nanofibers than on the adsorbed ECM controls. The disclosed platform can be optimized and utilized for drug screening, disease modeling, and regenerative medicine, as well as to investigate the molecular mechanisms underlying the effects of ECM composition and topography on human liver functions.

What is claimed is:

1. A fiber comprising: an electrospun decellularized liver extracellular matrix (ECM).

2. The fiber of claim 1, wherein the liver ECM is a porcine liver ECM or human liver ECM.

3. The fiber of claim 2, wherein the fiber is a microfiber with an average diameter of about 1000 nm.

4. The fiber of claim 2, wherein the fiber further comprises collagen type I.

5. The fiber of claim 4, wherein the fiber is a nanofiber with an average diameter of about 200 nm.

6. The fiber of claim 1, wherein the polymer chains within a fiber are cross-linked or a fiber is further cross-linked to another fiber.

7. A scaffold comprising the fibers of claim 6.

8. A device comprising the fiber of claim 1 on its surface, wherein the device is selected from the group consisting of a glass slide, a culture plate, a plate well, a microwell, and a microfluidic chamber device.

9. An in vitro liver cell culture comprising the fiber of claim 6, and liver cells.

10. The in vitro liver cell culture of claim 9, wherein the liver cells are primary hepatocytes.

11. The in vitro liver cell culture of claim 9, further comprising supportive cells, optionally wherein the supportive cells are fibroblasts.

12. A liver model system for drug or chemical screening system, the system comprising the in vitro liver cell culture of claim 10.

13. A method of making a liver ECM fiber comprising:
a. obtaining or having obtained a decellularized porcine liver ECM;
b. dissolving the decellularized liver ECM in a solvent; and
c. electrospinning the solution to form a liver ECM fiber.

14. The method of claim 13, wherein the step b. further comprises dissolving collagen type I in the solvent to obtain a solution comprising a blend of decellularized liver ECM and collagen type I.

15. The method of claim 14, wherein the solvent is 1,1,1,3,3,3-hexafluoro-2-propanol (HFIP).

16. The method of claim 13, wherein the decellularized liver ECM is dissolved in the solvent at about 2 (w/v) % or about 5 (w/v) %.

17. The method of claim 13, wherein the electrospinning is conducted at about 15 to about 20 KV.

18. The method of claim 13, wherein the electrospinning is conducted on the surface of a device selected from the group consisting of a glass slide, a culture plate, a plate well, a microwell, and a microfluidic chamber device.

19. The method of claim 13, wherein step a. comprises:
i. obtaining a liver tissue comprising a whole liver, a liver lobe or a portion thereof;
ii. placing the liver tissue in deionized water at about 4° C. and replacing the deionized water every hour for about 12 hours;
iii. placing the liver tissue in a sodium dodecyl sulfate (SDS) solution at about 0.1% (w/v) at about 4° C., for about 8 hours to obtain decellularized liver ECM;
iv. removing the SDS solution, and placing the decellularized liver ECM in deionized water, and replacing the deionized water every hour for about 4 hours;
v. placing the decellularized liver ECM in 75% ethanol for about 8 hours at about 4° C.;
vi. lyophilizing the decellularized liver ECM;
vii. digesting lyophilized decellularized liver ECM by dissolving in 0.1 M HCl containing 100 mg of >2500 mg/units pepsin for about 8 hours;
viii. obtaining a supernatant from the HCl solution by centrifugation at 8000 g for 10 minutes;
ix. performing dialysis on the supernatant at 7KDa MWCO; and
x. lyophilizing the supernatant to obtain a decellularized liver ECM.

20. The method of claim 13, further comprising:
d. cross-linking the fiber using 1-ethyl-3-(3-dimethyl aminopropyl)carbodiimide, N-hydroxysuccinimide (NHS), or any combination thereof.

21. An in vitro liver cell culture comprising the fibers produced by the method of claim 13, and liver cells, wherein the liver cells are primary hepatocytes.

22. The in vitro liver cell culture of claim 21, further comprising supportive cells, optionally wherein the supportive cells are fibroblasts.
